SEQUENCE LISTING (3 sequences)

SEQ ID NO: 1 - DNA, 21 bp, Synthetic construct
  source 1..21: mol_type = other DNA; note = shRNA target sequences: shNT
caacaagatgaagagcaccaa

SEQ ID NO: 2 - DNA, 21 bp, Synthetic construct
  source 1..21: mol_type = other DNA; note = shRNA target sequences: shTCF4-13
gagactgaacggcaatctttc

SEQ ID NO: 3 - DNA, 21 bp, Synthetic construct
  source 1..21: mol_type = other DNA; note = shRNA target sequences: shTCF4-14
cacgaaatcttcggaggacaa

(19) United States
(12) Patent Application Publication
(10) Pub. No.: US 2025/0188545 A1
(43) Pub. Date: Jun. 12, 2025

(54) METHODS FOR PREDICTING RESPONSIVENESS OF LYMPHOMA TO DRUG AND METHODS FOR TREATING LYMPHOMA

(71) Applicant: Celgene Corporation, Summit, NJ (US)

(72) Inventors: Matthew STOKES, Scotch Plains, NJ (US); Anita GANDHI, Bernardsville, NJ (US); Chong Chris HUANG, Berwyn, PA (US)

(73) Assignee: Celgene Corporation, Summit, NJ (US)

(21) Appl. No.: 18/856,336

(22) PCT Filed: Apr. 14, 2023
(86) PCT No.: PCT/US2023/065794

§ 371 Date: Oct. 11, 2024

Related U.S. Application Data
(60) Provisional application No. 63/331,725, filed on Apr. 15, 2022.

(51) Int. Cl. C12Q 1/6886; A61K 31/475; A61K 31/573; A61K 31/675; A61K 31/704; A61K 39/395; A61P 35/00

(52) U.S. Cl. CPC C12Q 1/6886; A61K 31/475; A61K 31/573; A61K 31/675; A61K 31/704; A61K 39/3955; A61P 35/00; C12Q 2600/106; C12Q 2600/158

(57) ABSTRACT

Provided herein are methods of predicting the responsiveness of a lymphoma patient to a cancer treatment comprising clustering patients into subgroups of patients using gene expression levels. Also provided herein are methods of treating a lymphoma patient based on predicting the responsiveness of the lymphoma patient to a cancer treatment.

### CROSS-REFERENCE TO RELATED APPLICATIONS

This application claims priority to U.S. Provisional Application No. 63/331,725 filed Apr. 15, 2022, the content of which is incorporated by reference in its entirety herein.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “14247-722-228_SEQLISTING.xml”, was created on Mar. 29, 2023 and is 4,143 bytes in size.

### 1. FIELD

Provided herein are methods of predicting the responsiveness of a lymphoma patient to a cancer treatment. Also provided herein are methods of treating a lymphoma patient based on predicting the responsiveness of a lymphoma patient to a cancer treatment.

### 2. BACKGROUND

The non-Hodgkin lymphomas (NHLs) are a diverse group of blood cancers that include any kind of lymphoma except Hodgkin's lymphomas. Types of NHL vary significantly in their severity, from indolent to very aggressive. Less aggressive non-Hodgkin lymphomas are compatible with a long survival while more aggressive non-Hodgkin lymphomas can be rapidly fatal without treatment. They can be formed from either B-cells or T-cells. B-cell non-Hodgkin lymphomas include Burkitt lymphoma, chronic lymphocytic leukemia/small lymphocytic lymphoma (CLL/SLL), diffuse large B-cell lymphoma, follicular lymphoma, immunoblastic large cell lymphoma, precursor B-lymphoblastic lymphoma, and mantle cell lymphoma. T-cell non-Hodgkin lymphomas include mycosis fungoides, anaplastic large cell lymphoma, and precursor T-lymphoblastic lymphoma. Prognosis and treatment depend on the stage and type of disease.

Diffuse large B-cell lymphoma (DLBCL) accounts for approximately one-third of non-Hodgkin's lymphomas. While some DLBCL patients are cured with traditional chemotherapy, the rest die from the disease. Anticancer drugs cause rapid and persistent depletion of lymphocytes, possibly by direct apoptosis induction in mature T and B cells. See Stahnke et al., Blood, 2001, 98:3066-3073.

The diffuse large B-cell lymphomas (DLBCL) can be divided into distinct molecular subtypes according to their gene profiling patterns: germinal-center B-cell-like DLBCL (GCB-DLBCL), activated B-cell-like DLBCL (ABC-DLBCL), and primary mediastinal B-cell lymphoma (PMBL) or unclassified type. These subtypes are characterized by distinct differences in survival, chemo-responsiveness, and signaling pathway dependence, particularly the NF-κB pathway. See Kim et al., Journal of Clinical Oncology, 2007 ASCO Annual Meeting Proceedings Part I. vol 25, No. 18S (June 20 Supplement), 2007: 8082. See Bea et al., Blood, 2005; 106: 3183-3190; Ngo et al., Nature, 2011; 470:115-119. Such differences have prompted the search for more effective and subtype-specific treatment strategies in DLBCL.

Current cancer therapy, in general, can involve surgery, chemotherapy, hormonal therapy and/or radiation treatment to eradicate neoplastic cells in a patient (see, for example, Stockdale, 1998, Medicine, vol. 3, Rubenstein and Federman, eds., Chapter 12, Section IV). Recently, cancer therapy could also involve biological therapy or immunotherapy. All of these approaches pose significant drawbacks for the patient. Surgery, for example, may be contraindicated due to the health of a patient or may be unacceptable to the patient. Additionally, surgery may not completely remove neoplastic tissue. Radiation therapy is only effective when the neoplastic tissue exhibits a higher sensitivity to radiation than normal tissue. Radiation therapy can also often elicit serious side effects. Hormonal therapy is rarely given as a single agent. Although hormonal therapy can be effective, it is often used to prevent or delay recurrence of cancer after other treatments have removed the majority of cancer cells. Biological therapies and immunotherapies are limited in number and may produce side effects such as rashes or swellings, flu-like symptoms, including fever, chills and fatigue, digestive tract problems or allergic reactions.

In the context of DLBCL, treatment usually includes administration of a combination of chemotherapy and antibody therapy. The most widely used treatment of DLBCL is a combination of antibody rituximab (Rituxan) and chemotherapy drugs (cyclophosphamide, doxorubicin, vincristine, and prednisone (R-CHOP), and in some cases etoposide is added (R-EPOCH)). DLBCL also typically requires immediate treatment upon diagnosis due to how quickly the disease can advance. For some patients, DLBCL returns or becomes refractory following treatment. Several alternative treatments, some of which can include use of lenalidomide, are currently being tested in clinical trials for patients with newly diagnosed, relapsed or refractory DLBCL. See Czuczman et al., Clin Cancer Res., 2017, 23:4127-4137.

Among non-Hodgkin's lymphomas are also types of indolent lymphomas. Indolent B-cell lymphomas include, for example, follicular lymphoma, small lymphocytic lymphoma; nodal marginal zone B-cell lymphoma, lymphoplasmacytic lymphoma, anaplastic large cell lymphoma, primary cutaneous type, and mycosis fungoides. The clinical course of B-cell indolent lymphoma patients is characterized, among other features, by the risk of histologic transformation (HT) to an aggressive lymphoma, mostly DLBCL, and, less commonly, to Burkitt lymphoma (BL) or other types of aggressive lymphomas). See Montoto et al., Journal of Clinical Oncology, 2011, 29:1827-1834.

Due to the clinical and biological heterogeneity of lymphomas (particularly DLBCL), there is a significant need for effective methods of classifying subtypes of DLBCL for administering specific treatments. DLBCL has traditionally been classified by cell of origin (COO) subcategories based on tumor gene expression profiles and includes Activated B-Cell (ABC) and Germinal Center B-Cell (GCB) subtypes. See Alizadeh et al., Nature, 2000, 403:503-511; Wright et al., Proc. Natl. Acad. Sci. U.S.A, 2003, 100:9991-9996; Scott et al., Blood, 2014, 123:1214-1217. The GCB and ABC subtypes have different pathogenic mechanisms that may impact the outcomes of DLBCL patients on targeted therapies. See Nyman et al., Mod. Pathol., 2009, 22:1094-1101; Hans et al., Blood, 2004, 103:275-282; Choi et al., Clin. Cancer Res., 2009, 15:5494-5502; Meyer et al., J. Clin. Oncol., 2011, 29:200-207; Natkunam et al., J. Clin. Oncol., 2008, 26:447-454.

Recently, new classification models have focused on DNA alterations using tumor samples from patients treated with R-CHOP. See, e.g., Schmitz et al., N. Engl. J. Med., 2018, 378(15):1396-1407. However, a comprehensive integrative approach using transcriptomic data across both newly diagnosed (nd) and relapsed/refractory (r/r) DLBCL is yet to be accomplished. The present invention satisfies this and other needs.

### 3. SUMMARY OF THE INVENTION

In one aspect, provided herein is a method for predicting the responsiveness of a lymphoma patient to a cancer treatment comprising: (a) clustering reference lymphoma patients in a reference patient group into subgroups using the expression level of at least one gene in reference biological samples of the reference lymphoma patients; (b) determining a subgroup to which the lymphoma patient belongs based on the expression level of the at least one gene in a biological sample from the lymphoma patient; and (c) predicting the responsiveness of the lymphoma patient to a first cancer treatment based on the subgroup of the lymphoma patient.

In some embodiments, the method further comprises administering to the lymphoma patient a second cancer treatment.

In some embodiments, step (a) of the method further comprises generating clustering information defining relationships between the expression level of the at least one gene in the reference biological samples, and rearranging a heat map representation based on the clustering information.

In some embodiments, step (a) of the method uses a hierarchical method or a non-hierarchical method. In another aspect, step (a) uses iClusterPlus method.

In some embodiments, the reference lymphoma patients are clustered into 2-12 subgroups.

In some embodiments, the reference lymphoma patients are clustered into 7 subgroups.

In some embodiments, the method further comprises training a classifier model using the expression level of the at least one gene in the reference biological samples.

In some embodiments, the at least one gene is selected from the genes of Table 1.

In some embodiments, the at least one gene comprises five or more genes of Table 1.

In some embodiments, the at least one gene comprises all genes of Table 1.

In some embodiments, the classifier model is a grouped multinomial generalized linear model (GLM).

In some embodiments, the classifier model is a binary model.

In some embodiments, the method further comprises setting a threshold confidence level for at least one of the subgroups of step (a) to exclude patients that give lower confidence level clustering data from the at least one subgroup.

In some embodiments, the lymphoma is selected from the group consisting of diffuse large B-cell lymphoma (DLBCL), indolent B cell lymphoma, follicular lymphoma, small lymphocytic lymphoma, nodal marginal zone B-cell lymphoma, lymphoplasmacytic lymphoma, anaplastic large cell lymphoma, primary cutaneous type lymphoma, mycosis fungoides, chronic lymphocytic leukemia, and mantle cell Lymphoma.

In some embodiments, the lymphoma is DLBCL.

In some embodiments, the lymphoma is indolent B cell lymphoma, nodal marginal zone B-cell lymphoma, mantle cell lymphoma, or chronic lymphocytic leukemia.

In some embodiments, the reference patients in the reference patient group are clustered into subgroups A1-A7, and wherein: subgroup A1 comprises about 50% to about 60% patients having germinal center B-cell-like (GCB) DLBCL, about 30% to about 40% patients having activated B-cell like (ABC) DLBCL, about 10% to about 20% patients who are TME+ DLBCL patients, and about 30% to about 40% patients who are DHITsig+ DLBCL patients; (ii) subgroup A2 comprises about 80% to about 90% patients having GCB DLBCL, about 0% to about 5% patients having ABC DLBCL, about 15% to about 25% patients who are TME+ DLBCL patients, and about 25% to about 35% patients who are DHITsig+ DLBCL patients; (iii) subgroup A3 comprises about 40% to about 55% patients having GCB DLBCL, about 30% to about 45% patients having ABC DLBCL, about 40% to about 50% patients who are TME+ DLBCL patients, and about 20% to about 30% patients who are DHITsig+ DLBCL patients; (iv) subgroup A4 comprises about 25% to about 35% patients having GCB DLBCL, about 40% to about 50% patients having ABC DLBCL, about 30% to about 40% patients who are TME+ DLBCL patients, and about 10% to about 20% patients who are DHITsig+ DLBCL patients; (v) subgroup A5 comprises about 20% to about 40% patients having GCB DLBCL, about 45% to about 65% patients having ABC DLBCL, about 30% to about 40% patients who are TME+ DLBCL patients, and about 0% to about 10% patients who are DHITsig+ DLBCL patients; (vi) subgroup A6 comprises about 30% to about 40% patients having GCB DLBCL, about 40% to about 50% patients having ABC DLBCL, about 75% to about 95% patients who are TME+ DLBCL patients, and about 0% to about 10% patients who are DHITsig+ DLBCL patients; and (vii) subgroup A7 comprises about 0% to about 10% patients having GCB DLBCL, about 80% to about 90% patients having ABC DLBCL, about 0% to about 10% patients who are TME+ DLBCL patients, and about 0% to about 15% patients who are DHITsig+ DLBCL patients.

In some embodiments, the first cancer treatment is a combination treatment with rituximab, cyclophosphamide, doxorubicin, vincristine, and prednisone (R-CHOP).

In some embodiments, when the lymphoma patient is determined to belong to subgroup A1, the method comprises predicting that the patient is not likely to be responsive to the first cancer treatment.

In some embodiments, when the lymphoma patient is determined to belong to subgroup A2, the method comprises predicting that the patient is not likely to be responsive to the first cancer treatment.

In some embodiments, when the lymphoma patient is determined to belong to subgroup A3, the method comprises predicting that the patient is not likely to be responsive to the first cancer treatment.

In some embodiments, when the lymphoma patient is determined to belong to subgroup A4, the method comprises predicting that the patient is not likely to be responsive to the first cancer treatment.

In some embodiments, when the lymphoma patient is determined to belong to subgroup A5, the method comprises predicting that the patient is not likely to be responsive to the first cancer treatment.

In some embodiments, when the lymphoma patient is determined to belong to subgroup A6, the method comprises predicting that the patient is not likely to be responsive to the first cancer treatment.

In some embodiments, when the lymphoma patient is determined to belong to subgroup A7, the method comprises predicting that the patient is not likely to be responsive to the first cancer treatment.

In some embodiments, the second cancer treatment is R-CHOP.

In some embodiments, the second cancer treatment is not R-CHOP.

In some embodiments the second cancer treatment is a bromodomain and extra-terminal (BET) inhibitor, or a cyclin dependent kinase (CDK) inhibitor.

In one aspect, provided herein is a method for predicting the responsiveness of a lymphoma patient to a cancer treatment comprising: (a) determining the expression level of at least one gene of Table 1 in a biological sample from a lymphoma patient; (b) comparing the expression level of the at least one gene of step (a) with the expression level of the at least one gene in a reference biological sample from a reference lymphoma patient, wherein the reference lymphoma patient is responsive to the cancer treatment, and wherein if the expression level of the at least one gene in the biological sample is similar to the expression level of the at least one gene in the reference biological sample, it indicates that the lymphoma patient is not likely to be responsive to the cancer treatment.

In some embodiments, the at least one gene comprising five or more genes of Table 1.

In one aspect, provided herein is a method for predicting the responsiveness of a lymphoma patient to a cancer treatment comprising: (a) determining the expression level of at least one gene of Table 1 in a biological sample of a lymphoma patient; and (b) comparing the expression level of the at least one gene in the biological sample to: (i) the expression level of the at least one gene in biological samples from lymphoma patients who are responsive to the cancer treatment, and (ii) the expression level of the at least one gene in biological samples from lymphoma patients who are not responsive to the cancer treatment, wherein if the expression level of (a) is similar to the expression level of (i), it indicates that the first lymphoma patient is likely to be responsive to the cancer treatment; and if the expression level of (a) is similar to the expression level of (ii), it indicates that the first lymphoma patient is not likely to be responsive to the cancer treatment.

In one aspect, provided herein is a method of treating a lymphoma patient comprising: (i) identifying a lymphoma patient who is likely to be responsive to the cancer treatment; and (ii) administering to the lymphoma patient the cancer treatment.

In one aspect, provided herein is a method of treating a lymphoma patient comprising: (i) identifying a lymphoma patient who is not likely to be responsive to the cancer treatment; and (ii) administering to the lymphoma patient an alternative cancer treatment.

In some embodiments, the cancer treatment is R-CHOP.

In some embodiments, the alternative cancer treatment is a BET inhibitor, or a CDK inhibitor.

In some embodiments, the lymphoma is selected from the group consisting of DLBCL, indolent B cell lymphoma, follicular lymphoma, small lymphocytic lymphoma, nodal marginal zone B-cell lymphoma, lymphoplasmacytic lymphoma, anaplastic large cell lymphoma, primary cutaneous type lymphoma, mycosis fungoides, chronic lymphocytic leukemia, and mantle cell Lymphoma.

In some embodiments, the lymphoma is DLBCL.

In some embodiments, the lymphoma is DLBCL, indolent B cell lymphoma, follicular lymphoma, nodal marginal zone B-cell lymphoma, mantle cell lymphoma, or chronic lymphocytic leukemia.

In some embodiments, the expression levels of all genes of Table 1 are determined in (a) and compared in (b) as described herein.

In some embodiments, the biological samples are tumor biopsy samples.

In some embodiments, determining the expression level of the at least one gene comprises detecting the presence or amount of at least one complex in the biological sample, wherein the presence or amount of the at least one complex indicates the expression level of the at least one gene.

In some embodiments, the at least one complex is a hybridization complex.

In some embodiments, the at least one complex is detectably labeled.

In some embodiments, determining the expression level of the at least one gene comprises detecting the presence or the amount of at least one reaction product in the biological sample, wherein the presence or amount of the at least one reaction product indicates the expression level of the at least one gene.

In some embodiments, the at least one reaction product is detectably labeled.

In some embodiments, the reference lymphoma patient is a refractory DLBCL patient, a relapsed DLBCL patient, or a newly diagnosed DLBCL patient.

In some embodiments, the lymphoma patient is a refractory DLBCL patient, a relapsed DLBCL patient, or a newly diagnosed DLBCL patient.

In some embodiments, the lymphoma patient is a GCB DLBCL patient or an ABC DLBCL patient.

In some embodiments, the lymphoma patient is a DHITsig+ DLBCL patient or a DHITsig− DLBCL patient.

### 4. BRIEF DESCRIPTION OF THE FIGURES

FIGS. 1A-1E depict that unsupervised clustering was applied to a large cohort of patient-derived RNAseq data to identify biologically homogeneous segments of DLBCL. FIG. 1A depicts a schematic of data transformation, unsupervised clustering, and classifier training methodology. FIG. 1B depicts a co-clustering frequency heatmap that identified sample clusters that consistently group together over repeated subsampling runs. FIG. 1C depicts top 50 up- and down-regulated genes per cluster. FIG. 1D depicts top 50 up- and down-regulated genes per cluster for independent cohort MER. FIG. 1E depicts top 50 up- and down-regulated genes per cluster for independent cohort REMoDL-B.

FIGS. 2A-2I depict clinical outcome stratified by cluster. FIG. 2A depicts event-free survival (EFS) of RCHOP-treated ABC-COO patients in the ROBUST subset of the Discovery cohort. FIG. 2B depicts EFS of RCHOP-treated patients in the MER replication cohort. FIG. 2C depicts progression free survival (PFS) of RCHOP-treated patients in the REMoDL-B replication cohort. FIGS. 2D-2F depict EFS/PFS of ROBUST (FIG. 2D), MER (FIG. 2E), and REMoDL-B (FIG. 2F) cohorts classified as A7/non-A7. FIGS. 2G-2I depict forest plot of the log-odds ratio of belonging to A7 given the presence of various clinical factors in ROBUST (FIG. 2G), MER (FIG. 211), and REMoDL-B (FIG. 2I) cohorts.

FIGS. 3A-3E depict biological features of the discovered subtypes. FIG. 3A depicts scatter plot of the Discovery cohort in the space of Reddy COO score vs. TME26 score. FIG. 3B depicts a collection of curated DLBCL signatures showing cluster-discriminative signals. FIG. 3C depicts cluster-associated single nucleotide variants (SNVs) (ROBUST). FIG. 3D depicts cluster-associated copy number variants (CNVs) (ROBUST). FIG. 3E depicts representative immunohistochemistry (IHC) images of A6 and A7.

FIGS. 4A-4G depict biological characteristics of A7. FIG. 4A depicts significantly dysregulated hallmark pathways in A7 (Discovery), ranked by p-value with Normalized Enrichment Scores (NES) shown. FIG. 4B depicts MYC gene expression by A7 status across cohorts. FIG. 4C depicts representative MYC staining in A7. FIG. 4D depicts copy number amplification/deletion frequency in A7. FIG. 4E depicts a western blot that showed expression of TCF4 in ABC-like DLBCL cell lines. FIG. 4F depicts a western blot that showed expression of MYC and TCF4 in TCF4 knockdown ABC-like DLBCL cell lines. GAPDH was used as a loading control in the western blots. FIG. 4G depicts a cell proliferation assay of control (shNT) and TCF4 knockdown (shTCF4) in ABC-like DLBCL cell lines. Error bars represent the SEM of technical triplicates (SEM<1 not shown).

FIGS. 5A-5B depict clinical utility of A7. A7 was predictive of outcome in RCHOP-treated patients in both ROBUST (FIG. 5A) and REMoDL-B (FIG. 5B), but less predictive of outcome in R2CHOP (lenalidomide in combination with R-CHOP)- or RBCHOP (bortezomib in combination with R-CHOP)-treated patients.

FIGS. 6A-6D depict that samples clustering into A8 had significantly lower alignment to (FIG. 6A) coding regions and significantly higher rates of (FIG. 6B) intergenic, (FIG. 6C) ribosomal, and (FIG. 6D) unaligned reads than other samples (Discovery).

FIG. 7 depicts confusion matrix of classifier output in the training dataset (Discovery).

FIGS. 8A-8C depict Robust (FIG. 8A), Mer (FIG. 8B), REMoDL-B (FIG. 8C) survival probabilities. A7 stratified risk within clinically defined international prognostic index (IPI) groups.

FIGS. 9A-9B depict significantly dysregulated pathways in each of the discovered clusters when comparing each cluster to all others (Discovery) (FIG. 9A). FIG. 9B depicts pathway enrichment scores that were generally concordant between the Discovery and MER datasets, with most pathways sharing directionality and significance (colored in red). Cluster A4 was the exception to this trend, with many pathways showing reversed directionality between Discovery and MER.

FIGS. 10A-10F depict copy number aberration prevalence in each cluster compared to the remaining population (ROBUST+MER). FIG. 10A depicts A1 DLBCL vs. Non-A1 (combined ROBUST+MER), FIG. 10B depicts A2 DLBCL vs. Non-A2 (combined ROBUST +MER), FIG. 10C depicts A3 DLBCL vs. Non-A3 (combined ROBUST+MER), FIG. 10D depicts A4 DLBCL vs. Non-A4 (combined ROBUST+MER), FIG. 10E depicts A5 DLBCL vs. Non-A5 (combined ROBUST+MER), and FIG. 10F depicts A6 DLBCL vs. Non-A6 (combined ROBUST+MER).

FIGS. 11A-11F depict major immune types detected by Multiplexed Ion Beam Imaging (MIBI) in part of the ROBUST plus cohort (n=43). Cellular abundance was expressed as percentage to total nucleated cells within a field of view (FOV). FIG. 11A depicts CD3 total T cells; FIG. 11B depicts CD4 T cells; FIG. 11C depicts CD8 T-cells; FIG. 11D depicts CD163 macrophage/monocytes; FIG. 11E depicts CD68 macrophages; and FIG. 11F depicts CD11c dendritic cells.

FIGS. 12A-12E depict genomic characteristics of A7. FIG. 12A depicts A7-associated genomic events and their associated variant allele frequency (VAF) and cancer cell fraction (CCF). A7-associated CNAs tended to be highly clonal, with a CCF of 100% in most samples. Most A7 mutation events, on the other hand, were observed at least partially or even exclusively among subclones. FIG. 12B depicts A7-associated CNA (MER). FIG. 12C depicts MYC expression by A7 status (MER). FIG. 12D depicts tumor purity by A7 status (ROBUST). FIG. 12E depicts tumor purity that had near-zero correlation with MYC expression (ROBUST).

FIGS. 13A-13B depict TCF4 mRNA expression in patients with indicated TCF4 copy number alternation (ROBUST) (FIG. 13A) or in DLBCL cell lines (FIG. 13B).

FIGS. 14A-14B depict a comparison of A7 and MCD in the Novel clusters compared to LymphGen (NCI cohort). FIG. 14A depicts PFS of immunochemotherapy-treated patients stratified by MCD and A7 status. FIG. 14B depicts Sankey plot illustrating co-occurrence of LymphGen clusters and A1-A7.

FIGS. 15A-15D depict sankey plot and associated confusion matrices of the discovered subtypes compared to the LymphGen classifier output in ROBUST (FIGS. 15A and 15C) and MER (FIGS. 15B and 15D). The MCD subtype (based on the co-occurrence of MYD88^{L265P} and CD79B mutations as described in Schmitz et al., 2018, New England Journal of Medicine, 378(15), 1396-1407) was enriched for A7 patients, while the EZB subtype (based on EZH2 mutations and BCL2 translocations as described in Schmitz et al., 2018, New England Journal of Medicine, 378(15), 1396-1407) was enriched for GCB-like clusters A2 and A3. Although a statistically significant association existed between the classification methods (Fisher p=0.0005 in ROBUST, p=0.001 in MER), there was a great deal of heterogeneity and no clear one-to-one mapping between any of the subtypes.

FIG. 16 depicts novel clusters compared to LymphGen (NCI cohort). PCA plot of the Discovery, MER, and REMoDL-B datasets after normalization showed no dataset-specific differences.

FIGS. 17A-17C depict mutation landscape (Chapuy genes), which was sorted by: mutation count (FIG. 17A), by significance (corrected for gene length) (FIG. 17B), and Chapuy figure (for reference) (FIG. 17C).

FIGS. 18A-18D depict expression of proteins encoded by genes of chromosome 18. FIG. 18A depicts expression by copy number amplification on Chr18. FIG. 18B depicts a western blot that showed expression of MTAP in DLBCL cell lines. FIG. 18C depicts a western blot that showed expression of SDMA and PRMT5 in DLBCL cell lines. GAPDH, loading control. FIG. 18D depicts a cell proliferation assay of control (shNT) and PRMT5 knockdown (sh PRMT5) in ABC-like DLBCL cell lines. Error bars represent the SEM of technical triplicates.

### 5. DETAILED DESCRIPTION OF THE INVENTION

### 5.1 Definitions

As used herein, the term “lymphoma” includes, but is not limited to, Hodgkin's lymphoma, non-Hodgkin's lymphoma, diffuse large B-Cell lymphoma, indolent B-cell lymphoma, follicular lymphoma, small lymphocytic lymphoma, nodal marginal zone B-cell lymphoma, lymphoplasmacytic lymphoma, anaplastic large cell lymphoma, primary cutaneous type lymphoma, cutaneous T-Cell lymphoma, cutaneous B-Cell lymphoma, mycosis fungoide, mantle cell lymphoma, and chronic lymphocytic leukemia.

As used herein, and unless otherwise specified, the terms “treat,” “treating,” and “treatment” refer to an action that occurs while a patient is suffering from the specified cancer (a specific type of lymphoma, e.g., DLBCL), which reduces the severity of the cancer or retards or slows the progression of the cancer.

The term “sensitivity” or “sensitive” when made in reference to a cancer treatment is a relative term which refers to the degree of effectiveness of the cancer treatment in lessening or decreasing the progress of a tumor or the cancer being treated. For example, the term “increased sensitivity” when used in reference to treatment of a cell or tumor in connection with a compound refers to an increase of, at least about 5%, or more, in the effectiveness of the cancer treatment.

As used herein, and unless otherwise specified, the term “therapeutically effective amount” of a cancer treatment is an amount sufficient to provide a therapeutic benefit in the treatment or management of a cancer, or to delay or minimize one or more symptoms associated with the presence of the cancer. A therapeutically effective amount of a compound means an amount of therapeutic agent, alone or in combination with other therapies, which provides a therapeutic benefit in the treatment or management of the cancer. The term “therapeutically effective amount” can encompass an amount that improves overall therapy, reduces or avoids symptoms or causes of cancer, or enhances the therapeutic efficacy of another therapeutic agent. The term also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician.

The term “responsiveness” or “responsive” when used in reference to a cancer treatment refers to the degree of effectiveness of the treatment in lessening or decreasing the symptoms of a cancer, e.g., DLBCL, being treated. For example, the term “increased responsiveness” when used in reference to a treatment of a cell or a subject refers to an increase in the effectiveness in lessening or decreasing the symptoms of the disease compared to a reference treatment (e.g., of the same cell or subject, or of a different cell or subject) when measured using any methods known in the art. In certain embodiments, the increase in the effectiveness is at least about 5%, at least about 10%, at least about 20%, at least about 30%, at least about 40%, or at least about 50%.

As used herein, the terms “effective subject response,” “effective patient response,” and “effective patient tumor response” refer to any increase in the therapeutic benefit to the patient. An “effective patient tumor response” can be, for example, about 5%, about 10%, about 25%, about 50%, or about 100% decrease in the rate of progress of the tumor. An “effective patient tumor response” can be, for example, about 5%, about 10%, about 25%, about 50%, or about 100% decrease in the physical symptoms of a cancer. An “effective patient tumor response” can also be, for example, about 5%, about 10%, about 25%, about 50%, about 100%, about 200%, or more increase in the response of the patient, as measured by any suitable means, such as gene expression, cell counts, assay results, tumor size, etc.

An improvement in the cancer (e.g., DLBCL or a subtype thereof) or cancer-related disease can be characterized as a complete or partial response. “Complete response” refers to an absence of clinically detectable disease with normalization of any previously abnormal radiographic studies, bone marrow, and cerebrospinal fluid (CSF) or abnormal monoclonal protein measurements. “Partial response” refers to at least about 10%, about 20%, about 30%, about 40%, about 50%, about 60%, about 70%, about 80%, or about 90% decrease in all measurable tumor burden (i.e., the number of malignant cells present in the subject, or the measured bulk of tumor masses or the quantity of abnormal monoclonal protein) in the absence of new lesions. The term “treatment” contemplates both a complete and a partial response.

The term “likelihood” generally refers to an increase in the probability of an event. The term “likelihood” when used in reference to the effectiveness of a patient tumor response generally contemplates an increased probability that the rate of tumor progress or tumor cell growth will decrease. The term “likelihood” when used in reference to the effectiveness of a patient tumor response can also generally mean the increase of indicators, such as mRNA or protein expression, that may evidence an increase in the progress in treating the tumor.

The term “predict” generally means to determine or tell in advance. When used to “predict” the effectiveness of a cancer treatment, for example, the term “predict” can mean that the likelihood of the outcome of the cancer treatment can be determined at the outset, before the treatment has begun, or before the treatment period has progressed substantially.

As used herein, the term “source” when used in reference to a reference sample refers to the origin of a sample. For example, a sample that is taken from blood would have a reference sample that is also taken from blood. Similarly, a sample that is taken from bone marrow would have a reference sample that is also taken from the bone marrow.

As used herein, the term “refractory” or “resistant” refers to a disorder, disease, or condition that has not responded to prior treatment that can include one or more lines of therapy. In some embodiments, the disorder, disease, or condition has been previously treated one, two, three or four lines of therapy. In some embodiments, the disorder, disease, or condition has been previously treated with two or more lines of treatment, and has less than a complete response (CR) to most recent systemic therapy containing regimen.

As used herein, the term “relapsed” refers to a disorder, disease, or condition that responded to treatment (e.g., achieved a complete response) then had progression. The treatment can include one or more lines of therapy.

The term “expressed” or “expression” as used herein refers to the transcription from a gene to give an RNA nucleic acid molecule at least complementary in part to a region of one of the two nucleic acid strands of the gene. The term “expressed” or “expression” as used herein also refers to the translation from the RNA molecule to give a protein, a polypeptide, or a portion thereof. A “biological marker” or “biomarker” is a substance whose detection indicates a particular biological state, such as, for example, the presence of a type of cancer. In some embodiments, biomarkers can be determined individually. In other embodiments, several biomarkers can be measured simultaneously.

The terms “polypeptide” and “protein,” as used interchangeably herein, refer to a polymer of three or more amino acids in a serial array, linked through peptide bonds. The term “polypeptide” includes proteins, protein fragments, protein analogues, oligopeptides, and the like. The term “polypeptide” as used herein can also refer to a peptide. The amino acids making up the polypeptide may be naturally derived, or may be synthetic. The polypeptide can be purified from a biological sample. The polypeptide, protein, or peptide also encompasses modified polypeptides, proteins, and peptides, e.g., glycopolypeptides, glycoproteins, or glycopeptides; or lipopolypeptides, lipoproteins, or lipopeptides.

The term “antibody,” “immunoglobulin,” or “Ig” as used interchangeably herein, encompasses fully assembled antibodies and antibody fragments that retain the ability to specifically bind to the antigen. Antibodies provided herein include, but are not limited to, synthetic antibodies, monoclonal antibodies, polyclonal antibodies, recombinantly produced antibodies, multispecific antibodies (including bi-specific antibodies), human antibodies, humanized antibodies, chimeric antibodies, intrabodies, single-chain Fvs (scFv) (e.g., including monospecific, bispecific, etc.), camelized antibodies, Fab fragments, F(ab′) fragments, disulfide-linked Fvs (sdFv), anti-idiotypic (anti-Id) antibodies, and epitope-binding fragments of any of the above. In particular, antibodies provided herein include immunoglobulin molecules and immunologically active portions of immunoglobulin molecules, i.e., antigen binding domains or molecules that contain an antigen-binding site that immunospecifically binds to CRBN antigen (e.g., one or more complementarity determining regions (CDRs) of an anti-CRBN antibody). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. In some embodiments, the anti-CRBN antibodies are fully human, such as fully human monoclonal CRBN antibodies. In certain embodiments, antibodies provided herein are IgG antibodies, or a subclass thereof (e.g., human IgG1 or IgG4).

The terms “antigen binding domain,” “antigen binding region,” “antigen binding fragment,” and similar terms refer to the portion of an antibody that comprises the amino acid residues that interact with an antigen and confer on the binding agent its specificity and affinity for the antigen (e.g., the CDR). The antigen binding region can be derived from any animal species, such as rodents (e.g., rabbit, rat, or hamster) and humans. In some embodiments, the antigen-binding region is of human origin.

The term “epitope” as used herein refers to a localized region on the surface of an antigen that is capable of binding to one or more antigen binding regions of an antibody, that has antigenic or immunogenic activity in an animal, such as a mammal (e.g., a human), and that is capable of eliciting an immune response. An epitope having immunogenic activity is a portion of a polypeptide that elicits an antibody response in an animal. An epitope having antigenic activity is a portion of a polypeptide to which an antibody immunospecifically binds as determined by any method well known in the art, for example, by the immunoassays described herein. Antigenic epitopes need not necessarily be immunogenic. Epitopes usually consist of chemically active surface groupings of molecules, such as amino acids or sugar side chains, and have specific three-dimensional structural characteristics as well as specific charge characteristics. A region of a polypeptide contributing to an epitope may be contiguous amino acids of the polypeptide, or the epitope may come together from two or more non-contiguous regions of the polypeptide. The epitope may or may not be a three-dimensional surface feature of the antigen.

The terms “fully human antibody” and “human antibody” are used interchangeably herein and refer to an antibody that comprises a human variable region and, in some embodiments, a human constant region. In specific embodiments, the terms refer to an antibody that comprises a variable region and a constant region of human origin. The term “fully human antibody” includes antibodies having variable and constant regions corresponding to human germline immunoglobulin sequences as described by Kabat et al., Sequences of Proteins of Immunological Interest, U.S. Department of Health and Human Services, NIH Publication No. 91-3242 (5th ed. 1991).

The phrase “recombinant human antibody” includes human antibodies that are prepared, expressed, created, or isolated by recombinant means, such as antibodies expressed using a recombinant expression vector transfected into a host cell, antibodies isolated from a recombinant, combinatorial human antibody library, antibodies isolated from an animal (e.g., a mouse or a cow) that is transgenic and/or transchromosomal for human immunoglobulin genes (see, e.g., Taylor et al., Nucl. Acids Res., 1992, 20:6287-6295) or antibodies prepared, expressed, created, or isolated by any other means that involves splicing of human immunoglobulin gene sequences to other DNA sequences. Such recombinant human antibodies can have variable and constant regions derived from human germline immunoglobulin sequences. See Kabat et al., Sequences of Proteins of Immunological Interest, U.S. Department of Health and Human Services, NIH Publication No. 91-3242 (5th ed. 1991). In certain embodiments, however, such recombinant human antibodies are subjected to in vitro mutagenesis (or, when an animal transgenic for human Ig sequences is used, in vivo somatic mutagenesis) and thus the amino acid sequences of the heavy chain variable and light chain variable regions of the recombinant antibodies are sequences that, while derived from and related to human germline heavy chain variable and light chain variable sequences, may not naturally exist within the human antibody germline repertoire in vivo.

The term “monoclonal antibody” refers to an antibody obtained from a population of homogenous or substantially homogeneous antibodies, and each monoclonal antibody will typically recognize a single epitope on the antigen. In some embodiments, a “monoclonal antibody,” as used herein, is an antibody produced by a single hybridoma or other cell, wherein the antibody immunospecifically binds to only an epitope as determined, e.g., by ELISA or other antigen-binding or competitive binding assay known in the art or in the Examples provided herein. The term “monoclonal” is not limited to any particular method for making the antibody. For example, monoclonal antibodies provided herein may be made by the hybridoma method as described in Kohler et al., Nature, 1975, 256:495-497, or may be isolated from phage libraries using the techniques as described herein. Other methods for the preparation of clonal cell lines and of monoclonal antibodies expressed thereby are well known in the art. See, e.g., Short Protocols in Molecular Biology, Chapter 11 (Ausubel et al., eds., John Wiley and Sons, New York, 5th ed. 2002). Other exemplary methods of producing other monoclonal antibodies are provided in the Examples herein.

“Polyclonal antibodies” as used herein refers to an antibody population generated in an immunogenic response to a protein having many epitopes and thus includes a variety of different antibodies directed to the same or to different epitopes within the protein. Methods for producing polyclonal antibodies are known in the art. See, e.g., Short Protocols in Molecular Biology, Chapter 11 (Ausubel et al., eds., John Wiley and Sons, New York, 5th ed. 2002).

The term “level” refers to the amount, accumulation, or rate of a molecule. A level can be represented, for example, by the amount or the rate of synthesis of a messenger RNA (mRNA) encoded by a gene, the amount or the rate of synthesis of a polypeptide or protein encoded by a gene, or the amount or the rate of synthesis of a biological molecule accumulated in a cell or biological fluid. The term “level” refers to an absolute amount of a molecule in a sample or a relative amount of the molecule, determined under steady-state or non-steady-state conditions.

An mRNA that is “upregulated” is generally increased upon a given treatment or condition. An mRNA that is “downregulated” generally refers to a decrease in the level of expression of the mRNA in response to a given treatment or condition. In some situations, the mRNA level can remain unchanged upon a given treatment or condition. An mRNA from a patient sample can be “upregulated” when received a treatment, as compared to a non-treated control. This upregulation can be, for example, an increase of about 5%, about 10%, about 20%, about 30%, about 40%, about 50%, about 60%, about 70%, about 80%, about 90%, about 100%, about 200%, about 300%, about 500%, about 1,000%, about 5,000%, or more of the comparative control mRNA level. Alternatively, an mRNA can be “downregulated”, or expressed at a lower level, in response to administration of certain compounds or other agents. A downregulated mRNA can be, for example, present at a level of about 99%, about 95%, about 90%, about 80%, about 70%, about 60%, about 50%, about 40%, about 30%, about 20%, about 10%, about 1%, or less of the comparative control mRNA level.

Similarly, the level of a polypeptide or protein biomarker from a patient sample can be increased when received a treatment, as compared to a non-treated control. This increase can be about 5%, about 10%, about 20%, about 30%, about 40%, about 50%, about 60%, about 70%, about 80%, about 90%, about 100%, about 200%, about 300%, about 500%, about 1,000%, about 5,000%, or more of the comparative control protein level. Alternatively, the level of a protein biomarker can be decreased in response to administration of certain compounds or other agents. This decrease can be, for example, present at a level of about 99%, about 95%, about 90%, about 80%, about 70%, about 60%, about 50%, about 40%, about 30%, about 20%, about 10%, about 1%, or less of the comparative control protein level.

The terms “determining,” “measuring,” “evaluating,” “assessing,” and “assaying” as used herein generally refer to any form of measurement, and include determining whether an element is present or not. These terms include quantitative and/or qualitative determinations. Assessing may be relative or absolute. “Assessing the presence of” can include determining the amount of something present, as well as determining whether it is present or absent.

The terms “isolated” and “purified” refer to isolation of a substance (such as mRNA, DNA, or protein) such that the substance comprises a substantial portion of the sample in which it resides, i.e., greater than the portion of the substance that is typically found in its natural or un-isolated state. Typically, a substantial portion of the sample comprises, e.g., greater than 1%, greater than 2%, greater than 5%, greater than 10%, greater than 20%, greater than 50%, or more, usually up to about 90%-100% of the sample. For example, a sample of isolated mRNA can typically comprise at least about 1% total mRNA. Techniques for purifying polynucleotides are well known in the art and include, for example, gel electrophoresis, ion-exchange chromatography, affinity chromatography, flow sorting, and sedimentation according to density.

As used herein, the term “bound” indicates direct or indirect attachment. In the context of chemical structures, “bound” (or “bonded”) may refer to the existence of a chemical bond directly joining two moieties or indirectly joining two moieties (e.g., via a linking group or any other intervening portion of the molecule). The chemical bond may be a covalent bond, an ionic bond, a coordination complex, hydrogen bonding, van der Waals interactions, or hydrophobic stacking, or may exhibit characteristics of multiple types of chemical bonds. In certain instances, “bound” includes embodiments where the attachment is direct and embodiments where the attachment is indirect.

The term “sample” as used herein relates to a material or mixture of materials, typically, although not necessarily, in fluid form, comprising one or more components of interest. In some embodiments, a sample can be a biological sample. “Biological sample” as used herein refers to a sample obtained from a biological subject, including a sample of biological tissue or fluid origin, obtained, reached, or collected in vivo or in situ. A biological sample also includes samples from a region of a biological subject comprising precancerous or cancer cells or tissues. Such samples can be, but are not limited to, organs, tissues, and cells isolated from a mammal. Exemplary biological samples include but are not limited to cell lysate, a cell culture, a cell line, a tissue, oral tissue, gastrointestinal tissue, an organ, an organelle, a biological fluid, a blood sample, a urine sample, a skin sample, and the like. Preferred biological samples include, but are not limited to, whole blood, partially purified blood, PBMC, tissue biopsies, and the like.

The term “analyte” as used herein refers to a known or unknown component of a sample.

The term “capture agent” as used herein refers to an agent that binds an mRNA or protein through an interaction that is sufficient to permit the agent to bind and to concentrate the mRNA or protein from a heterogeneous mixture.

As used herein and unless otherwise indicated, the term “pharmaceutically acceptable salt” encompasses non-toxic acid and base addition salts of the compound to which the term refers. Acceptable non-toxic acid addition salts include those derived from organic and inorganic acids know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like. Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts comprising pharmacologically acceptable cations such as, but not limited to, alkali metal or alkaline earth metal salts (calcium, magnesium, sodium, or potassium salts in particular). Suitable organic bases include, but are not limited to, N,N-dibenzylethylenediamine, chloroprocaine, choline, diethanolamine, ethylenediamine, meglumaine (N-methylglucamine), lysine, and procaine.

As described herein, the term “second active agent” refers to any additional treatment that is biologically active. It is understood that the second active agent can be a hematopoietic growth factor, cytokine, anti-cancer agent, antibiotic, cox-2 inhibitor, immunomodulatory agent, immunosuppressive agent, corticosteroid, therapeutic antibody that specifically binds to a cancer antigen or a pharmacologically active mutant, or derivative thereof. Exemplary second active agents include, but are not limited to, an HDAC inhibitor (e.g., panobinostat, romidepsin, or vorinostat), a BCL2 inhibitor (e.g., venetoclax), a BTK inhibitor (e.g., ibrutinib or acalabrutinib), an mTOR inhibitor (e.g., everolimus), a PI3K inhibitor (e.g., idelalisib), a PKCβ inhibitor (e.g., enzastaurin), a SYK inhibitor (e.g., fostamatinib), a JAK2 inhibitor (e.g., fedratinib, pacritinib, ruxolitinib, baricitinib, gandotinib, lestaurtinib, or momelotinib), an Aurora A kinase inhibitor (e.g., alisertib), an EZH2 inhibitor (e.g., tazemetostat, GSK126, CPI-1205, 3-deazaneplanocin A, EPZ005687, El1, UNC1999, or sinefungin), a BET inhibitor (e.g., birabresib or 4 [2-(cyclopropylmethoxy)-5-(methanesulfonyl)phenyl]-2-methylisoquinolin-1(2H)-one), a hypomethylating agent (e.g., 5-azacytidine or decitabine), a chemotherapy (e.g., bendamustine, doxorubicin, etoposide, methotrexate, cytarabine, vincristine, ifosfamide, or melphalan), or an epigenetic compound (e.g., a DOT1L inhibitor such as pinometostat, a HAT inhibitor such as C646, a WDR5 inhibitor such as OICR-9429, a HDAC6 inhibitor such as ACY-241, a DNMT1 selective inhibitor such as GSK3484862, a LSD-1 inhibitor such as Compound C or seclidemstat, a G9A inhibitor such as UNC 0631, a PRMT5 inhibitor such as GSK3326595, a BRPF1B/2 inhibitor such as OF-1, a BRD9/7 inhibitor such as LP99, a SUV420H1/H2 inhibitor such as A-196, a Menin-MLL inhibitor such as MI-503, a CARM1 inhibitor such as EZM2302, a BRD9 such as an inhibitor dBrd9), aiolos/ikaros degrading cereblon E3 ligase modulator (CELMoDs), CREBBp2 inhibitors, anti-CD79b antibody, CD19 CAR-T, inhibitors of p53 (nutlins), Bcl6 inhibitors, CREBBp2 CELMoDs, CD79b CELMoDs, CD19 CELMoDs, p53 (nutlins) CELMoDs, Bcl6 CELMoDs, inhibitors of ligand directed degradation (LDD) of CREBBP2, inhibitors of LDD of CD79b, inhibitors of LDD of CD19, inhibitors of LDD of p53 (nutlins), inhibitors of LDD of Bcl6, inhibitors of LDD of CK1a, inhibitors of LDD of IRAK4 (e.g., in MYD88 L265p lymphoma), MALT1 inhibitors such as JNJ-67856633, MAT2A inhibitors (e.g., for 9p21 deletions), anti-CD3×anti-CD19 bispecific antibodies, and anti-CD3×anti-CD20 bispecific antibodies.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The use of the word “a” or “an” when used in conjunction with the term “comprising” in the claims and/or the specification can mean “one,” but it is also consistent with the meaning of “one or more,” “at least one,” and “one or more than one.”

As used herein, unless otherwise specified or indicated from context, the term “pre-treatment” as used in accordance with the methods described herein refers to prior to administration of a treatment.

As used herein, the terms “patient” and “subject” refer to an animal, such as a mammal. In some embodiments, the patient is a human. In other embodiments, the patient is a non-human animal, such as a dog, cat, farm animal (e.g., horse, pig, or donkey), chimpanzee, or monkey. In specific embodiments, the patient is a human with lymphoma (e.g., DLBCL) in need of treatment.

### 5.2 Methods of Clustering Lymphoma Patients

In one aspect, provided herein are methods of classifying lymphoma patients, comprising (a) obtaining samples from lymphoma patients; (b) measuring the expression level of at least one gene in the samples; and (c) clustering the lymphoma patients into subgroups of patients having lymphoma using the expression level of the at least one gene in the samples. In some embodiments, the lymphoma patients are DLBCL patients

In another aspect, provided herein are methods of classifying lymphoma patients, comprising (a) measuring the expression level of at least one gene in samples from lymphoma patients; and (b) clustering the lymphoma patients into subgroups of patients having lymphoma using the expression level of the at least one gene in the samples. In certain embodiments, the method further comprises obtaining the samples from the lymphoma patients. In some embodiments, the lymphoma patients are DLBCL patients.

In some embodiments, the lymphoma is DLBCL. In some embodiments, the lymphoma is indolent B cell lymphoma. In some embodiments, the lymphoma is selected from the group consisting of follicular lymphoma, small lymphocytic lymphoma, nodal marginal zone B-cell lymphoma, lymphoplasmacytic lymphoma, anaplastic large cell lymphoma, primary cutaneous type lymphoma, mycosis fungoides, chronic lymphocytic leukemia, and mantle cell Lymphoma. In some embodiments, the lymphoma is follicular lymphoma. In some embodiments, the lymphoma is nodal marginal zone B-cell lymphoma. In some embodiments, the lymphoma is mantle cell lymphoma. In some embodiments, the lymphoma is chronic lymphocytic leukemia.

In some embodiments, the sample is obtained from a tissue of the patient comprising DLBCL cells. More detailed description of the sample (e.g., a biological sample) is described in Section 5.7 below.

In some embodiments, the DLBCL patients are newly diagnosed (nd) DLBCL patients. In some embodiments, the DLBCL patients are relapsed/refractory (r/r) DLBCL patients. In some embodiments, the DLBCL patients are newly diagnosed (nd) and relapsed/refractory (r/r) DLBCL patients.

As exemplified in Example 1 provided in Section 6.1 below, multiple patient datasets can be utilized in the classifying/clustering method. The patient datasets include but are not limited to the screening cohort from the ROBUST clinical trial consisting of 1016 patients with newly diagnosed DLBCL (see Clinical Trial No.: NCT02285062; see, e.g., Nowakowski et al., J. Clin. Onco., 2021, 39(12), 1317-1328); a set of 192 commercially-sourced newly diagnosed DLBCL patient samples which had molecular profiling but no survival data (“Commercial dataset”, when combined with the 1016-patient dataset, called the “Discovery-2 dataset”); a set of 343 ndDLBCL patients from the Molecular Epidemiology Resource (MER) (“ndMER dataset”; see Cerhan et al., Int. J. Epidermiol., 2017, 46(6):1753-1754i); a set of 928 patients from the REMoDL-B dataset (see Clinical Trial No.: NCT01324596). A subset of the Discovery-2 dataset had outcome and clinical information available. The cohort in the MER dataset has been well characterized in terms of clinical outcome and treatment. In some embodiments, the dataset is the Discovery-2 dataset. In some embodiments, the Discovery-2 dataset comprises the Commercial dataset and the ROBUST clinical trial screening dataset.

In some embodiments, measuring the gene expression levels in the samples generates a dataset of the lymphoma patients. In some embodiments, the lymphoma patients are a newly diagnosed lymphoma patient cohort. In some embodiments, the lymphoma patients are a relapsed/refractory lymphoma patient cohort.

In some embodiments, clustering the lymphoma patients into subgroups comprises using a discovery dataset and one or more replication/validation datasets. In some embodiments, the clustering step comprises a discovery dataset and a replication/validation dataset. In some embodiments, the discovery dataset is from samples from a discovery cohort. In some embodiments, the replication/validation dataset is from samples from a replication/validation cohort. In some embodiments, the discovery dataset is selected from the group consisting of Discovery-2 dataset, ndMER dataset, ROBUST dataset and REMoDL-B dataset. In some embodiments, the discovery dataset is selected from the group consisting of Discovery-2 dataset, ndMER dataset, and REMoDL-B dataset. In some embodiments, the replication/validation dataset is Discovery-2 dataset or ndMER dataset. In some embodiments, the replication/validation dataset is ndMER dataset or REMoDL-B dataset. In some embodiments, the replication/validation dataset comprises the ndMER and REMoDL-B dataset. In some embodiments, the discovery dataset is Discovery-2 dataset. In some embodiments, the discovery dataset is ndMER dataset. In some embodiments, the discovery dataset is ROBUST dataset. In some embodiments, the discovery dataset is REMoDL-B dataset. In some embodiments, the Discovery-2 dataset comprises the ROBUST dataset and the Commercial dataset. In some embodiments, the discovery dataset is a combination of one or more datasets of DLBCL patients. In some embodiments, the discovery dataset is a combination of one or more datasets described herein.

In some embodiments, clustering the lymphoma patients into subgroups comprises (i) normalizing a dataset; (ii) selecting at least one clustering feature; and (iii) applying a clustering method using the at least one clustering feature. In some embodiments, the clustering step further comprises detecting outliers in the dataset and removing the outliers. In some embodiments, the clustering step further comprises evaluating clustering results of the clustering method. In some embodiments, the clustering is an unsupervised clustering method. In some embodiments, the clustering method is a hierarchical clustering method. In some embodiments, the clustering method is a non-hierarchical method. In some embodiments, the clustering method is K means clustering method. In some embodiments, the clustering method is a partitioning method. In some embodiments, the clustering method is a fuzzy clustering method. In some embodiments, the clustering method is a density-based clustering. In some embodiments, the clustering method is a model-based clustering. In one preferred embodiment, the clustering method is iClusterPlus clustering method. In some embodiments, the clustering method is Cluster of Cluster Analysis (COCA) clustering method.

In some embodiments, a single sample normalization (ssNorm) method is used to normalize the datasets prior to analysis. The practice of performing ssNorm can benefit both immediate short-term analyses and long-term translatability of findings. In the short term, ssNorm provides a fixed normalization scheme that can apply to individual samples completely independently. There is no need to re-normalize datasets with the addition of new batches of samples, or when combining datasets for larger analyses. The ssNorm method can also implicitly perform batch correction, thereby removing gene-specific experimental effects that appear as systematic, biased differences in the raw expression space. The ssNorm method can properly align diverse datasets to a common space, showing no meaningful separation by dataset/batch when projecting into PCA space. ssNorm also allows simple translatability of any classifier or parameterization from one dataset to another—a classifier built on one ssNorm dataset can be directly applied to any other ssNorm dataset, without any need for reweighting model parameters or setting new thresholds.

In certain embodiments, DLBCL-specific housekeeping genes are used for normalization. In some embodiments, ISY1, R3HDM1, TRIM56, UBXN4, and/or WDR55 are used for normalization.

Clustering methods are sensitive to the input data, and algorithm performance can be degraded by introducing noisy or irrelevant features. Both feature selection and feature engineering approaches can be utilized to reduce the dimensionality of the dataset while maintaining a representation of relevant biological activity.

In some embodiments, one or more (e.g., one, two, three, four, or more) features are selected as the clustering features as input into the clustering method. In some embodiments, a subset of the gene expression data is selected as the clustering features. In some embodiments, the subset of the gene expression data is gene expression data of the top 50%, 45%, 40%, 35%, 30%, 25%, 20%, 15%, 10%, or 5% most expressed genes. In some embodiments, the subset of the gene expression data are gene expression data of the top 25% most expressed genes.

Some types of derived features scores that aggregate multiple biologically related genes into a single feature can also be used as clustering features. Gene Set Variation Analysis (GSVA) is a Gene Set Enrichment (GSE) method that estimates variation of pathway activity over a sample population in an unsupervised manner. GSVA provides increased power to detect subtle pathway activity changes over a sample population in comparison to corresponding methods. GSVA constitutes a starting point to build pathway-centric models of biology and GSVA can contribute to the current need of GSE methods for RNA-seq data. See Hanzelman et al., BMC Bioinformatics, 2013, 14:7. GSVA can be performed over the 50 hallmark pathway gene sets from MSigDB (Hallmark GSVA scores). See, e.g., Liberzon et al., Cell Syst., 2015, 1:417:425. GSVA can be performed over the 299 C1 set of positional cytoband signatures gene sets from MSigDB (C1 Positional GSVA scores). See, e.g., Alhamdoosh et al., F1000Research, 2017, 6:2010. GSVA can also be performed over the DLBCL-specific LM23 matrix (Cell type LM23 GSVA scores), deriving from the DCQ cellular deconvolution method. See Althoum et al., Mol. Syst. Bio., 2014, 10:720.

In some embodiments, the Hallmark GSVA scores are selected as the clustering features. In some embodiments, the C1 Positional GSVA scores are selected as the clustering features. In some embodiments, the Cell type LM23 GSVA scores are selected as the clustering features. In some embodiments, a subset of the gene expression data, Hallmark GSVA scores, C1 Positional GSVA scores, Cell type LM23 GSVA scores are selected as the matrices of clustering features.

In some embodiments, the clustering method performs clustering on each feature matrix independently and aggregates the cluster features. In some embodiments, the clustering method performs clustering aggregates the cluster features from different matrices of features and clustering across all the clustering features.

In some embodiments, the dataset is clustered into 2-20 clusters (K=2-20). In some embodiments, the dataset is clustered into 2-15 clusters (K=2-15). In some embodiments, the dataset is clustered into 2-12 clusters (K=2-12). In some embodiments, the dataset is clustered into 2-10 clusters (K=2-10). In some embodiments, the dataset is clustered into 2, 3, 4, 5, 6, 7, 8, 9, 10, 11, 12, 13, 14, 15, 16, 17, 18, 19, or 20 clusters. In some embodiments, the number of clusters is 2, 3, 4, 5, 6, 7, 8, 9, 10, 11, 12, 13, 14, 15, 16, 17, 18, 19 or 20. In some embodiments, the dataset is clustered into 7 clusters (K=7, clusters A1-A7). In some embodiments, the dataset is clustered into 8 clusters (K=8). In some embodiments, the number of cluster is 7.

In some embodiments, the clustering step further comprises evaluating clustering results of the clustering method. In some embodiments, the clustering results of each number of clusters (K) are evaluated. In some embodiments, the clustering results are evaluated using nbClust R package. In some embodiments, the clustering results are evaluated using metrics selected from the group consisting of silhouette statistic, gap statics, and percentage of variance explained by the clustering. In some embodiments, the clustering results are evaluated by the minimum cluster size. In order to have sufficient sample size in each cluster to build a downstream classifier model that could robustly identify each cluster or subgroup, the resulting clusters from each tested clustering need to have a minimum cluster size.

In some embodiments, the clustering method is iClusterPlus clustering method and the number of cluster is 7.

In some embodiments, the clustering method is iClusterPlus clustering method, a subset of the gene expression data, Hallmark GSVA scores, C1 Positional GSVA scores, Cell type LM23 GSVA scores are selected as the matrices of clustering features, and the number of cluster is 7 (clusters A1-A7).

In some embodiments, for one or more of the subgroups, there are patients that give low confidence level data for a specific subgroup in view of the selected clustering classifiers. In some embodiments, the patients that give low confidence level clustering data are filtered. In some embodiments, the patients that give low confidence level clustering data are excluded from the subgroup. In some embodiments, the patients that give low confidence level clustering data are excluded from assignment to a subgroup. In some embodiments, patients that give low confidence level clustering data are excluded from subgroup A7.

In some embodiments, the method further comprises setting a threshold confidence level for one or more of the subgroups to exclude patients that give lower confidence level clustering data from the one or more subgroup(s). In some embodiments, patients that give low confidence level clustering data are excluded from subgroup A7.

In some embodiments, the method of clustering lymphoma patients further comprises (i) identifying at least one cluster classifier by training a classifier model using the clustering results of a discovery dataset, (ii) applying the at least one cluster classifier to a replication/validation dataset to classify the replication/validation dataset, (iii) clustering the replication/validation dataset using the clustering method; and (iv) comparing the classification results of the replication/validation dataset using the at least one cluster classifier with the clustering results of the replication/validation dataset using the clustering method. In some embodiments the model is a grouped multinomial generalized linear model (GLM). In some embodiments, the model is a binary generalized linear model (GLM). In some embodiments, the model is GLM using least absolute shrinkage and selection operator (LASSO). In some embodiments, if the classification results of the replication/validation dataset using the at least one cluster classifier is similar to the clustering results of the replication/validation dataset using the clustering method, it indicates that the at least one cluster classifier is effective for classifying the replication/validation dataset.

In some embodiments, the method of clustering lymphoma patients further comprises (i) identifying at least one cluster classifier by training a classifier model using the clustering results of a discovery dataset, and (ii) applying the at least one cluster classifier to a replication/validation dataset to classify the replication/validation dataset. In some embodiments, the model is a grouped multinomial generalized linear model (GLM). In some embodiments, the model is a binary generalized linear model (GLM). In some embodiments, the model is GLM using least absolute shrinkage and selection operator (LASSO).

In some embodiments, the age of the lymphoma patient is 30 years or older, 35 years or older, 40 years or older, 45 years or older, 50 years or older, or 55 years or old, or 60 years or older, or 65 years or older, or 70 years or older at baseline. In some embodiments, the age of the lymphoma patient is 70 years or older. In some embodiments, the age of the lymphoma patient is 60 years or older. In some embodiments, the age of the lymphoma patient is between 30 years and 35 years, between 35 years and 40 years, between 40 years and 45 years, between 45 years and 50 years, between 50 years and 55 years, between 55 years and 60 years, between 60 years and 65 years, or between 65 years and 70 years.

In certain embodiments, the at least one gene is selected from the genes of Table 1. In certain embodiments, the at least one gene comprises one, two, three, four, five, or more genes of Table 1. In certain embodiments, the at least one gene comprises all genes of Table 1.

In some embodiments, the expression level of at least one gene in the discovery dataset is used in training the classifier model. In some embodiments, one, two, three, four, five or more of the genes of Table 1 are used in training the classifier model. In some embodiments, all genes of Table 1 are used in training the classifier model.

In some embodiments, the classifier model comprises one, two, three, four, five, or more of the genes of Table 1. In some embodiments, the classifier model comprises all genes of Table 1. In some embodiments, the expression levels of all genes of Table 1 are determined for clustering. In some embodiments, the expression levels of all genes of Table 1 are determined and compared for determining whether the lymphoma patient is responsive to a cancer treatment.

TABLE 1
List of Genes Utilized in the Resulting Grouped
Multinomial Generalized Linear Model (GLM) Model

| | | | | |
|---|---|---|---|---|
| ABHD10 | ACO1 | ACTN4 | AGRP | AKAP13 |
| ALDH1A1 | ALG13 | AMT | ANKZF1 | AOAH |
| AP1G2 | AP3S1 | APRT | ARG1 | ARHGDIA |
| ARHGEF7 | ART4 | ASH1L | ATIC | ATP6V1G2 |
| ATP9B | BAZ2A | BLNK | BPNT1 | BRIP1 |
| BTF3 | BUB3 | C1QBP | C2 | CACUL1 |
| CADPS | CAPZB | CARD11 | CBX5 | CCDC136 |
| CCR2 | CCT7 | CCT8 | CD37 | CD46 |
| CDC25A | CDK12 | CENPW | CEP85L | CEP97 |
| CFH | CHD2 | CHI3L1 | CHKA | CIB1 |
| CKAP2 | CLCN7 | CLEC7A | CLIC1 | CLK1 |
| CMSS1 | COG7 | COL1A2 | COL4A3 | CORO1A |
| COX6C | CPD | CRBN | CSF1 | CUEDC2 |
| CUX1 | CXCL10 | DDHD1 | DDX58 | DIMT1 |
| DNAJA3 | DNAJC10 | DNMT1 | DYNLT1 | E2F1 |
| EBAG9 | EIF1AX | EIF2B5 | EIF3I | EIF5A |
| ELF1 | ELMO1 | EMP3 | ENO1 | EPS15 |
| ERGIC2 | ERH | ESD | EYS | FBXO46 |
| FLNA | FOXP1 | FPR1 | FUBP1 | FUS |
| GALM | GAPDH | GATM | GBP5 | GIMAP4 |
| GJD3 | GLUL | GNA13 | GNB2 | GNS |
| GPR82 | GPX1 | GRIP1 | HAMP | HEXIM1 |
| HNMT | HNRNPA2B1 | HNRNPU | HSD11B1L | HSP90AA1 |
| HSPD1 | HSPE1 | IDS | IFI30 | IFITM3 |
| IKZF1 | IL24 | IMPDH2 | IST1 | ITGB2 |
| JAK1 | KIF14 | KIF4A | KLHL14 | KLHL23 |
| LAP3 | LATS1 | LMO4 | LONP2 | LPAR6 |
| LRCH4 | LRRC37A2 | LRRC37A3 | LRRC59 | LY9 |
| LYRM1 | MAP3K14 | MAP4K2 | MAPK14 | MARS2 |
| MAT2A | MAX | MCL1 | MCM4 | MGAT4A |
| MRPL43 | MRPS15 | MRPS9 | MSH6 | MVB12A |
| MVB12B | MVP | MYBL2 | MYOF | NACA |
| NCAPG2 | NCBP2 | NCL | NFE2L2 | NFKBIA |
| NRXN1 | NUDT21 | ORC6 | P2RX7 | PAICS |
| PARG | PARP9 | PAX5 | PCNA | PDE9A |
| PFKL | PGAM1 | PIF1 | PILRB | PKIA |
| PKM | PKN2 | PLEKHF2 | PLK1 | PMM2 |
| PNP | POLA1 | POLR2E | POM121 | PPA1 |
| PPIA | PPP1R9B | PRDM15 | PRDX5 | PRKCB |
| PRKCH | PRMT1 | PRPF4 | PRPF6 | PRRC2C |
| PSMC1 | PSMD13 | PSME2 | PTBP3 | PTENP1 |
| PTPRC | R3HDM1 | RAB32 | RAMP3 | RANBP2 |
| RBM3 | RBM33 | RBP1 | RFC1 | RNASEL |
| RNF38 | RPL30 | RPL32P3 | RPRD2 | RPS3 |
| RPS4X | RRP9 | S100A11 | S100Z | S1PR2 |
| SAAL1 | SBNO1 | SCAMP2 | SCARB1 | SCARB2 |
| SDCBP | SELPLG | SENP7 | SERPINA3 | SERPINB1 |
| SF1 | SF3A1 | SFPQ | SFXN3 | SH3BGRL3 |
| SH3BP1 | SLAMF8 | SLC35D1 | SMARCA4 | SMARCC1 |
| SMG5 | SNORA21 | SNORA71B | SNORD104 | SNRPA |
| SNRPD1 | SNTA1 | SNX29 | SOBP | SOGA1 |
| SP140 | SP3 | SPEN | SRGN | SRSF6 |
| STAG3 | STK4 | STMN1 | SUMO2 | SYNJ2 |
| SYPL1 | SYTL3 | TAGLN2 | TAP2 | TARDBP |
| TBCA | TGDS | TGOLN2 | THRAP3 | TIMM10 |
| TLK1 | TLN1 | TMBIM4 | TMEM223 | TNFRSF1A |
| TONSL | TP53INP1 | TPM3 | TRA2B | TRAM1 |
| TRAPPC12 | TRIOBP | TRIP13 | TRMT1L | TRNT1 |
| TSPYL2 | TSSK4 | TUBA1B | TUBA1C | TWIST1 |
| UBE2B | UBE2D2 | UBE2G1 | UXT | VASH1 |
| VAV1 | VDAC1 | WEE1 | XRCC6 | YTHDC1 |
| YWHAE | ZBED5 | ZBTB37 | ZFAND4 | ZFAND5 |
| ZMAT1 | ZNF101 | ZNF107 | ZNF146 | ZNF207 |
| ZNF318 | ZNF367 | ZNF480 | ZWINT | |

In some embodiments, the method provided herein comprises at least one gene (e.g., one, two, three, four, five, or more) selected from the group consisting of or all genes from the group consisting of ABHD10, ACO1, ACTN4, AGRP, AKAP13, ALDH1A1, ALG13, AMT, ANKZF1, AOAH, AP1G2, AP3S1, APRT, ARG1, ARHGDIA, ARHGEF7, ART4, ASH1L, ATIC, ATP6V1G2, ATP9B, BAZ2A, BLNK, BPNT1, BRIP1, BTF3, BUB3, C1QBP, C2, CACUL1, CADPS, CAPZB CARD11, CBX5, CCDC136, CCR2, CCT7, CCT8, CD37, CD46, CDC25A, CDK12, CENPW, CEP85L, CEP97, CFH, CHD2, CHI3L1, CHKA, CIB1, CKAP2, CLCN7, CLEC7A, CLIC1, CLK1, CMSS1, COG7, COL1A2, COL4A3, CORO1A, COX6C, CPD, CRBN, CSF1, CUEDC2, CUX1, CXCL10, DDHD1, DDX58, DIMT1, DNAJA3, DNAJC10, DNMT1, DYNLT1, E2F1, EBAG9, EIF1AX, EIF2B5, EIF3I, EIF5A, ELF1, ELMO1, EMP3, ENO1, EPS15, ERGIC2, ERH ESD, EYS, FBXO46, FLNA, FOXP1, FPR1, FUBP1, FUS, GALM GAPDH, GATM, GBP5, GIMAP4, GJD3, GLUL, GNA13 GNB2, GNS, GPR82, GPX1, GRIP1, HAMP, HEXIM1, HNMT, HNRNPA2B1, HNRNPU, HSD11B1L, HSP90AA1, HSPD1, HSPE1, IDS, IFI30, IFITM3, IKZF1, IL24, IMPDH2, IST1, ITGB2, JAK1, KIF14, KIF4A, KLHL14, KLHL23, LAP3, LATS1, LMO4, LONP2, LPAR6, LRCH4, LRRC37A2, LRRC37A3, LRRC59, LY9, LYRM1, MAP3K14, MAP4K2, MAPK14, MARS2, MAT2A, MAX, MCL1, MCM4, MGAT4A, MRPL43, MRPS15, MRPS9, MSH6, MVB12A, MVB12B, MVP, MYBL2, MYOF, NACA, NCAPG2, NCBP2, NCL, NFE2L2, NFKBIA, NRXN1, NUDT21, ORC6, P2RX7, PAICS, PARG, PARP9, PAX5, PCNA, PDE9A, PFKL, PGAM1, PIF1, PILRB, PKIA, PKM, PKN2, PLEKHF2, PLK1 PMM2, PNP, POLA1, POLR2E, POM121, PPA1, PPIA, PPP1R9B, PRDM15, PRDX5, PRKCB, PRKCH, PRMT1, PRPF4, RPF6, PRRC2C, PSMC1, PSMD13, PSME2, PTBP3, PTENP1, PTPRC, R3HDM1, RAB32, RAMP3, RANBP2, RBM3, RBM33, RBP1, RFC1, RNASEL, RNF38, RPL30, RPL32P3, RPRD2, RPS3, RPS4X, RRP9, S100A11, S100Z, S1PR2, SAAL1, SBNO1, SCAMP2, SCARB1, SCARB2, SDCBP, SELPLG, SENP7, SERPINA3, SERPINB1, SF1, SF3A1, SFPQ, SFXN3, SH3BGRL3, SH3BP1, SLAMF8, SLC35D1, SMARCA4, SMARCC1, SMG5, SNORA21, SNORA71B, SNORD104, SNRPA, SNRPD1, SNTA1, SNX29, SOBP, SOGA1, SP140, SP3, SPEN, SRGN, SRSF6, STAG3, STK4, STMN1, SUMO2, SYNJ2, SYPL1, SYTL3, TAGLN2, TAP2, TARDBP, TBCA, TGDS, TGOLN2, THRAP3, TIMM10, TLK1, TLN1, TMBIM4, TMEM223, TNFRSF1A, TONSL, TP53INP1, TPM3, TRA2B, TRAM1, TRAPPC12, TRIOBP, TRIP13, TRMT1L, TRNT1, TSPYL2, TSSK4, TUBA1B, TUBA1C, TWIST1, UBE2B, UBE2D2, UBE2G1, UXT, VASH1, VAV1, VDAC1, WEE1, XRCC6, YTHDC1, YWHAE, ZBED5, ZBTB37, ZFAND4, ZFAND5, ZMAT1, ZNF101, ZNF107, ZNF146, ZNF207, ZNF318, ZNF367, ZNF480, and ZWINT.

### 5.3 Methods of Predicting Responsiveness of Lymphoma Patient and Methods of Treating

In another aspect, provided herein are methods for predicting the responsiveness of a lymphoma patient to a cancer treatment comprising: (a) obtaining reference biological samples from each patient in a reference patient group comprising reference patients having a lymphoma; (b) clustering or classifying the reference patient group into subgroups of patients using gene expression levels in the reference biological samples; (c) obtaining a biological sample from the lymphoma patient; (d) determining to which subgroup the lymphoma patient belongs using gene expression levels in the biological sample from the lymphoma patient; and (e) predicting the responsiveness of the lymphoma patient to a first cancer treatment.

In another aspect, provided herein are methods for predicting the responsiveness of a lymphoma patient to a cancer treatment comprising: (a) clustering reference lymphoma patients in a reference patient group into subgroups using the expression level of at least one gene in reference biological samples of the reference lymphoma patients; (b) determining a subgroup to which the lymphoma patient belongs based on the expression level of the at least one gene in a biological sample from the lymphoma patient; and (c) predicting the responsiveness of the lymphoma patient to a first cancer treatment based on the subgroup of the lymphoma patient. In certain embodiments, the method further comprises obtaining the reference biological samples from the reference lymphoma patients in the reference patient group. In certain embodiments, the method further comprises obtaining the biological sample from the lymphoma patient.

In some embodiments, the method further comprises administering to the lymphoma patient a second cancer treatment.

In some embodiments, the sample is obtained from a tissue of the subject comprising DLBCL cells. More detailed description of the sample (or biological sample) is provided in Section 5.7 below.

In some embodiments, the lymphoma patient is DLBCL patients. In some embodiments, the DLBCL patients are newly diagnosed (nd) and relapsed/refractory (r/r) DLBCL patients. In some embodiments, the DLBCL patients are newly diagnosed (nd) DLBCL patients. In some embodiments, the DLBCL patients are relapsed/refractory (r/r) DLBCL patients.

In some embodiments, classifying or clustering the reference patient group comprises generating clustering information defining relationships between the expression level of at least one gene in the reference biological samples; and rearranging heat map representation based on the clustering information. In some embodiments, classifying or clustering the reference patient group comprises using the clustering method described herein in Section 5.2.

In some embodiments, the clustering step comprises a discovery dataset and at least one replication/validation dataset. In some embodiments, the clustering step comprises a discovery dataset and a replication/validation dataset. In some embodiments, the discovery dataset is from samples from a discovery cohort. In some embodiments, the replication/validation dataset is from sample from a replication/validation cohort. In some embodiments, the discovery dataset is selected from the group consisting of Discovery-2 dataset, ndMER dataset, ROBUST dataset and REMoDL-B dataset. In some embodiments, the replication/validation dataset is selected from the group consisting of Commercial dataset, ndMER dataset, ROBUST dataset and REMoDL-B dataset. In some embodiments, the discovery dataset is Discovery-2 dataset. In some embodiments, the Discovery-2 dataset comprises Commercial dataset. In some embodiments, the Discovery-2 dataset comprises ROBUST dataset. In some embodiments, the Discovery-2 dataset comprises Commercial dataset and ROBUST dataset. In some embodiments, the discovery dataset is ndMER dataset. In some embodiments, the discovery dataset is REMoDL-B dataset.

In some embodiments, the replication/validation dataset is selected from the group consisting of Discovery-2 dataset, ndMER dataset, ROBUST dataset, and REMoDL-B dataset. In some embodiments, the replication/validation dataset is selected from the group consisting of commercial dataset, ndMER dataset, ROBUST dataset, and REMoDL-B dataset. In some embodiments, the replication/validation dataset is selected from the group consisting of ndMER dataset, ROBUST dataset, and REMoDL-B dataset. In some embodiments, the replication/validation dataset is ndMER dataset or REMoDL-B dataset. In some embodiments, the replication/validation dataset is ndMER dataset. In some embodiments, the replication/validation dataset is REMoDL-B dataset. In some embodiments, the replication/validation dataset is ndMER dataset and REMoDL-B dataset.

In some embodiments, the clustering step comprises (i) normalizing a dataset; (ii) selecting at least one clustering feature; and (iii) applying a clustering method using the at least one clustering feature. In some embodiments, the clustering step further comprises detecting outliers in the dataset and removing the outliers. In some embodiments, the clustering step further comprises evaluating clustering results of the clustering method. In some embodiments, the clustering is an unsupervised clustering method. In some embodiments, the clustering method is a hierarchical clustering method. In some embodiments, the clustering method is a non-hierarchical method. In some embodiments, the clustering method is K means clustering method. In some embodiments, the clustering method is a partitioning method. In some embodiments, the clustering method is a fuzzy clustering method. In some embodiments, the clustering method is a density-based clustering. In some embodiments, the clustering method is a model-based clustering. In some embodiments, the clustering method is iClusterPlus clustering method. In some embodiments, the clustering method is Cluster of Cluster Analysis (COCA) clustering method. In some embodiments, a single sample normalization (ssNorm) method is used to normalize the datasets prior to analysis.

In certain embodiments, DLBCL-specific housekeeping genes are used for normalization. In some embodiments, ISY1, R3HDM1, TRIM56, UBXN4, and WDR55 are used for normalization.

In some embodiments, one, two, three, four, or more features are selected as the clustering features as input into the clustering method. In some embodiments, a subset of the gene expression data is selected as the clustering features. In some embodiments, the subset of the gene expression data is gene expression data of the top 50%, 45%, 40%, 35%, 30%, 25%, 20%, 15%, 10%, or 5% most expressed genes. In some embodiments, the subset of the gene expression data is gene expression data of the top 25% most expressed genes.

In some embodiments, the Hallmark GSVA scores are selected as the clustering features. In some embodiments, the C1 Positional GSVA scores are selected as the clustering features. In some embodiments, the Cell type LM23 GSVA scores are selected as the clustering features. In some embodiments, a subset of the gene expression data, Hallmark GSVA scores, C1 Positional GSVA scores, Cell type LM23 GSVA scores are selected as the matrices of clustering features.

In some embodiments, the clustering method performs clustering on each feature matrix independently and aggregates the cluster features. In some embodiments, the clustering method performs clustering that aggregates the cluster features from different matrices of features and clustering across all the clustering features.

In some embodiments, the dataset is clustered into 2-20 clusters (K=2-20). In some embodiments, the dataset is clustered into 2-15 clusters (K=2-15). In some embodiments, the dataset is clustered into 2-12 clusters (K=2-12). In some embodiments, the dataset is clustered into 2-10 clusters (K=2-10). In some embodiments, the dataset is clustered into 2, 3, 4, 5, 6, 7, 8, 9, 10, 11, 12, 13, 14, 15, 16, 17, 18, 19, or 20 clusters. In some embodiments, the dataset is clustered into 8 clusters (K=8). In some embodiments, the dataset is clustered into 7 clusters (K=7, cluster A1-A7). In some embodiments, the number of cluster is 7.

In some embodiments, the clustering method is iClusterPlus clustering method and the number of cluster is 7.

In some embodiments, the clustering method is iClusterPlus clustering method, a subset of the gene expression data, Hallmark GSVA scores, C1 Positional GSVA scores, Cell type LM23 GSVA scores are selected as the matrices of clustering features, and the number of cluster is 7 (clusters A1-A7).

The terms “clusters” and “subgroups” are used interchangeably throughout the present disclosure.

In some embodiments, the reference patients in the reference patient group are clustered into 2-20 subgroups (K=2-20). In some embodiments, the reference patients in the reference patient group are clustered into 2-15 subgroups (K=2-15). In some embodiments, the reference patients in the reference patient group are clustered into 2-12 subgroups (K=2-12). In some embodiments, the reference patients in the reference patient group are clustered into 2-10 subgroups (K=2-10). In some embodiments, the reference patients in the reference patient group are clustered into 2, 3, 4, 5, 6, 7, 8, 9, 10, 11, 12, 13, 14, 15, 16, 17, 18, 19, or 20 subgroups. In some embodiments, the reference patients in the reference patient group are clustered into 8 groups (K=8). In some embodiments, the reference patients in the reference patient group are clustered into 7 groups (K=7, cluster A1-A7).

In some embodiments, the clustering step further comprises evaluating clustering results of the clustering method. In some embodiments, the clustering results of each number of clusters (K) are evaluated. In some embodiments, the clustering results are evaluated using nbClust R package. In some embodiments, the clustering results are evaluated using metrics selected from the group consisting of silhouette statistic, gap statics, and percentage of variance explained by the clustering. In some embodiments, the clustering results are evaluated by the minimum cluster size. In order to have sufficient sample size in each cluster to build a downstream classifier model that could robustly identify each cluster or subgroup, the resulting clusters from each tested clustering need to have a minimum cluster size.

In some embodiments, for at least one of the subgroups, there are patients that give low confidence level clustering data for a specific subgroup in view of the selected clustering classifiers. In some embodiments, the patients that give low confidence level clustering data are filtered. In some embodiments, the patients that give low confidence level clustering data are excluded from the subgroup. In some embodiments, patients that give low confidence level clustering data are excluded from subgroup A7.

In some embodiments, the method further comprises setting a threshold confidence level for at least one of the subgroups to exclude patients that give lower confidence level from the at least one subgroup. In some embodiments, patients that give low confidence level clustering data are excluded from subgroup A7.

In some embodiments, the method of for predicting the responsiveness of a lymphoma patient to a cancer treatment further comprises (i) identifying at least one cluster classifier by training a classifier model using the clustering results of a discovery dataset, (ii) applying the at least one cluster classifier to a replication/validation dataset to classify the replication/validation dataset, (iii) clustering the replication/validation dataset using the clustering method, and (iv) comparing the classification results of the replication/validation dataset using the cluster at least one classifier with the clustering results of the replication/validation dataset using the clustering method. In some embodiments, the model is a grouped multinomial generalized linear model (GLM). In some embodiments, the model is GLM using least absolute shrinkage and selection operator (LASSO). In some embodiments, if the classification results of the replication/validation dataset using the at least one cluster classifier is similar to the clustering results of the replication/validation dataset using the clustering method, it indicates that the cluster classifier(s) is effective for classifying the replication/validation dataset.

In some embodiments, the method for predicting the responsiveness of a lymphoma patient to a cancer treatment further comprises (i) identifying at least cluster classifier by training a classifier model using the clustering results of a discovery dataset, and (ii) applying the at least one cluster classifier to a replication/validation dataset to classify the replication/validation dataset. In some embodiments, the model is a grouped multinomial generalized linear model (GLM). In some embodiments, the model is GLM using least absolute shrinkage and selection operator (LASSO).

In certain embodiments, the at least one gene is selected from the genes of Table 1. In certain embodiments, the at least one gene comprises one, two, three, four, five, or more genes of Table 1. In certain embodiments, the at least one gene comprises all genes of Table 1.

In some embodiments, the expression levels of at least one gene in the discovery dataset are used in training the classifier model. In some embodiments, one, two, three, four, five or more of the genes of Table 1 are used in training the classifier model. In some embodiments, all genes of Table 1 are used in training the classifier model.

In some embodiments, the classifier model comprises one, two, three, four, five, or more of the genes of Table 1. In some embodiments, the classifier model comprises all genes identified in Table 1.

In some embodiments, the determining step applies the clustering method to determine to which subgroup the lymphoma patient belongs to using gene expression levels in the biological sample from the lymphoma patient.

In some embodiments, the predicting step applies the trained classifier model to predict the responsiveness of the lymphoma patient to a first cancer treatment. In some embodiments, the predicting step applies the trained GLM model to predict the responsiveness of the lymphoma patient to a first cancer treatment.

In some embodiments, the lymphoma is diffuse large B-cell lymphoma (DLBCL). In some embodiments, the lymphoma is indolent B cell lymphoma. In some embodiments, the lymphoma is selected from the group consisting of follicular lymphoma, small lymphocytic lymphoma, nodal marginal zone B-cell lymphoma, lymphoplasmacytic lymphoma, anaplastic large cell lymphoma, primary cutaneous type lymphoma, mycosis fungoides, chronic lymphocytic leukemia, and mantle cell Lymphoma. In some embodiments, the lymphoma is follicular lymphoma. In some embodiments, the lymphoma is nodal marginal zone B-cell lymphoma. In some embodiments, the lymphoma is mantle cell lymphoma. In some embodiments, the lymphoma is chronic lymphocytic leukemia.

In some embodiments, the first cancer treatment is a combination treatment with rituximab (Rituxan), cyclophosphamide, doxorubicin, vincristine, and prednisone (R-CHOP).

In some embodiments, the second cancer treatment is R-CHOP. In some embodiments, the second cancer treatment is not R-CHOP.

In some embodiments, the second cancer treatment is a bromodomain and extra-terminal (BET) inhibitor, or a cyclin dependent kinase (CDK) inhibitor.

Bromodomains (BDs) are protein modules of ˜110 amino acids that recognize acetylated lysine in histones and other proteins. The BET family is a subset of 46 bromodomain-containing proteins found only in the human genome. BET proteins are composed of four proteins, namely bromodomain-containing protein 2 (BRD2), BRD3, BRD4 and bromodomain testis-specific protein (BRDT). See Cochran et al., Nature Reviews Drug Discovery, 2019, 18:609-628; Duan et al., MedChemComm, 2018, 9:1779-1802. Tool compounds showing robust preclinical activity have led to advances in the development of BET inhibitors, primarily in oncology indications including, for example, DLBCL. As the chemical probes described for other BD targets, clinical BET inhibitors are acetyl lysine mimetics with a heterocyclic core that occupies the BD pocket15. Deregulation of BET proteins, in particular BRD4, has been implicated in the development of diverse diseases, especially cancers. See Duan et al., MedChemComm, 2018, 9:1779-1802.

In some embodiments, the BET inhibitor is selected from the group consisting of OTX015, MK-8628, CPI-0610, BMS-986158, ZEN003694, GSK2820151, GSK525762, INCB054329, INCB057643, ODM-207, RO6870810, BAY1238097, CC-90010, AZD5153, FT-1101, ABBV-075, ABBV-744, SF1126, GS-5829, and CPI-0610.

In some embodiments, the BET inhibitor is a bromodomain-containing protein 4 (BRD4) inhibitor. In some embodiments, the BRD4 inhibitor is selected from the group consisting of OTX015, TEN-010, GSK525762, CPI-0610, I-BET151, PLX51107, INCB0543294, ABBV-075, BI 894999, BMS-986158, and AZD5153.

Cyclin-dependent kinases (CDKs) are a family of serine-threonine kinases that are identified as gene products involved in cell cycle control. A close cooperation between CDKs, cyclins and cells contain endogenous inhibitors (CKIs) is necessary for orderly cell cycle progressions under regulation. Mammalian CDKs, cyclins and CKIs play important roles in other biological processes including, for example, transcriptional regulation, epigenetics, DNA damage response and repair (DDR), stemness, metabolism and angiogenesis among others. See Sanchez-Martinez et al., Bioorg. Med. Chem. Lett., 2019, 29:126637.

In some embodiments, the CDK inhibitor is selected from the group consisting of PD-0332991 (Ibrance® or Palbociclib), LEE011 (Ribociclib), LY2835219 (Verzenio® or Abemaciclib), G1T28 (Trilaciclib), G1T38 (Lerociclib), SHR-6390, Flavopiridol (Alvocidib), PHA848125 (Milciclib), BCD-115, MM-D37K, PF-06873600, TG-02 (SB-1317 or Zoriraciclib), C7001 (ICEC 0942), BEY-1107, XZP-3287 (Birociclib), BPI-16350, FCN-437, CYC-065, R-Roscovitine (CY-202 or Seliciclib), AT-7519, AGM-130 (Inditinib), FN-1501, SY-1365, AZD-4573, TP-1287, P-1446A-05 (Voruciclib), BAY-1251152, SCH-727965 (MK-765 or Dinaciclib), BEBT-209, TQB-3616, BAY-1000394 (Roniciclib), BAY-1143572 (Atuveciclib), and AGM-925 (FLX-925).

### 5.3.1. Double Hit Signature (DHITsig) Classifier

It has been well described in literature that there exists a subset of high-risk DLBCL patients who are hallmarked by a so-called “double hit” (DHIT) translocation in MYC and either BCL2 or BCL6. Patients who are DHIT+ tend to have worse survival than DHIT− patients, and these patients are typically GCB with MYC+BCL2 translocations. DHIT status has been measured using DNA sequencing or FISH probes to determine translocation status.

Ennishi et al. derived a gene expression signature that reflects DHIT status, capturing a broader segment of the population that has differential clinical outcome. See Ennishi et al., J. Clin. Oncol., 2018, 37:190-201. Using the 104 genes, parameterization, and methodology described in the manuscript, the DHITsig method was adapted and implemented for use in the single-sample normalized RNAseq space.

The Ennishi method for calculating the DHITsig score is a variable importance-weighted sum of log likelihood ratios. The likelihood function is calculated by assuming a Gaussian mixture model of gene expression that defines two normal distributions of expression for DHITsig+ and DHITsig− samples for each signature gene. The gene list and variable weights from Ennishi et al. were used, along with the mixture model distribution parameters as derived from their DESeq2-processed dataset.

### 5.3.2. Identifying Biological Features of Clusters of Patients and Treating

In some embodiments, the reference patients in the reference patient group are clustered into subgroups A1-A7; and wherein: (i) subgroup A1 comprises about 50% to about 60% patients having germinal center B-cell-like (GCB) DLBCL, about 30% to about 40% patients having activated B-cell like (ABC) DLBCL, about 10% to about 20% patients who are TME+ DLBCL patients, and about 30% to about 40% patients who are DHITsig+ DLBCL patients; (ii) subgroup A2 comprises about 80% to about 90% patients having GCB DLBCL, about 0% to about 5% patients having ABC DLBCL, about 15% to about 25% patients who are TME+ DLBCL patients, and about 25% to about 35% patients who are DHITsig+ DLBCL patients; (iii) subgroup A3 comprises about 40% to about 55% patients having GCB DLBCL, about 30% to about 45% patients having ABC DLBCL, about 40% to about 50% patients who are TME+ DLBCL patients, and about 20% to about 30% patients who are DHITsig+ DLBCL patients; (iv) subgroup A4 comprises about 25% to about 35% patients having GCB DLBCL, about 40% to about 50% patients having ABC DLBCL, about 30% to about 40% patients who are TME+ DLBCL patients, and about 10% to about 20% patients who are DHITsig+ DLBCL patients; (v) subgroup A5 comprises about 20% to about 40% patients having GCB DLBCL, about 45% to about 65% patients having ABC DLBCL, about 30% to about 40% patients who are TME+ DLBCL patients, and about 0% to about 10% patients who are DHITsig+ DLBCL patients; (vi) subgroup A6 comprises about 30% to about 40% patients having GCB DLBCL, about 40% to about 50% patients having ABC DLBCL, about 75% to about 95% patients who are TME+ DLBCL patients, and about 0% to about 10% patients who are DHITsig+ DLBCL patients; and (vii) subgroup A7 comprises about 0% to about 10% patients having GCB DLBCL, about 80% to about 90% patients having ABC DLBCL, about 0% to about 10% patients who are TME+ DLBCL patients, and about 5% to about 15% patients who are DHITsig+ DLBCL patients.

In some embodiments, when the lymphoma patient is determined to belong to subgroup A1, the method comprises predicting that the patient is not likely to be responsive to the first cancer treatment. In some embodiments, when the lymphoma patient is determined to belong to subgroup A2, the method comprises predicting that the patient is not likely to be responsive to the first cancer treatment. In some embodiments, when the lymphoma patient is determined to belong to subgroup A3, the method comprises predicting that the patient is not likely to be responsive to the first cancer treatment. In some embodiments, when the lymphoma patient is determined to belong to subgroup A4, the method comprises predicting that the patient is not likely to be responsive to the first cancer treatment. In some embodiments, when the lymphoma patient is determined to belong to subgroup A5, the method comprises predicting that the patient is not likely to be responsive to the first cancer treatment. In some embodiments, when the lymphoma patient is determined to belong to subgroup A6, the method comprises predicting that the patient is not likely to be responsive to the first cancer treatment. In some embodiments, when the lymphoma patient is determined to belong to subgroup A7, the method comprises predicting that the patient is not likely to be responsive to the first cancer treatment.

In some embodiments, when the lymphoma patient is determined to belong to activated B-cell like (ABC) lymphoma patient in subgroup A7, the method comprises predicting that the patient is not likely to be responsive to the first cancer treatment.

In some embodiments, when the lymphoma patient is determined to belong to any of the subgroup of patients that is predicted to be not likely to be responsive to the first cancer treatment, the lymphoma patient is treated with a second cancer treatment. In some embodiments, the second cancer treatment is a BET inhibitor or a CDK inhibitor. In some embodiments, the second cancer treatment is a CDK inhibitor. In some embodiments, the second cancer treatment is a BET inhibitor.

In some embodiments, when the lymphoma patient is determined to belong to subgroup A7, the second cancer treatment is a BET inhibitor or a CDK inhibitor. In some embodiments, when the lymphoma patient is determined to belong to subgroup A7, the second cancer treatment is a CDK inhibitor. In some embodiments, when the lymphoma patient is determined to belong to subgroup A7, the second cancer treatment is a BET inhibitor.

In some embodiments, when the lymphoma patient is determined to belong to any of the subgroup of patients that is predicted to be not likely to be responsive to the first cancer treatment, the lymphoma patient is treated with a BCL2 inhibitor. In some embodiments, when the lymphoma patient is determined to belong to any of the subgroup of patients that is predicted to be not likely to be responsive to the first cancer treatment, the lymphoma patient is treated with an agent that increases FAS expression. In some embodiments, when the lymphoma patient is determined to belong to any of the subgroup of patients that is predicted to be not likely to be responsive to the first cancer treatment, the lymphoma patient is treated with an inhibitor of human leukocyte antigen (HLA) genes. In some embodiments, the second cancer treatment is an inhibitor of HLA-A. In some embodiments, the second cancer treatment is an inhibitor of HLA-B. In some embodiments, the second cancer treatment is an inhibitor of HLA-C. In some embodiments, the second cancer treatment is an inhibitor of HLA-E. In some embodiments, the second cancer treatment is an inhibitor of HLA-F. In some embodiments, when the lymphoma patient is determined to belong to any of the subgroup of patients that is predicted to be not likely to be responsive to the first cancer treatment, the lymphoma patient is treated with a CD47 treatment. In some embodiments, when the lymphoma patient is determined to belong to any of the subgroup of patients that is predicted to be not likely to be responsive to the first cancer treatment, the lymphoma patient is treated with an IDO inhibitor or an agent that depletes regulatory T cells. In some embodiments, the second cancer treatment is an IDO inhibitor. In some embodiments, the second cancer treatment is an agent that depletes regulatory T cells. In some embodiments, when the lymphoma patient is determined to belong to any of the subgroup of patients that is predicted to be not likely to be responsive to the first cancer treatment, the lymphoma patient is treated with a histone deacetylase (HDAC) inhibitor. In some embodiments, when the lymphoma patient is determined to belong to any of the subgroup of patients that is predicted to be not likely to be responsive to the first cancer treatment, the lymphoma patient is treated with a galectin-3 (Gal3) inhibitor.

In some embodiments, the lymphoma patient belongs to a subgroup based on the mutational data of at least one feature listed in Table 6 or Table 7.

In another aspect, provided herein are methods of predicting the responsiveness of lymphoma patient to a cancer treatment comprising: (a) obtaining a first biological sample from a first patient having a lymphoma; (b) determining the level of expression of one, two, three, four, five or more of the genes identified in Table 1; (c) comparing the level of expression of the one, two, three, four, five or more of the genes identified in Table 1 in the first biological sample with the level of expression of the same genes in a second biological sample(s) from a second patient(s), wherein the second lymphoma patient(s) is responsive to the cancer treatment, and wherein the similar expression of the one, two, three, four, five or more of the genes in the first biological sample relative to the level of expression of the one, two, three, four, five or more of the genes in the second biological sample(s) indicates that the lymphoma in the first patient will be responsive to treatment with the cancer treatment.

In another aspect, provided herein are methods for predicting the responsiveness of a lymphoma patient to a cancer treatment comprising: (a) obtaining a first biological sample from a first lymphoma patient; (b) determining the expression of the genes or a certain subset of genes set forth in Table 1 or any combination thereof in the first biological sample; and (c) comparing the gene expression profile of the genes or subset of genes in the first biological sample to (i) the gene expression profile of the genes or subset of genes in biological samples from lymphoma patients which are responsive to the drug and (ii) the gene expression of the genes or subset of genes in biological samples from lymphoma patients which are not responsive to the cancer treatment, wherein a gene expression profile for the genes or subset of genes in the first biological sample similar to the gene expression profile for the genes or subset of genes in biological samples from lymphoma patients which are responsive to the cancer treatment indicates that the first lymphoma patient will be responsive to the cancer treatment, and a gene expression profile for the genes or subset of genes in first biological sample similar to the gene expression profile for the genes or subset of genes in biological samples from lymphoma patients which are not responsive to the cancer treatment indicates that the first lymphoma patient will not be responsive to the cancer treatment.

In another aspect, provided herein are methods for predicting the responsiveness of a lymphoma patient to a cancer treatment comprising: (a) determining the expression level of at least one gene of Table 1 in a biological sample from a lymphoma patient; (b) comparing the expression level of the at least one gene of step (a) with the expression level of the at least one gene in a reference biological sample from a reference lymphoma patient, wherein the reference lymphoma patient is responsive to the cancer treatment, and wherein if the expression level of the at least one gene in the biological sample is similar to the expression level of the at least one gene in the reference biological sample indicates that the lymphoma patient is not likely to be responsive to the cancer treatment. In certain embodiments, the method further comprises obtaining the biological sample from the lymphoma patient. In certain embodiments, the at least one gene comprises five or more genes of Table 1.

In another aspect, provided herein are methods for predicting the responsiveness of a lymphoma patient to a cancer treatment comprising: (a) determining the expression level of at least one gene of Table 1 in a biological sample from a lymphoma patient; (b) comparing the expression level of the at least one gene of step (a) with the expression level of the at least one gene in reference biological samples from a group of reference lymphoma patients, wherein the reference lymphoma patients are responsive to the cancer treatment, and wherein if the expression level of the at least one gene in the biological sample is similar to the expression level of the at least one gene in the reference biological samples indicates that the lymphoma patient is not likely to be responsive to the cancer treatment. In certain embodiments, the method further comprises obtaining the biological sample from the lymphoma patient. In certain embodiments, the at least one gene comprises five or more genes of Table 1. In certain embodiments, the expression level of the at least one gene in reference biological samples is a mean or median value of the expression levels of the gene measured in the reference biological samples of the reference lymphoma patients.

In another aspect, provided herein are methods for predicting the responsiveness of a lymphoma patient to a cancer treatment comprising: (a) determining the expression level of at least one gene of Table 1 in a biological sample of a lymphoma patient; and (b) comparing the expression level of the at least one gene in the biological sample to: (i) the expression level of the at least one gene in biological samples from lymphoma patients who are responsive to the cancer treatment, and (ii) the expression level of the at least one gene in biological samples from lymphoma patients who are not responsive to the cancer treatment, wherein if the expression level of (a) is similar to the expression level of (i), it indicates that the lymphoma patient is likely to be responsive to the cancer treatment; and if the expression level of (a) is similar to the expression level of (ii), it indicates that the lymphoma patient is not likely to be responsive to the cancer treatment. In certain embodiments, the method further comprises obtaining the biological sample from the lymphoma patient. In certain embodiments, the at least one gene comprises five or more genes of Table 1. In certain embodiments, the expression level of the at least one gene in biological samples from lymphoma patients who are not responsive or responsive to the cancer treatment is a mean expression level or a median expression level of the expression levels of the at least one gene measured in biological samples from lymphoma patients who are not responsive or responsive to the cancer treatment.

In certain embodiments, two expression levels of a gene are similar means that one expression level of the gene is within at most 10% (e.g., 1%, 2%, 3%, 4%, 5%, 6%, 7%, 8%, 9%, or 10%) of the other expression level of the same gene. In certain embodiments, the two expression levels are measured in an absolute scale. In certain embodiments, one expression level is the expression level of a gene in a biological sample from a lymphoma patient, and the other expression level is the expression level of the same gene in a reference biological sample from a reference lymphoma patient. In certain embodiments, one expression level is the expression level of a gene in a biological sample from a first lymphoma patient, and the other expression level is the expression level of the same gene in a biological sample from a second lymphoma patient.

In some embodiments, two expression levels are similar means that one expression level of one gene in a biological sample is within one standard deviation or standard error of the mean value of the expression levels the same gene in biological samples from a group of subjects (e.g., a group of reference lymphoma patients, a group of lymphoma patients who are responsive to the cancer treatment, or a group of lymphoma patients who are not responsive to the cancer treatment). In some embodiments, two expression levels of two groups of subjects are similar means that the mean value of the expression levels of one gene in biological samples of one group of subjects is within one standard deviation or standard error of the mean value of the expression levels of the same gene in biological samples of another group of subjects. In some embodiments, two expression levels of two groups of subjects are similar means that a hypothesis test such as a Wilcoxon or t-test failed to reject the null hypothesis of the two means or two medians being equal at a predefined significance level, wherein each mean or median is the mean or median value of the expression levels of the same gene in biological samples of one of the two groups of subjects. In some embodiments, the group of subjects are lymphoma patients who are responsive to the cancer treatment. In some embodiments, the group of subjects are lymphoma patients who are not responsive to the cancer treatment. In some embodiments, the group of subjects are a group of reference lymphoma patients.

In some embodiments the determining step of the methods described herein comprising determining the expression of all genes listed in Table 1. In some embodiments, the expression levels of all genes listed in Table 1 are determined and compared. In some embodiments, the determining step of the methods described herein comprise determining the expression of at least one gene (e.g., one, two, three, four, five or more) selected from or all genes from the group consisting of ABHD10, ACO1, ACTN4, AGRP, AKAP13, ALDH1A1, ALG13, AMT, ANKZF1, AOAH, AP1G2, AP3S1, APRT, ARG1, ARHGDIA, ARHGEF7, ART4, ASH1L, ATIC, ATP6V1G2, ATP9B, BAZ2A, BLNK, BPNT1, BRIP1, BTF3, BUB3, C1QBP, C2, CACUL1, CADPS, CAPZB CARD11, CBX5, CCDC136, CCR2, CCT7, CCT8, CD37, CD46, CDC25A, CDK12, CENPW, CEP85L, CEP97, CFH, CHD2, CHI3L1, CHKA, CIB1, CKAP2, CLCN7, CLEC7A, CLIC1, CLK1, CMSS1, COG7, COL1A2, COL4A3, CORO1A, COX6C, CPD, CRBN, CSF1, CUEDC2, CUX1, CXCL10, DDHD1, DDX58, DIMT1, DNAJA3, DNAJC10, DNMT1, DYNLT1, E2F1, EBAG9, EIF1AX, EIF2B5, EIF3I, EIF5A, ELF1, ELMO1, EMP3, ENO1, EPS15, ERGIC2, ERH, ESD, EYS, FBXO46, FLNA, FOXP1, FPR1, FUBP1, FUS, GALM GAPDH, GATM, GBP5, GIMAP4, GJD3, GLUL, GNA13 GNB2, GNS, GPR82, GPX1, GRIP1, HAMP, HEXIM1, HNMT, HNRNPA2B1, HNRNPU, HSD11B1L, HSP90AA1, HSPD1, HSPE1, IDS, IFI30, IFITM3, IKZF1, IL24, IMPDH2, IST1, ITGB2, JAK1, KIF14, KIF4A, KLHL14, KLHL23, LAP3, LATS1, LMO4, LONP2, LPAR6, LRCH4, LRRC37A2, LRRC37A3, LRRC59, LY9, LYRM1, MAP3K14, MAP4K2, MAPK14, MARS2, MAT2A, MAX, MCL1, MCM4, MGAT4A, MRPL43, MRPS15, MRPS9, MSH6, MVB12A, MVB12B, MVP, MYBL2, MYOF, NACA, NCAPG2, NCBP2, NCL, NFE2L2, NFKBIA, NRXN1, NUDT21, ORC6, P2RX7, PAICS, PARG, PARP9, PAX5, PCNA, PDE9A, PFKL, PGAM1, PIF1, PILRB, PKIA, PKM, PKN2, PLEKHF2, PLK1 PMM2, PNP, POLA1, POLR2E, POM121, PPA1, PPIA, PPP1R9B, PRDM15, PRDX5, PRKCB, PRKCH, PRMT1, PRPF4, RPF6, PRRC2C, PSMC1, PSMD13, PSME2, PTBP3, PTENP1, PTPRC, R3HDM1, RAB32, RAMP3, RANBP2, RBM3, RBM33, RBP1, RFC1, RNASEL, RNF38, RPL30, RPL32P3, RPRD2, RPS3, RPS4X, RRP9, S100A11, S100Z, S1PR2, SAAL1, SBNO1, SCAMP2, SCARB1, SCARB2, SDCBP, SELPLG, SENP7, SERPINA3, SERPINB1, SF1, SF3A1, SFPQ, SFXN3, SH3BGRL3, SH3BP1, SLAMF8, SLC35D1, SMARCA4, SMARCC1, SMG5, SNORA21, SNORA71B, SNORD104, SNRPA, SNRPD1, SNTA1, SNX29, SOBP, SOGA1, SP140, SP3, SPEN, SRGN, SRSF6, STAG3, STK4, STMN1, SUMO2, SYNJ2, SYPL1, SYTL3, TAGLN2, TAP2, TARDBP, TBCA, TGDS, TGOLN2, THRAP3, TIMM10, TLK1, TLN1, TMBIM4, TMEM223, TNFRSF1A, TONSL, TP53INP1, TPM3, TRA2B, TRAM1, TRAPPC12, TRIOBP, TRIP13, TRMT1L, TRNT1, TSPYL2, TSSK4, TUBA1B, TUBA1C, TWIST1, UBE2B, UBE2D2, UBE2G1, UXT, VASH1, VAV1, VDAC1, WEE1, XRCC6, YTHDC1, YWHAE, ZBED5, ZBTB37, ZFAND4, ZFAND5, ZMAT1, ZNF101, ZNF107, ZNF146, ZNF207, ZNF318, ZNF367, ZNF480, and ZWINT.

In some embodiments, the cancer treatment is a combination treatment with rituximab (Rituxan), cyclophosphamide, doxorubicin, vincristine, and prednisone (R-CHOP).

In another aspect, provided herein are methods of treating a lymphoma patient, comprising: (i) identifying a lymphoma patient who is likely to be responsive to the cancer treatment as predicted using the predicting methods described herein above; and (ii) administering to the lymphoma patient the cancer treatment.

In another aspect, provided herein are methods of treating a lymphoma patient, comprising: (i) identifying a lymphoma patient who is not likely to be responsive to the cancer treatment as predicted using the predicting methods described herein above; and (ii) administering to the lymphoma patient an alternative cancer treatment. In some embodiments, the alternative cancer treatment is a BET inhibitor or a CDK inhibitor.

Additionally, in some embodiments, all genes listed in Table 1 can be used as biomarkers to predict the responsiveness of a lymphoma (e.g., DLBCL) patient to a treatment.

In another aspect, the subgroups provided herein (e.g., A1-A7) can be characterized and/or identified based on Bcl6 signature scores as shown in the example section below. Therefore, Bcl6 signature score can also be used as a way to classify a patient into one of the 8 subgroups for the purpose of determining a patient's responsiveness to a treatment.

In another aspect, as shown in the example section below and in Tables 6 and 7, mutational data were collected and interpreted in the context of the identified subgroups. Thus, in some embodiments, the mutation profile of each subgroup (or cluster) or a subset thereof can also be used to identify a subgroup, or to classify a patient into one of the subgroups for the purpose of determining the patient's responsiveness to a treatment.

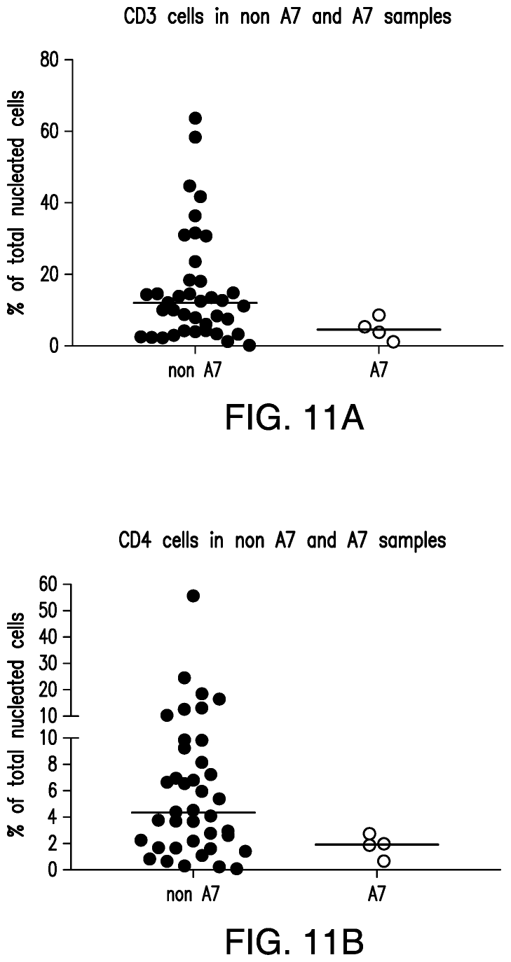

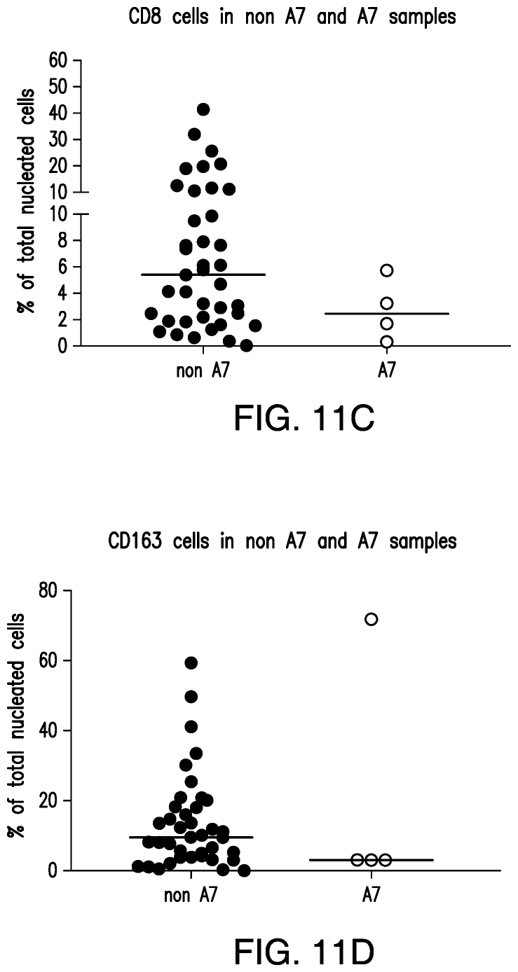

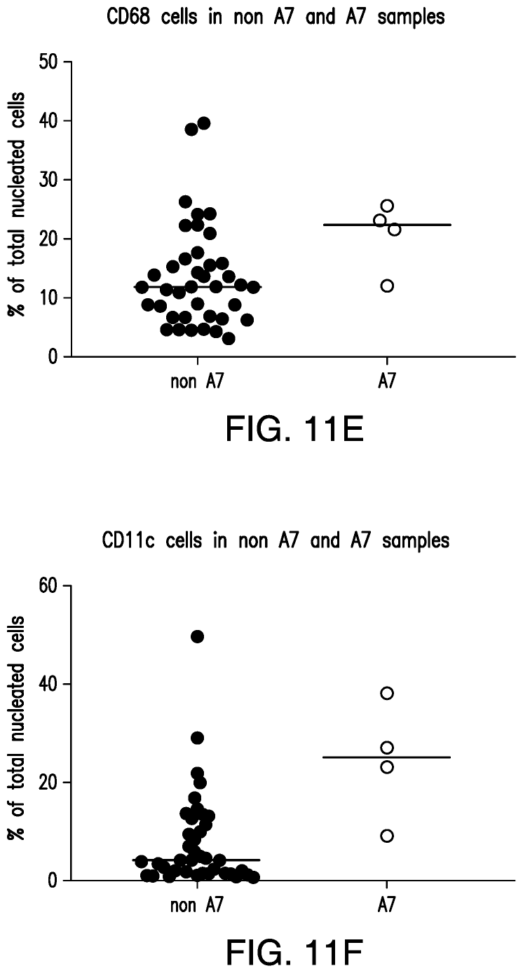

The subgroups (or clusters) provided herein were also characterized based on total counts of different T cell populations (e.g., CD3, CD4, CD8, CD163, CD68, and/or CD11c cells) as shown in the example section below and in FIGS. 11A-11F. Therefore, in yet another aspect, proportions of different T cell populations (e.g., CD3, CD4, CD8, CD163, CD68, and/or CD11c cells) can be used to identify a subgroup or to classify a patient into one of the subgroups for the purpose of determining the patient's responsiveness to a treatment.

### 5.4 Administration Methods

In some embodiments, the methods provided herein comprise administering a first cancer treatment compound to a lymphoma patient predicted to be responsive to the first cancer treatment. Also provided herein are methods of treating patients who have been previously treated for cancer (e.g., DLBCL or a subtype thereof) but are non-responsive to a first cancer treatment (e.g., standard therapies). Also provided herein are methods of treating patients who have not previously been treated. The invention also encompasses methods of treating patients regardless of patient's age, although some diseases or disorders are more common in certain age groups. The invention further encompasses methods of treating patients who have undergone surgery in an attempt to treat the disease or condition, as well as those who have not. Because patients with cancer have heterogeneous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual patient with cancer (e.g., DLBCL or a subtype thereof).

In certain embodiments, a therapeutically or prophylactically effective amount of the cancer treatment is from about 0.005 mg/day to about 1,000 mg/day, from about 0.01 mg/day to about 500 mg per day, from about 0.01 mg/day to about 250 mg/day, from about 0.01 mg/day to about 100 mg/day, from about 0.1 mg/day to about 100 mg/day, from about 0.5 mg/day to about 100 mg/day, from about 1 mg/day to about 100 mg/day, from about 0.01 mg/day to about 50 mg/day, from about 0.1 mg/day to about 50 mg/day, from about 0.5 mg/day to about 50 mg/day, from about 1 mg/day to about 50 mg/day, from about 0.02 mg/day to about 25 mg/day, or from about 0.05 mg/day to about 10 mg/day.

In certain embodiments, the therapeutically or prophylactically effective amount is about 0.1 mg/day, about 0.2 mg/day, about 0.5 mg/day, about 1 mg/day, about 2 mg/day, about 5 mg/day, about 10 mg/day, about 15 mg/day, about 20 mg/day, about 25 mg/day, about 30 mg/day, about 40 mg/day, about 45 mg/day, about 50 mg/day, about 60 mg/day, about 70 mg/day, about 80 mg/day, about 90 mg/day, about 100 mg/day, or about 150 mg/day.

In some embodiments, the recommended daily dose range of the cancer treatment for the conditions described herein lie within the range of from about 0.1 mg/day to about 50 mg/day, preferably given as a single once-a-day dose, or in divided doses throughout a day. In some embodiments, the dosage ranges from about 1 mg/day to about 50 mg/day. In some embodiments, the dosage ranges from about 0.5 mg/day to about 5 mg/day. In some embodiments, the specific doses per day are 0.1 mg/day, 0.2 mg/day, 0.5 mg/day, 1 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, 20 mg/day, 21 mg/day, 22 mg/day, 23 mg/day, 24 mg/day, 25 mg/day, 26 mg/day, 27 mg/day, 28 mg/day, 29 mg/day, 30 mg/day, 31 mg/day, 32 mg/day, 33 mg/day, 34 mg/day, 35 mg/day, 36 mg/day, 37 mg/day, 38 mg/day, 39 mg/day, 40 mg/day, 41 mg/day, 42 mg/day, 43 mg/day, 44 mg/day, 45 mg/day, 46 mg/day, 47 mg/day, 48 mg/day, 49 mg/day, or 50 mg/day.

In some embodiments, the recommended starting dosage may be 0.5 mg/day, 1 mg/day, 2 mg/day, 3 mg/day, 4 mg/day, 5 mg/day, 10 mg/day, 15 mg/day, 20 mg/day, 25 mg/day, or 50 mg/day. In some embodiments, the recommended starting dosage may be 0.5 mg/day, 1 mg/day, 2 mg/day, 3 mg/day, 4 mg/day, or 5 mg/day. The dose may be escalated to 10 mg/day, 15 mg/day, 20 mg/day, 25 mg/day, 30 mg/day, 35 mg/day, 40 mg/day, 45 mg/day, or 50 mg/day.

In some embodiments, the therapeutically or prophylactically effective amount is from about 0.001 mg/kg/day to about 100 mg/kg/day, from about 0.01 mg/kg/day to about 50 mg/kg/day, from about 0.01 mg/kg/day to about 25 mg/kg/day, from about 0.01 mg/kg/day to about 10 mg/kg/day, from about 0.01 mg/kg/day to about 9 mg/kg/day, from about 0.01 mg/kg/day to about 8 mg/kg/day, from about 0.01 mg/kg/day to about 7 mg/kg/day, from about 0.01 mg/kg/day to about 6 mg/kg/day, from about 0.01 mg/kg/day to about 5 mg/kg/day, from about 0.01 mg/kg/day to about 4 mg/kg/day, from about 0.01 mg/kg/day to about 3 mg/kg/day, from about 0.01 mg/kg/day to about 2 mg/kg/day, or from about 0.01 mg/kg/day to about 1 mg/kg/day.

In some embodiments, the administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, www.fda.gov). For example, a dose of 1 mg/kg/day for a 65 kg human is approximately equal to 38 mg/m²/day.

In certain embodiments, the amount of the cancer treatment administered is sufficient to provide a plasma concentration of the compound at steady state, ranging from about 0.001 M to about 500 μM, from about 0.002 μM to about 200 μM, from about 0.005 μM to about 100 μM, from about 0.01 μM to about 50 μM, from about 1 μM to about 50 μM, from about 0.02 μM to about 25 μM, from about 0.05 μM to about 20 μM, from about 0.1 M to about 20 μM, from about 0.5 μM to about 20 μM, or from about 1 μM to about 20 μM.

In some embodiments, the amount of the cancer treatment administered is sufficient to provide a plasma concentration of the compound at steady state, ranging from about 5 nM to about 100 nM, from about 5 nM to about 50 nM, from about 10 nM to about 100 nM, from about 10 nM to about 50 nM, or from about 50 nM to about 100 nM.

As used herein, the term “plasma concentration at steady state” is the concentration reached after a period of administration of a cancer treatment provided herein. Once steady state is reached, there are minor peaks and troughs on the time-dependent curve of the plasma concentration of the cancer treatment.

In some embodiments, the amount of the cancer treatment administered is sufficient to provide a maximum plasma concentration (peak concentration) of the compound, ranging from about 0.001 μM to about 500 μM, from about 0.002 μM to about 200 μM, from about 0.005 μM to about 100 μM, from about 0.01 μM to about 50 μM, from about 1 μM to about 50 μM, from about 0.02 μM to about 25 μM, from about 0.05 μM to about 20 μM, from about 0.1 μM to about 20 μM, from about 0.5 μM to about 20 μM, or from about 1 μM to about 20 μM.

In some embodiments, the amount of the cancer treatment administered is sufficient to provide a minimum plasma concentration (trough concentration) of the compound, ranging from about 0.001 μM to about 500 μM, from about 0.002 μM to about 200 μM, from about 0.005 μM to about 100 μM, from about 0.01 μM to about 50 μM, from about 1 μM to about 50 μM, from about 0.01 μM to about 25 μM, from about 0.01 μM to about 20 μM, from about 0.02 μM to about 20 μM, from about 0.02 μM to about 20 μM, or from about 0.01 μM to about 20 μM.

In some embodiments, the amount of the cancer treatment administered is sufficient to provide an area under the curve (AUC) of the compound, ranging from about 100 ng*hr/mL to about 100,000 ng*hr/mL, from about 1,000 ng*hr/mL to about 50,000 ng*hr/mL, from about 5,000 ng*hr/mL to about 25,000 ng*hr/mL, or from about 5,000 ng*hr/mL to about 10,000 ng*hr/mL.

In some embodiments, the lymphoma patient to be treated with one of the methods provided herein has not been treated with anticancer therapy prior to the administration of a standard therapy (e.g., R-CHOP). In some embodiments, the lymphoma patient to be treated with one of the methods provided herein has been treated with anticancer therapy (standard therapies, e.g., R-CHOP) prior to the administration of the second treatment. In some embodiments, the lymphoma patient to be treated with one of the methods provided herein has developed drug resistance to the first cancer treatment.

Depending on the subtype of lymphoma (e.g., DLBCL) to be treated and the subject's condition, the cancer treatment is administered by parenteral (e.g., intramuscular, intraperitoneal, intravenous, CIV, intracisternal injection or infusion, subcutaneous injection, or implant), inhalation, nasal, vaginal, rectal, sublingual, or topical (e.g., transdermal or local) routes of administration. In some embodiments, the cancer treatment is formulated, alone or together, in suitable dosage unit with pharmaceutically acceptable excipients, carriers, adjuvants, and vehicles, appropriate for each route of administration.

In some embodiments, the cancer treatment is administered parenterally. In some embodiments, the cancer treatment is administered intravenously.

Depending on the state of the lymphoma to be treated and the subject's condition, in some embodiments, the treatment compound is administered by oral, parenteral (e.g., intramuscular, intraperitoneal, intravenous, CIV, intracisternal injection or infusion, subcutaneous injection, or implant), inhalation, nasal, vaginal, rectal, sublingual, or topical (e.g., transdermal or local) routes of administration. In some embodiments, the treatment compound is formulated, alone or together, in suitable dosage unit with pharmaceutically acceptable excipients, carriers, adjuvants and vehicles, appropriate for each route of administration.

In some embodiments, the treatment compound is administered orally. In some embodiments, the treatment compound is administered parenterally. In some embodiments, the treatment compound is administered intravenously.

In some embodiments, the treatment compound can be delivered as a single dose such as, e.g., a single bolus injection, or oral capsules, tablets or pills; or over time, such as, e.g., continuous infusion over time or divided bolus doses over time. In some embodiments, the cancer treatment as described herein can be administered repeatedly if necessary, for example, until the patient experiences stable disease or regression, or until the patient experiences disease progression or unacceptable toxicity.

In some embodiments, the treatment compound can be administered once daily (QD), or divided into multiple daily doses such as twice daily (BID), three times daily (TID), and four times daily (QID). In some embodiments, the administration can be continuous (i.e., daily for consecutive days or every day), intermittent, e.g., in cycles (i.e., including days, weeks, or months of rest without drug). As used herein, the term “daily” is intended to mean that a therapeutic compound is administered once or more than once each day, for example, for a period of time. The term “continuous” is intended to mean that a therapeutic compound is administered daily for an uninterrupted period of at least 7 days to 52 weeks. The term “intermittent” or “intermittently” as used herein is intended to mean stopping and starting at either regular or irregular intervals. In some embodiments, intermittent administration is administration for one to six days per week, administration in cycles (e.g., daily administration for two to eight consecutive weeks, then a rest period with no administration for up to one week), or administration on alternate days. The term “cycling” as used herein is intended to mean that a therapeutic compound is administered daily or continuously but with a rest period.

In some embodiments, the frequency of administration is in the range of about a daily dose to about a monthly dose.

In some embodiments, the cancer treatment can be delivered as a single dose (e.g., a single bolus injection), or over time (e.g., continuous infusion over time or divided bolus doses over time). In some embodiments, the compound can be administered repeatedly if necessary, for example, until the patient experiences stable disease or regression, or until the patient experiences disease progression or unacceptable toxicity. For example, stable disease for solid cancers generally means that the perpendicular diameter of measurable lesions has not increased by 25% or more from the last measurement. Therasse et al., J. Natl. Cancer Inst., 2000, 92(3):205-216. Stable disease or lack thereof is determined by methods known in the art such as evaluation of patient symptoms, physical examination, and visualization of the tumor that has been imaged using X-ray, CAT, PET, MRI scan, or other commonly accepted evaluation modalities.

In some embodiments, the cancer treatment can be administered once daily (QD) or divided into multiple daily doses such as twice daily (BID), three times daily (TID), and four times daily (QID). In some embodiments, the administration can be continuous (i.e., daily for consecutive days or every day) or intermittent, e.g., in cycles (i.e., including days, weeks, or months of rest without drug). As used herein, the term “daily” is intended to mean that a cancer treatment is administered once or more than once each day, for example, for a period of time. The term “continuous” is intended to mean that the cancer treatment is administered daily for an uninterrupted period of at least 10 days to 52 weeks. The term “intermittent” or “intermittently” as used herein is intended to mean stopping and starting at either regular or irregular intervals. For example, intermittent administration of the cancer treatment is administration for one to six days per week, administration in cycles (e.g., daily administration for two to eight consecutive weeks, then a rest period with no administration for up to one week), or administration on alternate days. The term “cycling” as used herein is intended to mean that the cancer treatment is administered daily or continuously but with a rest period. In some embodiments, the rest period is the same length as the treatment period. In some embodiments, the rest period has different length from the treatment period. In some embodiments, the length of cycling is 1, 2, 3, 4, 5, 6, 7, 8, 9, or 10 weeks. In some embodiments of cycling, the cancer treatment is administered daily for a period of 1, 2, 3, 4, 5, 6, 7, 8, 9, 10, 15, 20, 25, or 30 days, followed by a rest period. In some embodiments, the cancer treatment is administered daily for a period of 5 days of a 4-week cycle. In another particular embodiment, the cancer treatment is administered daily for a period of 10 days of a 4-week cycle.

In some embodiments, the frequency of administration is in the range of about a daily dose to about a monthly dose. In some embodiments, administration is once a day, twice a day, three times a day, four times a day, once every other day, twice a week, once every week, once every two weeks, once every three weeks, or once every four weeks. In some embodiments, the cancer treatment is administered once a day. In some embodiments, the cancer treatment is administered twice a day. In some embodiments, the cancer treatment is administered three times a day. In some embodiments, the cancer treatment is administered four times a day.

In some embodiments, the cancer treatment is administered once per day from one day to six months, from one week to three months, from one week to four weeks, from one week to three weeks, or from one week to two weeks. In some embodiments, the cancer treatment is administered once per day for one week, two weeks, three weeks, or four weeks. In some embodiments, the cancer treatment is administered once per day for one week. In some embodiments, the cancer treatment is administered once per day for two weeks. In some embodiments, the cancer treatment is administered once per day for three weeks. In still another embodiment, the cancer treatment is administered once per day for four weeks.

### 5.5 Combination Therapy

One or more additional therapies, such as additional active ingredients or agents, that can be used in combination with the administration of a cancer treatment described herein to treat a lymphoma patient (e.g., a patient having DLBCL). In some embodiments, the one or more additional therapies can be administered prior to, concurrently with, or subsequent to the administration of the compound described herein. Administration of a cancer treatment described herein and an additional active agent (“second active agents”) to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the condition of lymphoma (e.g., DLBCL) being treated. Routes of administration for the additional active agents or ingredients are known to those of ordinary skill in the art. See, e.g., Physicians' Desk Reference.

In some embodiments, the cancer treatment described herein and an additional active agent are cyclically administered to a patient with lymphoma (e.g., DLBCL). Cycling therapy involves the administration of an active agent for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment.

In some embodiments, one or more second active ingredients or agents can be used in the methods and compositions provided herein. Second active agents can be large molecules (e.g., proteins) or small molecules (e.g., synthetic inorganic, organometallic, or organic molecules). Various agents can be used, such as those described in U.S. patent application Ser. No. 16/390,815 or U.S. Provisional Application entitled, “SUBSTITUTED 4-AMINOISOINDOLINE-1,3-DIONE COMPOUNDS AND SECOND ACTIVE AGENTS FOR COMBINED USE,” filed on even date herewith (Attorney Docket No. 14247-390-888), each of which is incorporated herein by reference in their entirety. In some embodiments, exemplary second active agents include, but are not limited to, an HDAC inhibitor (e.g., panobinostat, romidepsin, or vorinostat), a BCL2 inhibitor (e.g., venetoclax), a BTK inhibitor (e.g., ibrutinib or acalabrutinib), an mTOR inhibitor (e.g., everolimus), a PI3K inhibitor (e.g., idelalisib), a PKCβ inhibitor (e.g., enzastaurin), a SYK inhibitor (e.g., fostamatinib), a JAK2 inhibitor (e.g., fedratinib, pacritinib, ruxolitinib, baricitinib, gandotinib, lestaurtinib, or momelotinib), an Aurora A kinase inhibitor (e.g., alisertib), an EZH2 inhibitor (e.g., tazemetostat, GSK126, CPI-1205, 3-deazaneplanocin A, EPZ005687, EI1, UNC1999, or sinefungin), a BET inhibitor (e.g., birabresib or 4-[2-(cyclopropylmethoxy)-5-(methanesulfonyl)phenyl]-2-methylisoquinolin-1(2H)-one), a hypomethylating agent (e.g., 5-azacytidine or decitabine), a chemotherapy (e.g., bendamustine, doxorubicin, etoposide, methotrexate, cytarabine, vincristine, ifosfamide, or melphalan), or an epigenetic compound (e.g., a DOT1L inhibitor such as pinometostat, a HAT inhibitor such as C646, a WDR5 inhibitor such as OICR-9429, a HDAC6 inhibitor such as ACY-241, a DNMT1 selective inhibitor such as GSK3484862, a LSD-1 inhibitor such as Compound C or seclidemstat, a G9A inhibitor such as UNC 0631, a PRMT5 inhibitor such as GSK3326595, a BRPF1B/2 inhibitor such as OF-1, a BRD9/7 inhibitor such as LP99, a SUV420H1/H2 inhibitor such as A-196, a Menin-MLL inhibitor such as MI-503, a CARM1 inhibitor such as EZM2302, a BRD9 such as an inhibitor dBrd9), aiolos/ikaros degrading cereblon E3 ligase modulator (CELMoDs), CREBBp2 inhibitors, anti-CD79b antibody, CD19 CAR-T, inhibitors of p53 (nutlins), Bc16 inhibitors, CREBBp2 CELMoDs, CD79b CELMoDs, CD19 CELMoDs, p53 (nutlins) CELMoDs, Bc16 CELMoDs, inhibitors of ligand directed degradation (LDD) of CREBBP2, inhibitors of LDD of CD79b, inhibitors of LDD of CD19, inhibitors of LDD of p53 (nutlins), inhibitors of LDD of Bc6, inhibitors of LDD of CK1a, inhibitors of LDD of IRAK4 (e.g., in MYD88 L265p lymphoma), MALT1 inhibitors such as JNJ-67856633, MAT2A inhibitors (e.g., for 9p21 deletions), anti-CD3×anti-CD19 bispecific antibodies, and anti-CD3×anti-CD20 bispecific antibodies.

In some embodiments, the methods further include administration of one or more of rituximab, cyclophosphamide, doxorubicin, vincristine, prednisone, etoposide, Bendamustine (Treanda), lenalidomide, or gemcitabine. In some embodiments, the methods further include administration of one or more of rituximab, cyclophosphamide, doxorubicin, vincristine, prednisone, etoposide, Bendamustine (Treanda), or gemcitabine. In some embodiments, the treatment further includes treatment with one or more of R-CHOP (rituximab plus cyclophosphamide, doxorubicin, vincristine, and prednisone), R EPOCH (etoposide, rituximab plus cyclophosphamide, doxorubicin, vincristine, and prednisone), stem cell transplant, Bendamustine (Treanda) plus rituximab, rituximab, lenalidomide plus rituximab, or gemcitabine-based combinations. In some embodiments, the treatment further includes treatment with one or more of R-CHOP (rituximab plus cyclophosphamide, doxorubicin, vincristine, and prednisone), R EPOCH (etoposide, rituximab plus cyclophosphamide, doxorubicin, vincristine, and prednisone), stem cell transplant, Bendamustine (Treanda) plus rituximab, rituximab, or gemcitabine-based combinations. In certain embodiments, the second active agent is rituximab, as provided in U.S. Provisional Application 62/833,432.

In some embodiments, the second active agent used in the methods provided herein is a histone deacetylase (HDAC) inhibitor. In some embodiments, the HDAC inhibitor is panobinostat, romidepsin, or vorinostat, or a stereoisomer, mixture of stereoisomers, tautomer, isotopolog, or pharmaceutically acceptable salt thereof.

In some embodiments, the second active agent used in the methods provided herein is a B-cell lymphoma 2 (BCL2) inhibitor. In some embodiments, the BCL2 inhibitor is venetoclax, or a tautomer, isotopolog, or pharmaceutically acceptable salt thereof. In some embodiments, the BCL2 inhibitor is venetoclax.

In some embodiments, the second active agent used in the methods provided herein is a Bruton's tyrosine kinase (BTK) inhibitor. In some embodiments, the BTK inhibitor is ibrutinib, or acalabrutinib, or a stereoisomer, mixture of stereoisomers, tautomer, isotopolog, or pharmaceutically acceptable salt thereof. In some embodiments, the BTK inhibitor is ibrutinib, or a stereoisomer, mixture of stereoisomers, tautomer, isotopolog, or pharmaceutically acceptable salt thereof. In some embodiments, the BTK inhibitor is ibrutinib. In some embodiments, the BTK inhibitor is acalabrutinib, or a stereoisomer, mixture of stereoisomers, tautomer, isotopolog, or pharmaceutically acceptable salt thereof. In some embodiments, the BTK inhibitor is acalabrutinib.

In some embodiments, the second active agent used in the methods provided herein is a mammalian target of rapamycin (mTOR) inhibitor. In some embodiments, the mTOR inhibitor is rapamycin or an analog thereof (also termed rapalog). In some embodiments, the mTOR inhibitor is everolimus, or a stereoisomer, mixture of stereoisomers, tautomer, isotopolog, or pharmaceutically acceptable salt thereof. In some embodiments, the mTOR inhibitor is everolimus.

In some embodiments, the second active agent used in the methods provided herein is a phosphoinositide 3-kinase (PI3K) inhibitor. In some embodiments, the PI3K inhibitor is idelalisib, or a stereoisomer, mixture of stereoisomers, tautomer, isotopolog, or pharmaceutically acceptable salt thereof. In some embodiments, the PI3K inhibitor is idelalisib.

In some embodiments, the second active agent used in the methods provided herein is a protein kinase C beta (PKCβ or PKC-β) inhibitor. In some embodiments, the PKCβ inhibitor is enzastaurin, or a stereoisomer, mixture of stereoisomers, tautomer, isotopolog, or pharmaceutically acceptable salt thereof. In some embodiments, the PKCβ inhibitor is enzastaurin. In some embodiments, the PKCβ inhibitor is a pharmaceutically acceptable salt of enzastaurin. In some embodiments, the PKCβ inhibitor is a hydrochloride salt of enzastaurin. In some embodiments, the PKCβ inhibitor is a bis-hydrochloride salt of enzastaurin.

In some embodiments, the second active agent used in the methods provided herein is a spleen tyrosine kinase (SYK) inhibitor. In some embodiments, the SYK inhibitor is fostamatinib, or a tautomer, isotopolog, or pharmaceutically acceptable salt thereof. In some embodiments, the SYK inhibitor is fostamatinib. In some embodiments, the SYK inhibitor is a pharmaceutically acceptable salt of fostamatinib. In some embodiments, the SYK inhibitor is fostamatinib disodium hexahydrate.

In some embodiments, the second active agent used in the methods provided herein is a Janus kinase 2 (JAK2) inhibitor. In some embodiments, the JAK2 inhibitor is fedratinib, pacritinib, ruxolitinib, baricitinib, gandotinib, lestaurtinib, or momelotinib, or a stereoisomer, mixture of stereoisomers, tautomer, isotopolog, or pharmaceutically acceptable salt thereof.

In some embodiments, the JAK2 inhibitor is fedratinib, or a tautomer, isotopolog, or pharmaceutically acceptable salt thereof. In some embodiments, the JAK2 inhibitor is fedratinib.

In some embodiments, the JAK2 inhibitor is pacritinib, or a tautomer, isotopolog, or pharmaceutically acceptable salt thereof. In some embodiments, the JAK2 inhibitor is pacritinib.

In some embodiments, the JAK2 inhibitor is ruxolitinib, or a stereoisomer, mixture of stereoisomers, tautomer, isotopolog, or pharmaceutically acceptable salt thereof. In some embodiments, the JAK2 inhibitor is ruxolitinib. In some embodiments, the JAK2 inhibitor is a pharmaceutically acceptable salt of ruxolitinib. In some embodiments, the JAK2 inhibitor is ruxolitinib phosphate.

In some embodiments, the second active agent used in the methods provided herein is an aurora A kinase inhibitor. In some embodiments, the aurora A kinase inhibitor is alisertib, or a tautomer, isotopolog, or pharmaceutically acceptable salt thereof. In some embodiments, the aurora A kinase inhibitor is alisertib.

In some embodiments, the second active agent used in the methods provided herein is an enhancer of zeste homolog 2 (EZH2) inhibitor. In some embodiments, the EZH2 inhibitor is tazemetostat, GSK126, CPI-1205, 3-deazaneplanocin A (DZNep), EPZ005687, El1, UNC1999, or sinefungin, or a stereoisomer, mixture of stereoisomers, tautomer, isotopolog, or pharmaceutically acceptable salt thereof.

In some embodiments, the EZH2 inhibitor is tazemetostat, or a tautomer, isotopolog, or pharmaceutically acceptable salt thereof. In some embodiments, the EZH2 inhibitor is tazemetostat.

In some embodiments, the EZH2 inhibitor is GSK126, or a stereoisomer, mixture of stereoisomers, tautomer, isotopolog, or pharmaceutically acceptable salt thereof. In some embodiments, the EZH2 inhibitor is GSK126 (also known as GSK-2816126).

In some embodiments, the EZH2 inhibitor is CPI-1205, or a stereoisomer, mixture of stereoisomers, tautomer, isotopolog, or pharmaceutically acceptable salt thereof. In some embodiments, the EZH2 inhibitor is CPI-1205.

In some embodiments, the EZH2 inhibitor is 3-deazaneplanocin A. In some embodiments, the EZH2 inhibitor is EPZ005687. In some embodiments, the EZH2 inhibitor is El1. In some embodiments, the EZH2 inhibitor is UNC1999. In some embodiments, the EZH2 inhibitor is sinefungin.

In some embodiments, the second active agent used in the methods provided herein is a hypomethylating agent. In some embodiments, the hypomethylating agent is 5-azacytidine or decitabine, or a stereoisomer, mixture of stereoisomers, tautomer, isotopolog, or pharmaceutically acceptable salt thereof.

In some embodiments, the hypomethylating agent is 5-azacytidine, or a stereoisomer, mixture of stereoisomers, tautomer, isotopolog, or pharmaceutically acceptable salt thereof. In some embodiments, the hypomethylating agent is 5-azacytidine.

In some embodiments, the hypomethylating agent is decitabine, or a stereoisomer, mixture of stereoisomers, tautomer, isotopolog, or pharmaceutically acceptable salt thereof. In some embodiments, the hypomethylating agent is decitabine.

In some embodiments, the second active agent used in the methods provided herein is a chemotherapy. In some embodiments, the chemotherapy is bendamustine, doxorubicin, etoposide, methotrexate, cytarabine, vincristine, ifosfamide, or melphalan, or a stereoisomer, mixture of stereoisomers, tautomer, isotopolog, prodrug, or pharmaceutically acceptable salt thereof.

In certain embodiments, the second therapeutic agent is administered before, after or simultaneously with a cancer treatment described herein. Administration of a cancer treatment described herein and a second therapeutic agent to a patient can occur simultaneously or sequentially by the same or different routes of administration. The suitability of a particular route of administration employed for a particular second drug or agent will depend on the second therapeutic agent itself (e.g., whether it can be administered orally or topically without decomposition prior to entering the blood stream) and the subject being treated. Particular routes of administration for the second drug or agents or ingredients are known to those of ordinary skill in the art. See, e.g., The Merck Manual, 448 (17th ed., 1999).

Any combination of the above therapeutic agents, suitable for treatment of the diseases or symptoms thereof, can be administered. Such therapeutic agents can be administered in any combination at the same time or as a separate course of treatment.

As used herein, the term “in combination” does not restrict the order in which therapies (e.g., prophylactic and/or therapeutic agents) are administered to a patient with a disease or disorder. Administration of a second active agent provided herein, to a patient can occur simultaneously or sequentially by the same or different routes of administration. The suitability of a particular route of administration employed for a particular active agent will depend on the active agent itself (e.g., whether it can be administered orally without decomposing prior to entering the blood stream).

### 5.6 Pharmaceutical Compositions

In some embodiments, the cancer treatment provides herein and/or the additional active agent provided herein are formulated in a pharmaceutical composition, and the method provide herein comprises administering to a lymphoma (e.g., DLBCL) patient a pharmaceutical composition comprising the cancer treatment.

In some embodiments, the pharmaceutical compositions provided herein comprise therapeutically effective amounts of one or more of the cancer treatment provided herein and a pharmaceutically acceptable carrier, diluents, or excipient. In some embodiments, the compounds are formulated as the sole pharmaceutically active ingredient in the composition or are combined with other active ingredients.

In some embodiments, the cancer treatment provided herein can be formulated into suitable pharmaceutical compositions for different routes of administration, such as injection, sublingual and buccal, rectal, vaginal, ocular, otic, nasal, inhalation, nebulization, cutaneous, or transdermal. Typically, the compounds described above are formulated into pharmaceutical compositions using techniques and procedures well known in the art (see, e.g., Ansel, Introduction to Pharmaceutical Dosage Forms, (7th ed. 1999)).

In some embodiments, the compositions comprise effective concentrations of one or more compounds or pharmaceutically acceptable salts are mixed with a suitable pharmaceutical carrier or vehicle. In some embodiments, the concentrations of the compounds in the compositions are effective for delivery of an amount, upon administration, that treats, prevents, or ameliorates one or more of the symptoms and/or progression of lymphoma (e.g., DLBCL).

In some embodiments, the active compound is in an amount sufficient to exert a therapeutically useful effect in the absence of undesirable side effects on the patient treated. The therapeutically effective concentration is determined empirically by testing the compounds in in vitro and in vivo systems described herein and then extrapolated therefrom for dosages for humans. The concentration of active compound in the pharmaceutical composition depends on absorption, tissue distribution, inactivation, and excretion rates of the active compound, the physicochemical characteristics of the compound, the dosage schedule, and amount administered as well as other factors known to those of skill in the art.

In some embodiments, the pharmaceutically therapeutically active compounds and salts thereof are formulated and administered in unit dosage forms or multiple dosage forms. Unit dose forms as used herein refer to physically discrete units suitable for human and animal subjects and packaged individually as is known in the art. Each unit dose comprises a predetermined quantity of the therapeutically active compound sufficient to produce the desired therapeutic effect, in association with the required pharmaceutical carriers, vehicles, or diluents. Examples of unit dose forms include ampoules and syringes and individually packaged tablets or capsules. Unit dose forms are administered in fractions or multiples thereof. A multiple dose form is a plurality of identical unit dosage forms packaged in a single container to be administered in segregated unit dose form. Examples of multiple dose forms include vials, bottles of tablets or capsules, or bottles of pints or gallons. Hence, multiple dose form is a multiple of unit doses which are not segregated in packaging.

In some embodiments, the precise dosage and duration of treatment are a function of the disease being treated and are determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens are adjusted over time according to the individual need and the professional judgment of the person administering or supervising the administration of the compositions, and that the concentration ranges set forth herein are exemplary only and are not intended to limit the scope or practice of the claimed compositions.

In some embodiments, solutions or suspensions used for parenteral, intradermal, subcutaneous, or topical application can include any of the following components: a sterile diluents (such as water, saline solution, fixed oil, polyethylene glycol, glycerine, propylene glycol, dimethyl acetamide, or other synthetic solvent), antimicrobial agents (such as benzyl alcohol and methyl parabens), antioxidants (such as ascorbic acid and sodium bisulfate), chelating agents (such as ethylenediaminetetraacetic acid (EDTA)), buffers (such as acetates, citrates, and phosphates), and agents for the adjustment of tonicity (such as sodium chloride or dextrose). Parenteral preparations can be enclosed in ampoules, pens, disposable syringes, or single or multiple dose vials made of glass, plastic, or other suitable material.

In some embodiments, sustained-release preparations can also be prepared. Suitable examples of sustained-release preparations include semipermeable matrices of solid hydrophobic polymers comprising the compound provided herein, which matrices are in the form of shaped articles, e.g., films or microcapsule. Examples of sustained-release matrices include iontophoresis patches, polyesters, hydrogels (for example, poly(2-hydroxyethyl-methacrylate) or poly(vinylalcohol)), polylactides, copolymers of L-glutamic acid and ethyl-L-glutamate, non-degradable ethylene-vinyl acetate, degradable lactic acid-glycolic acid copolymers such as LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular di-sulfate bond formation through thio-disulfide interchange, stabilization may be achieved by modifying sulfhydryl residues, lyophilizing from acidic solutions, controlling moisture content, using appropriate additives, and developing specific polymer matrix compositions.

In some embodiments, further encompassed are anhydrous pharmaceutical compositions and dosage forms comprising a compound provided herein. Anhydrous pharmaceutical compositions and dosage forms provided herein can be prepared using anhydrous or low moisture comprising ingredients and low moisture or low humidity conditions, as known by those skilled in the art. An anhydrous pharmaceutical composition can be prepared and stored such that its anhydrous nature is maintained. Accordingly, anhydrous compositions are packaged using materials known to prevent exposure to water such that they can be included in suitable formulatory kits. Examples of suitable packaging include, but are not limited to, hermetically sealed foils, plastics, unit dose containers (e.g., vials), blister packs, and strip packs.

In some embodiments, dosage forms or compositions comprising active ingredient in the range of from 0.001% to 100% with the balance made up from non-toxic carrier may be prepared. In some embodiments, the compositions comprise from about 0.005% to about 95% active ingredient. In some embodiments, the compositions comprise from about 0.01% to about 90% active ingredient. In some embodiments, the compositions comprise from about 0.1% to about 85% active ingredient. In some embodiments, the compositions comprise from about 0.1% to about 95% active ingredient.

In some embodiments, pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents, and other pharmaceutically acceptable substances.

In some embodiments, examples of aqueous vehicles include sodium chloride injection, Ringer's injection, isotonic dextrose injection, sterile water injection, dextrose and lactated Ringer's injection. Nonaqueous parenteral vehicles include fixed oils of vegetable origin, such as cottonseed oil, corn oil, sesame oil, and peanut oil. Antimicrobial agents in bacteriostatic or fungistatic concentrations must be added to parenteral preparations packaged in multiple dose containers, which include phenols or cresols, mercurials, benzyl alcohol, chlorobutanol, methyl and propyl-p-hydroxybenzoic acid esters, thimerosal, benzalkonium chloride, and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles, and sodium hydroxide, hydrochloric acid, citric acid, or lactic acid for pH adjustment.

In some embodiments, injectables are designed for local and systemic administration. Typically, a therapeutically effective dosage is formulated to comprise a concentration of at least about 0.1% w/w up to about 90% w/w or more, such as more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

Of interest herein are also lyophilized powders, which can be reconstituted for administration as solutions, emulsions, and other mixtures. They may also be reconstituted and formulated as solids or gels.

In some embodiments, the sterile, lyophilized powder is prepared by dissolving a compound provided herein, or a pharmaceutically acceptable salt thereof, in a suitable solvent. In some embodiments, the solvent comprises an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose, or other suitable agent. In some embodiments, the solvent comprises a buffer, such as citrate, phosphate, or other buffers known to those of skill in the art. Subsequent sterile filtration of the solution followed by lyophilization under standard conditions known to those of skill in the art provides the desired formulation. Generally, the resulting solution is apportioned into vials for lyophilization. Each vial comprises a single dosage or multiple dosages of the compound. The lyophilized powder can be stored under appropriate conditions, such as at about 4° C. to room temperature.

In some embodiments, the lyophilized formulations are suitable for reconstitution with a suitable diluent to the appropriate concentration prior to administration. In some embodiments, the lyophilized formulation is stable at room temperature. In some embodiments, the lyophilized formulation is stable at room temperature for up to about 24 months. In some embodiments, the lyophilized formulation is stable at room temperature for up to about 24 months, up to about 18 months, up to about 12 months, up to about 6 months, up to about 3 months or up to about 1 month. In some embodiments, the lyophilized formulation is stable upon storage under accelerated condition of 40° C./75% RH for up to about 12 months, up to about 6 months or up to about 3 months.

In some embodiments, the lyophilized formulation is suitable for reconstitution with an aqueous solution for intravenous administrations. In certain embodiments, the lyophilized formulation provided herein is suitable for reconstitution with water. In some embodiments, the reconstituted aqueous solution is stable at room temperature for up to about 24 hours upon reconstitution. In some embodiments, the reconstituted aqueous solution is stable at room temperature from about 1-24, 2-20, 2-15, 2-10 hours upon reconstitution. In some embodiments, the reconstituted aqueous solution is stable at room temperature for up to about 20, 15, 12, 10, 8, 6, 4 or 2 hours upon reconstitution. In certain embodiments, the lyophilized formulations upon reconstitution have a pH of about 4 to 5.

Active ingredients provided herein can be administered by controlled release means or by delivery devices that are well known to those of ordinary skill in the art. Examples include, but are not limited to, those described in U.S. Pat. Nos. 3,845,770, 3,916,899, 3,536,809, 3,598,123, 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461, 6,419,961, 6,589,548, 6,613,358, 6,699,500, and 6,740,634, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof, to provide the desired release profile in varying proportions. Suitable controlled-release formulations known to those of ordinary skill in the art, including those described herein, can be readily selected for use with the active ingredients provided herein.

### 5.7 Biological Samples

In certain embodiments, the various methods provided herein use samples (e.g., biological samples) from lymphoma (e.g., DLBCL) patients. The patient can be male or female, and can be an adult, child or infant. Samples can be analyzed at a time during an active phase of lymphoma (e.g., DLBCL), or when lymphoma (e.g., DLBCL) is inactive. In some embodiments, a sample is obtained from a patient prior, concurrently with and/or subsequent to administration of a treatment described herein. In some embodiments, a sample is obtained from a patient prior to administration of a treatment described herein. In certain embodiments, more than one sample from a patient can be obtained.

In certain embodiments, the sample comprises body fluids from a subject. Non-limiting examples of body fluids include blood (e.g., peripheral whole blood, peripheral blood), blood plasma, amniotic fluid, aqueous humor, bile, cerumen, cowper's fluid, pre-ejaculatory fluid, chyle, chyme, female ejaculate, interstitial fluid, lymph, menses, breast milk, mucus, pleural fluid, pus, saliva, sebum, semen, serum, sweat, tears, urine, vaginal lubrication, vomit, water, feces, internal body fluids, including cerebrospinal fluid surrounding the brain and the spinal cord, synovial fluid surrounding bone joints, intracellular fluid is the fluid inside cells, and vitreous humour the fluids in the eyeball. In some embodiments, the sample is a blood sample. The blood sample can be obtained using conventional techniques as described in, e.g. Innis et al, editors, PCR Protocols (Academic Press, 1990). White blood cells can be separated from blood samples using convention techniques or commercially available kits, e.g. RosetteSep kit (Stein Cell Technologies, Vancouver, Canada). Sub-populations of white blood cells, e.g. mononuclear cells, B cells, T cells, monocytes, granulocytes or lymphocytes, can be further isolated using conventional techniques, e.g. magnetically activated cell sorting (MACS) (Miltenyi Biotec, Auburn, California) or fluorescently activated cell sorting (FACS) (Becton Dickinson, San Jose, California).

In some embodiments, the blood sample is from about 0.1 mL to about 10.0 mL, from about 0.2 mL to about 7 mL, from about 0.3 mL to about 5 mL, from about 0.4 mL to about 3.5 mL, or from about 0.5 mL to about 3 mL. In some embodiments, the blood sample is about 0.3 mL, 0.4 mL, 0.5 mL, 0.6 mL, 0.7 mL, 0.8 mL, 0.9 mL, 1.0 mL, 1.5 mL, 2.0 mL, 2.5 mL, 3.0 mL, 3.5 mL, 4.0 mL, 4.5 mL, 5.0 mL, 6.0 mL, 7.0 mL, 8.0 mL, 9.0 mL or 10.0 mL.

In some embodiments, the sample used in the present methods comprises a biopsy (e.g., a tumor biopsy). The biopsy can be from any organ or tissue, for example, skin, liver, lung, heart, colon, kidney, bone marrow, teeth, lymph node, hair, spleen, brain, breast, or other organs. In some embodiments, the sample used in the methods described herein comprises a tumor biopsy. Any biopsy technique known by those skilled in the art can be used for isolating a sample from a subject, for instance, open biopsy, close biopsy, core biopsy, incisional biopsy, excisional biopsy, or fine needle aspiration biopsy.

In some embodiments, the sample used in the methods provided herein is obtained from the subject prior to the patient receiving a treatment for lymphoma (e.g., DLBCL). In some embodiments, the sample is obtained from the patient during the subject receiving a treatment for the lymphoma (e.g., DLBCL). In some embodiments, the sample is obtained from the patient after the patient received a treatment for the lymphoma (e.g., DLBCL). In various embodiments, the treatment comprises administering a compound described herein to the subject.

In certain embodiments, the sample comprises a plurality of cells. Such cells can include any type of cells, e.g., stem cells, blood cells (e.g., peripheral blood mononuclear cells), lymphocytes, B cells, T cells, monocytes, granulocytes, immune cells, or tumor or cancer cells. In some embodiments, the tumor or cancer cells or a tumor tissue comprise a tumor biopsy or a tumor explants. In some embodiments, T cells (T lymphocytes) include, for example, helper T cells (effector T cells or Th cells), cytotoxic T cells (CTLs), memory T cells, and regulatory T cells. In some embodiments, the cells used in the methods provided herein are CD3⁺ T cells, e.g., as detected by flow cytometry. The number of T cells used in the methods can range from a single cell to about 10⁹ cells. In some embodiments, B cells (B lymphocytes) include, for example, plasma B cells, dendritic cells, memory B cells, B1 cells, B2 cells, marginal-zone B cells, and follicular B cells. B cells can express immunoglobulins (antibodies, B cell receptor).

In some embodiments, specific cell populations can be obtained using a combination of commercially available antibodies (e.g., Quest Diagnostic (San Juan Capistrano, Calif.); Dako (Denmark)).

In certain embodiments, the sample used in the methods provided herein is from a diseased tissue from a lymphoma (e.g., DLBCL) patient. In certain embodiments, the number of cells used in the methods provided herein can range from a single cell to about 10⁹ cells. In some embodiments, the number of cells used in the methods provided herein is about 1×10⁴, 5×10⁴, 1×10⁵, 5×10⁵, 1×10⁶, 5×10⁶, 1×10⁷, 5×10⁷, 1×10⁸, or 5×10⁸.

In some embodiments, the number and type of cells collected from a subject can be monitored, for example, by measuring changes in morphology and cell surface markers using standard cell detection techniques such as flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, and/or by measuring changes in gene expression using techniques well known in the art, such as PCR and gene expression profiling. These techniques can be used, too, to identify cells that are positive for one or more particular markers. Fluorescence activated cell sorting (FACS) is a well-known method for separating particles, including cells, based on the fluorescent properties of the particles (Kamarch, Methods Enzymol., 1987, 151:150-165). Laser excitation of fluorescent moieties in the individual particles results in a small electrical charge allowing electromagnetic separation of positive and negative particles from a mixture. In some embodiments, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In certain embodiments, subsets of cells are used in the methods provided herein. Methods to sort and isolate specific populations of cells are well-known in the art and can be based on cell size, morphology, or intracellular or extracellular markers. Such methods include, but are not limited to, flow cytometry, flow sorting, FACS, bead based separation such as magnetic cell sorting, size-based separation (e.g., a sieve, an array of obstacles, or a filter), sorting in a microfluidics device, antibody-based separation, sedimentation, affinity adsorption, affinity extraction, density gradient centrifugation, laser capture microdissection, etc.

### 5.8 Methods for Detecting Expression Levels

In some embodiments, the methods provided herein comprise measuring the expression level of at least one gene listed in Table 1. The expression level of the at least one gene can be determined by any known methods in the art.

In some embodiments, the expression level of the at least one gene is determined by measuring the mRNA levels of these genes. Several methods of detecting or quantitating mRNA levels are known in the art. Exemplary methods include but are not limited to northern blots, ribonuclease protection assays, PCR-based methods, and the like. The mRNA sequence can be used to prepare a probe that is at least partially complementary. The probe can then be used to detect the mRNA sequence in a sample, using any suitable assay, such as PCR-based methods, digital PCR (dPCR), Northern blotting, a dipstick assay, and the like.

In some embodiments, a nucleic acid assay for testing for immunomodulatory activity in a biological sample can be prepared. An assay comprises a solid support and at least one nucleic acid contacting the support, where the nucleic acid corresponds to at least a portion of an mRNA. The assay can also have a means for detecting the altered expression of the mRNA in the sample.

In some embodiments, the assay method can be varied depending on the type of mRNA information desired. Exemplary methods include but are not limited to Northern blots and PCR-based methods (e.g., RT-qPCR). Methods such as RT-qPCR can also accurately quantitate the amount of the mRNA in a sample.

Any suitable assay platform can be used to determine the presence of the mRNA in a sample. For example, an assay may be in the form of a dipstick, a membrane, a chip, a disk, a test strip, a filter, a microsphere, a slide, a multiwell plate, or an optical fiber. In some embodiments, an assay system may have a solid support on which a nucleic acid corresponding to the mRNA is attached. The solid support may comprise, for example, a plastic, silicon, a metal, a resin, glass, a membrane, a particle, a precipitate, a gel, a polymer, a sheet, a sphere, a polysaccharide, a capillary, a film a plate, or a slide. The assay components can be prepared and packaged together as a kit for detecting an mRNA.

In some embodiments, the nucleic acid can be labeled, if desired, to make a population of labeled mRNAs. In general, a sample can be labeled using methods that are well known in the art (e.g., using DNA ligase, terminal transferase, or by labeling the RNA backbone, etc.; see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). In some embodiments, the sample is labeled with fluorescent label. Exemplary fluorescent dyes include but are not limited to xanthene dyes, fluorescein dyes, rhodamine dyes, fluorescein isothiocyanate (FITC), 6 carboxyfluorescein (FAM), 6 carboxy-2′,4′,7′,4,7-hexachlorofluorescein (HEX), 6 carboxy 4′, 5′ dichloro 2′, 7′ dimethoxyfluorescein (JOE or J), N,N,N′,N′ tetramethyl 6 carboxyrhodamine (TAMRA or T), 6 carboxy X rhodamine (ROX or R), 5 carboxyrhodamine 6G (R6G5 or G5), 6 carboxyrhodamine 6G (R6G6 or G6), and rhodamine 110; cyanine dyes, e.g. Cy3, Cy5 and Cy7 dyes; Alexa dyes, e.g. Alexa-fluor-555; coumarin, Diethylaminocoumarin, umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, BODIPY dyes, quinoline dyes, Pyrene, Fluorescein Chlorotriazinyl, R110, Eosin, JOE, R6G, Tetramethylrhodamine, Lissamine, ROX, Napthofluorescein, and the like.

In some embodiments, an mRNA assay method can comprise the steps of 1) obtaining surface-bound subject probes; 2) hybridization of a population of mRNAs to the surface-bound probes under conditions sufficient to provide for specific binding (3) post-hybridization washes to remove nucleic acids not bound in the hybridization; and (4) detection of the hybridized mRNAs. The reagents used in each of these steps and their conditions for use may vary depending on the particular application.

In some embodiments, hybridization can be carried out under suitable hybridization conditions, which may vary in stringency as desired. Typical conditions are sufficient to produce probe/target complexes on a solid surface between complementary binding members, i.e., between surface-bound subject probes and complementary mRNAs in a sample. In certain embodiments, stringent hybridization conditions may be employed.

In some embodiments, hybridization is typically performed under stringent hybridization conditions. Standard hybridization techniques (e.g. under conditions sufficient to provide for specific binding of target mRNAs in the sample to the probes) are described in Kallioniemi et al., Science, 258:818-821 (1992) and WO 93/18186. Several guides to general techniques are available, e.g., Tijssen, Hybridization with Nucleic Acid Probes, Parts I and II (Elsevier, Amsterdam 1993). For descriptions of techniques suitable for in situ hybridizations, see Gall et al. Meth. Enzymol., 1981, 21:470-480; and Angerer et al. in Genetic Engineering: Principles and Methods (Setlow and Hollaender, Eds.) Vol. 7, pgs 43-65 (Plenum Press, New York 1985). Selection of appropriate conditions, including temperature, salt concentration, polynucleotide concentration, hybridization time, stringency of washing conditions, and the like will depend on experimental design, including source of sample, identity of capture agents, degree of complementarity expected, etc., and may be determined as a matter of routine experimentation for those of ordinary skill in the art.

Those of ordinary skill will readily recognize that alternative but comparable hybridization and wash conditions can be utilized to provide conditions of similar stringency.

After the mRNA hybridization procedure, the surface bound polynucleotides are typically washed to remove unbound nucleic acids. Washing may be performed using any convenient washing protocol, where the washing conditions are typically stringent, as described above. The hybridization of the target mRNAs to the probes is then detected using standard techniques.

In some embodiments, other methods, such as PCR-based methods, can also be used to follow the expression of the genes. Examples of PCR methods can be found in the literature. Examples of PCR assays can be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

In some embodiments, Real-Time Reverse Transcription-PCR (RT-qPCR) can be used for both the detection and quantification of RNA targets (Bustin, et al., Clin. Sci., 2005, 109:365-379). Quantitative results obtained by RT-qPCR are generally more informative than qualitative data. Thus, in some embodiments, RT-qPCR-based assays can be useful to measure mRNA levels during cell-based assays. The RT-qPCR method is also useful to monitor patient therapy. Examples of RT-qPCR-based methods can be found, for example, in U.S. Pat. No. 7,101,663, which is incorporated by reference herein in its entirety.

In contrast to regular reverse transcriptase-PCR and analysis by agarose gels, real-time PCR gives quantitative results. An additional advantage of real-time PCR is the relative ease and convenience of use. Instruments for real-time PCR, such as the Applied Biosystems 7500, are available commercially, as are the reagents, such as TaqMan Sequence Detection chemistry. For example, TaqMan® Gene Expression Assays can be used, following the manufacturer's instructions. These kits are pre-formulated gene expression assays for rapid, reliable detection and quantification of human, mouse and rat mRNA transcripts. An exemplary PCR program, for example, is 50° C. for 2 minutes, 95° C. for 10 minutes, 40 cycles of 95° C. for 15 seconds, then 60° C. for 1 minute.

To determine the cycle number at which the fluorescence signal associated with a particular amplicon accumulation crosses the threshold (referred to as the CT), the data can be analyzed, for example, using a 7500 Real-Time PCR System Sequence Detection software v1.3 using the comparative CT relative quantification calculation method. Using this method, the output is expressed as a fold-change of expression levels. In some embodiments, the threshold level can be selected to be automatically determined by the software. In some embodiments, the threshold level is set to be above the baseline but sufficiently low to be within the exponential growth region of an amplification curve.

In some embodiments, techniques known to one skilled in the art can be used to measure the amount of an RNA transcript(s). In some embodiments, the amount of one, two, three, four, five, or more RNA transcripts is measured using deep sequencing, such as ILLUMINA® RNASeq, ILLUMINA® next generation sequencing (NGS), ION TORRENT™ RNA next generation sequencing, 454™ pyrosequencing, or Sequencing by Oligo Ligation Detection (SOLID™). In other embodiments, the amount of multiple RNA transcripts is measured using a microarray and/or gene chip. In certain embodiments, the amount of one, two, three, or more RNA transcripts is determined by RT-PCR. In other embodiments, the amount of one, two, three, or more RNA transcripts is measured by RT-qPCR. Techniques for conducting these assays are known to one skilled in the art. In yet other embodiments, NanoString (e.g., nCounter® miRNA Expression Assays provided by NanoString® Technologies) is used for analyzing RNA transcripts.

In some embodiments, protein detection and quantitation methods can be used to measure the level of proteins. Any suitable protein quantitation method can be used. In some embodiments, antibody-based methods are used. Exemplary methods that can be used include but are not limited to immunoblotting (western blot), enzyme-linked immunosorbent assay (ELISA), immunohistochemistry, flow cytometry, cytometric bead array, mass spectroscopy, and the like. Several types of ELISA are commonly used, including direct ELISA, indirect ELISA, and sandwich ELISA.

In some embodiments, the protein level is determined by immunohistochemistry (IHC). IHC refers to a lab test that uses antibodies to test for certain antigens (markers) in a sample of tissue, and is a process of detecting antigens (e.g., proteins) in cells of a tissue section by exploiting the principle of antibodies binding specifically to antigens in biological tissues. The antibodies are usually linked to an enzyme or a fluorescent dye. Typically, when the antibodies bind to the antigen in the tissue sample, the enzyme or dye is activated, and the antigen can then be seen under a microscope. IHC can be used to help diagnose diseases, such as cancer. It may also be used to help tell the difference between different types of cancer. IHC can be used to image discrete components in tissues by using appropriately-labeled antibodies to bind specifically to their target antigens in situ. IHC makes it possible to visualize and document the high-resolution distribution and localization of specific cellular components within cells and within their proper histological context. While there are multiple approaches and permutations in IHC methodology, all of the steps involved can be generally separated into two groups: sample preparation and sample staining. In some embodiments, IHC is based on the immunostaining of thin sections of tissues attached to individual glass slides. Multiple small sections can be arranged on a single slide for comparative analysis, a format referred to as a tissue microarray. In other embodiments, IHC is performed by using high-throughput sample preparation and staining.

Samples can be viewed by either light or fluorescence microscopy. In some embodiments, antigen detection in tissue can be performed using an antibody conjugated to an enzyme (horseradish peroxidase) and utilized a colorimetric substrate that could be detected by light microscopy.

In some embodiments, the sample (e.g., a tissue from the patient) has been snap frozen in liquid nitrogen, isopentane or dry ice. In other embodiments, the sample (e.g., a tissue from the patient) has been fixed in formaldehyde and embedded in paraffin wax (FFPE). In both of the above-mentioned methods, the tissue or sections of the tissue can be mounted on slides prior to staining. In yet other embodiments, the IHC-free-floating technique may be used, where the entire IHC procedure is performed in liquid to increase antibody binding and penetration and slide mounting only takes place upon experimental completion. IHC-free-floating appears to be most popular in neuroscience research. When analysis of the tissue by electron microscopy is desired, the tissue can be embedded in acrylate resins such as glycol methacrylate (GMA), a technique referred to as IHC-resin.

In some embodiments, IHC can be performed using the method described in the Examples section below.

### 5.9 Kits

In some embodiments, provided herein is a kit predicting the responsiveness of a lymphoma patient to a cancer treatment, comprising agents for measuring the gene expression levels of a biological sample from the lymphoma patient. In some embodiments, the kit further comprises an agent (or tool) for taking a sample from a subject. In some embodiments, the kit further comprises an instruction on how to interpret or use the expression levels determined to predict if a patient has a particular subtype of lymphoma (e.g., DLBCL).

In certain embodiments, a kit comprises a reagent or reagents necessary for carrying out an assay(s) described herein, in one or more other containers. In certain embodiments, the kit comprises a solid support, and a means for detecting the RNA or protein expression of at least one biomarker in a biological sample. Such a kit may employ, for example, a dipstick, a membrane, a chip, a disk, a test strip, a filter, a microsphere, a slide, a multiwell plate, or an optical fiber. The solid support of the kit can be, for example, a plastic, silicon, a metal, a resin, glass, a membrane, a particle, a precipitate, a gel, a polymer, a sheet, a sphere, a polysaccharide, a capillary, a film, a plate, or a slide.

In some embodiments, the kit comprises, in one or more containers, components for conducting RT-PCR, RT-qPCR, deep sequencing, or a microarray such as NanoString assay. In some embodiments, the kit comprises a solid support, nucleic acids contacting the support, where the nucleic acids are complementary to at least 10, 20, 50, 100, 200, 350, or more bases of mRNA, and a means for detecting the expression of the mRNA in a biological sample.

In some embodiments, the kit comprises, in one or more containers, components for conducting assays that can determine one or more protein levels, such flow cytometry, ELISA, or HIC.

Such kits may comprise materials and reagents required for measuring RNA or protein. In some embodiments, such kits include microarrays, wherein the microarray comprises oligonucleotides and/or DNA and/or RNA fragments which hybridize to one or more of the genes identified in Table 1. In some embodiments, such kits may include primers for PCR of either the RNA product or the cDNA copy of the RNA product of the genes or subset of genes, or both. In some embodiments, such kits may include primers for PCR as well as probes for Quantitative PCR. In some embodiments, such kits may include multiple primers and multiple probes wherein some of said probes have different fluorophores so as to permit multiplexing of multiple products of a gene product or multiple gene products. In some embodiments, such kits may further include materials and reagents for creating cDNA from RNA. In some embodiments, such kits may include antibodies specific for one or more of the genes identified in Table 1. Such kits may additionally comprise materials and reagents for isolating RNA and/or proteins from a biological sample. In some embodiments, such kits may include materials and reagents for synthesizing cDNA from RNA isolated from a biological sample. In some embodiments, such kits may include, a computer program product embedded on computer readable media for predicting whether a patient is responsive to a compound as described herein. In some embodiments, the kits may include a computer program product embedded on a computer readable media along with instructions.

In some embodiments, antibody based kits can comprise, for example: (1) a first antibody (which may or may not be attached to a solid support) which binds to a peptide, polypeptide or protein of interest; and, optionally, (2) a second, different antibody which binds to either the peptide, polypeptide or protein, or the first antibody and is conjugated to a detectable label (e.g., a fluorescent label, radioactive isotope or enzyme). The antibody-based kits may also comprise beads for conducting an immunoprecipitation. Each component of the antibody-based kits is generally in its own suitable container. Thus, these kits generally comprise distinct containers suitable for each antibody. In some embodiments, the antibody-based kits may comprise instructions for performing the assay and methods for interpreting and analyzing the data resulting from the performance of the assay. In some embodiments, the kits comprise instructions for predicting whether a lymphoma (e.g., DLBCL) patient belongs to a specific subgroup of DLBCL (e.g., a high-risk subgroup of DLBCL).

In certain embodiments of the methods and kits provided herein, solid phase supports are used for purifying proteins, labeling samples, or carrying out the solid phase assays. Examples of solid phases suitable for carrying out the methods disclosed herein include beads, particles, colloids, single surfaces, tubes, multi-well plates, microtiter plates, slides, membranes, gels, and electrodes. In some embodiments, when the solid phase is a particulate material (e.g., a bead), it is distributed in the wells of multi-well plates to allow for parallel processing of the solid phase supports.

The practice of the embodiments provided herein will employ, unless otherwise indicated, conventional techniques of molecular biology, microbiology, and immunology, which are within the skill of those working in the art. Such techniques are explained fully in the literature. Examples of particularly suitable texts for consultation include the following: Sambrook et al., Molecular Cloning: A Laboratory Manual (2d ed. 1989); Glover, ed., DNA Cloning, Volumes I and II (1985); Gait, ed., Oligonucleotide Synthesis (1984); Hames & Higgins, eds., Nucleic Acid Hybridization (1984); Hames & Higgins, eds., Transcription and Translation (1984); Freshney, ed., Animal Cell Culture: Immobilized Cells and Enzymes (IRL Press, 1986); Immunochemical Methods in Cell and Molecular Biology (Academic Press, London); Scopes, Protein Purification: Principles and Practice (Springer Verlag, N.Y., 2d ed. 1987); and Weir & Blackwell, eds., Handbook of Experimental Immunology, Volumes I-IV (1986).

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

Certain embodiments of the invention are illustrated by the following non-limiting examples.

### 6. EMBODIMENTS

This invention provides the following non-limiting embodiments:

1. A method for predicting the responsiveness of a lymphoma patient to a cancer treatment comprising:
(a) clustering reference lymphoma patients in a reference patient group into subgroups using the expression level of at least one gene in reference biological samples of the reference lymphoma patients;
(b) determining a subgroup to which the lymphoma patient belongs based on the expression level of the at least one gene in a biological sample from the lymphoma patient; and
(c) predicting the responsiveness of the lymphoma patient to a first cancer treatment based on the subgroup of the lymphoma patient.

2. The method of embodiment 1, further comprising administering to the lymphoma patient a second cancer treatment.

3. The method of embodiment 1 or 2, wherein step (a) comprises generating clustering information defining relationships between the expression level of the at least one gene in the reference biological samples, and rearranging a heat map representation based on the clustering information.

4. The method of any one of embodiments 1-3, wherein step (a) uses a hierarchical method or a non-hierarchical method.

5. The method of any one of embodiments 1-3, wherein step (a) uses iClusterPlus method.

6. The method of any one of embodiments 1-5, wherein the reference lymphoma patients are clustered into 2-12 subgroups.

7. The method of embodiment 6, wherein the reference lymphoma patients are clustered into 7 subgroups.

8. The method of any one of embodiments 1-7, wherein the method further comprises training a classifier model using the expression level of the at least one gene in the reference biological samples.

9. The method of embodiment 8, wherein the at least one gene is selected from the genes of Table 1, optionally wherein the at least one gene comprises five or more genes of Table 1.

10. The method of embodiment 9, wherein the at least one gene comprises all genes of Table 1.

11. The method of any one of embodiments 8-10, wherein the classifier model is a grouped multinomial generalized linear model (GLM).

12. The method of any one of embodiments 8-11, wherein the classifier model is a binary model.

13. The method of any one of embodiments 8-12, wherein the method further comprises setting a threshold confidence level for at least one of the subgroups of step (a) to exclude patients that give lower confidence level clustering data from the at least one subgroup.

14. The method of any one of embodiments 1-13, wherein the lymphoma is selected from the group consisting of diffuse large B-cell lymphoma (DLBCL), indolent B cell lymphoma, follicular lymphoma, small lymphocytic lymphoma, nodal marginal zone B-cell lymphoma, lymphoplasmacytic lymphoma, anaplastic large cell lymphoma, primary cutaneous type lymphoma, mycosis fungoides, chronic lymphocytic leukemia, and mantle cell Lymphoma.

15. The method of embodiment 14, wherein the lymphoma is DLBCL.

16. The method of embodiment 14, wherein the lymphoma is indolent B cell lymphoma, nodal marginal zone B-cell lymphoma, mantle cell lymphoma, or chronic lymphocytic leukemia.

17. The method of any one of embodiments 1-16, wherein the reference patients in the reference patient group are clustered into subgroups A1-A7, and wherein:
(i) subgroup A1 comprises about 50% to about 60% patients having germinal center B-cell-like (GCB) DLBCL, about 30% to about 40% patients having activated B-cell like (ABC) DLBCL, about 10% to about 20% patients who are TME+ DLBCL patients, and about 30% to about 40% patients who are DHITsig+ DLBCL patients;
(ii) subgroup A2 comprises about 80% to about 90% patients having GCB DLBCL, about 0% to about 5% patients having ABC DLBCL, about 15% to about 25% patients who are TME+ DLBCL patients, and about 25% to about 35% patients who are DHITsig+ DLBCL patients;
(iii) subgroup A3 comprises about 40% to about 55% patients having GCB DLBCL, about 30% to about 45% patients having ABC DLBCL, about 40% to about 50% patients who are TME+ DLBCL patients, and about 20% to about 30% patients who are DHITsig+ DLBCL patients;
(iv) subgroup A4 comprises about 25% to about 35% patients having GCB DLBCL, about 40% to about 50% patients having ABC DLBCL, about 30% to about 40% patients who are TME+ DLBCL patients, and about 10% to about 20% patients who are DHITsig+ DLBCL patients;
(v) subgroup A5 comprises about 20% to about 40% patients having GCB DLBCL, about 45% to about 65% patients having ABC DLBCL, about 30% to about 40% patients who are TME+ DLBCL patients, and about 0% to about 10% patients who are DHITsig+ DLBCL patients;
(vi) subgroup A6 comprises about 30% to about 40% patients having GCB DLBCL, about 40% to about 50% patients having ABC DLBCL, about 75% to about 95% patients who are TME+ DLBCL patients, and about 0% to about 10% patients who are DHITsig+ DLBCL patients; and
(vii) subgroup A7 comprises about 0% to about 10% patients having GCB DLBCL, about 80% to about 90% patients having ABC DLBCL, about 0% to about 10% patients who are TME+ DLBCL patients, and about 0% to about 15% patients who are DHITsig+ DLBCL patients.

18. The method of any one of embodiments 1-17, wherein the first cancer treatment is a combination treatment with rituximab, cyclophosphamide, doxorubicin, vincristine, and prednisone (R-CHOP).

19. The method of any one of embodiments 1-18, wherein when the lymphoma patient is determined to belong to subgroup A1, the method comprises predicting that the patient is not likely to be responsive to the first cancer treatment.

20. The method of any one of embodiments 1-18, wherein when the lymphoma patient is determined to belong to subgroup A2, the method comprises predicting that the patient is not likely to be responsive to the first cancer treatment.

21. The method of any one of embodiments 1-18, wherein when the lymphoma patient is determined to belong to subgroup A3, the method comprises predicting that the patient is not likely to be responsive to the first cancer treatment.

22. The method of any one of embodiments 1-18, wherein when the lymphoma patient is determined to belong to subgroup A4, the method comprises predicting that the patient is not likely to be responsive to the first cancer treatment.

23. The method of any one of embodiments 1-18, wherein when the lymphoma patient is determined to belong to subgroup A5, the method comprises predicting that the patient is not likely to be responsive to the first cancer treatment.

24. The method of any one of embodiments 1-18, wherein when the lymphoma patient is determined to belong to subgroup A6, the method comprises predicting that the patient is not likely to be responsive to the first cancer treatment.

25. The method of any one of embodiments 1-18, wherein when the lymphoma patient is determined to belong to subgroup A7, the method comprises predicting that the patient is not likely to be responsive to the first cancer treatment.

26. The method of any one of embodiments 2-25, wherein the second cancer treatment is R-CHOP.

27. The method of any one of embodiments 2-25, wherein the second cancer treatment is not R-CHOP.

28. The method of embodiment 27, wherein the second cancer treatment is a bromodomain and extra-terminal (BET) inhibitor, or a cyclin dependent kinase (CDK) inhibitor.

29. A method for predicting the responsiveness of a lymphoma patient to a cancer treatment comprising:
(a) determining the expression level of at least one gene of Table 1 in a biological sample from a lymphoma patient, optionally wherein the at least one gene comprises five or more genes of Table 1;
(b) comparing the expression level of the at least one gene of step (a) with the expression level of the at least one gene in a reference biological sample from a reference lymphoma patient, wherein the reference lymphoma patient is responsive to the cancer treatment, and
wherein if the expression level of the at least one gene in the biological sample is similar to the expression level of the at least one gene in the reference biological sample, it indicates that the lymphoma patient is not likely to be responsive to the cancer treatment.

30. A method for predicting the responsiveness of a lymphoma patient to a cancer treatment comprising:
(a) determining the expression level of at least one gene of Table 1 in a biological sample of a lymphoma patient; and
(b) comparing the expression level of the at least one gene in the biological sample to: (i) the expression level of the at least one gene in biological samples from lymphoma patients who are responsive to the cancer treatment, and (ii) the expression level of the at least one gene in biological samples from lymphoma patients who are not responsive to the cancer treatment,
wherein if the expression level of (a) is similar to the expression level of (i), it indicates that the first lymphoma patient is likely to be responsive to the cancer treatment; and if the expression level of (a) is similar to the expression level of (ii), it indicates that the first lymphoma patient is not likely to be responsive to the cancer treatment.

31. A method of treating a lymphoma patient, comprising:
(i) identifying a lymphoma patient who is likely to be responsive to the cancer treatment according to the method of embodiment 30; and
(ii) administering to the lymphoma patient the cancer treatment.

32. A method of treating a lymphoma patient, comprising:
(i) identifying a lymphoma patient who is not likely to be responsive to the cancer treatment according to the method of embodiment 29 or 30; and
(ii) administering to the lymphoma patient an alternative cancer treatment.

33. The method of any one of embodiments 30-32, wherein the cancer treatment is R-CHOP.

34. The method of embodiment 32, wherein the alternative cancer treatment is a BET inhibitor, or a CDK inhibitor.

35. The method of any one of embodiments 28-34, wherein the lymphoma is selected from the group consisting of DLBCL, indolent B cell lymphoma, follicular lymphoma, small lymphocytic lymphoma, nodal marginal zone B-cell lymphoma, lymphoplasmacytic lymphoma, anaplastic large cell lymphoma, primary cutaneous type lymphoma, mycosis fungoides, chronic lymphocytic leukemia, and mantle cell Lymphoma.

36. The method of embodiment 35, wherein the lymphoma is DLBCL.

37. The method of embodiment 35, wherein the lymphoma is DLBCL, indolent B cell lymphoma, follicular lymphoma, nodal marginal zone B-cell lymphoma, mantle cell lymphoma, or chronic lymphocytic leukemia.

38. The method of any one of embodiments 29-37, wherein the expression levels of all genes of Table 1 are determined in (a) and compared in (b).

39. The method of any one of embodiments 1-38, wherein the biological samples are tumor biopsy samples.

40. The method of any one of embodiments 1-39, wherein determining the expression level of the at least one gene comprises detecting the presence or amount of at least one complex in the biological sample, wherein the presence or amount of the at least one complex indicates the expression level of the at least one gene.

41. The method of embodiment 40, wherein the at least one complex is a hybridization complex.

42. The method of embodiment 40, wherein the at least one complex is detectably labeled.

43. The method of any one of embodiments 1-39, wherein determining the expression level of the at least one gene comprises detecting the presence or the amount of at least one reaction product in the biological sample, wherein the presence or amount of the at least one reaction product indicates the expression level of the at least one gene.

44. The method of embodiment 43, wherein the at least one reaction product is detectably labeled.

45. The method of any one of embodiments 1-44, wherein the reference lymphoma patient is a refractory DLBCL patient, a relapsed DLBCL patient, or a newly diagnosed DLBCL patient.

46. The method of any one of embodiments 1-45, wherein the lymphoma patient is a refractory DLBCL patient, a relapsed DLBCL patient, or a newly diagnosed DLBCL patient.

47. The method of any one of embodiments 1-46, wherein the lymphoma patient is a GCB DLBCL patient or an ABC DLBCL patient.

48. The method of any one of embodiments 1-47, wherein the lymphoma patient is a DHITsig+ DLBCL patient or a DHITsig− DLBCL patient.

### 7. EXAMPLES

The examples below are carried out using standard techniques, which are well known and routine to those of skill in the art, except where otherwise described in detail. The examples are intended to be merely illustrative.

### 7.1 Example 1: Data Overview

### 7.1.1 Methodology

The Discovery cohort included the ROBUST clinical trial screening population (NCT02285062, (Nowakowski, et al., (2021), ROBUST: A Phase III Study of Lenalidomide Plus R-CHOP Versus Placebo Plus R-CHOP in Previously Untreated Patients With ABC-Type Diffuse Large B-Cell Lymphoma. Journal of Clinical Oncology.)) plus a set of commercially sourced newly diagnosed patient samples (n=1208). The validation datasets included the MER observational cohort (n=343) (Cerhan, et al., (2017), Cohort profile: the lymphoma specialized program of research excellence (SPORE) molecular epidemiology resource (MER) cohort study. International journal of epidemiology, 46(6), 1753-1754i), and REMoDL-B clinical trial (n=928 (Davies, et al., 2019)). For analysis of clinical outcome, only R-CHOP-treated patients were considered unless otherwise specified (or in the MER dataset, R-CHOP-like-treated patients, which included a small number of patients treated with MR-CHOP, R-EPOCH, ER-CHOP, RAD-RCHOP, and RCHOP/Zevalin). For comparison with LymphGen clusters, the NCI dataset was used (Schmitz et al., 2018, New England Journal of Medicine, 378(15), 1396-1407).

### 7.1.2 Unsupervised Clustering

The clustering input data consisted of normalized RNAseq gene expression features plus feature scores derived from the gene expression data. Expression features were restricted to the most variable and highest expressed genes in TPM space. The derived features consisted of GSVA signature scores (Hänzelmann, S. C. (2013). GSVA: gene set variation analysis for microarray and RNA-Seq data. BMC Bioinformatics.) including the MSigDB Hallmark and C1 pathways, as well as cell type signatures (Danziger, S. A. (2019). ADAPTS: Automated deconvolution augmentation of profiles for tissue specific cells. PLoS One, 14(11)). The iClusterPlus method (Mo Q, S. R. (2021). iClusterPlus: Integrative clustering of multi-type genomic data. R package version 1.30.0) was applied to the subset data for multiple choices of K from 2 to 12. This procedure was repeated 200 times, with cluster assignments recorded in each case. The 200 runs were then summarized using a sample-pairwise co-clustering frequency matrix, which was computed as the number of times two samples were assigned to the same cluster, divided by the number of times two samples appeared in the same run. This sample-pairwise matrix was then clustered using hierarchical clustering using the Ward method and 1 minus the co-cluster frequency as the distance metric, in order to obtain one final clustering per choice of K.

### 7.1.3 Linear Model Classification

A generalized linear model (GLM) classifier was trained on the discovery data using the consensus cluster labels (with A8 samples removed) as the gold standard. Several choices of the elastic net mixing parameter alpha were tested, with the goal of maximizing predictive performance and minimizing model complexity. The regularization parameter lambda was optimized using cross-fold validation and was selected as the minimum value that yielded a misclassification rate within one standard error of the minimum.

### 7.1.4 RNAseq Data Normalization

The MER and ROBUST datasets were reference normalized to a subset of the Discovery data, referred to as the commercial samples, which was fixed as a reference population. To do so, a sample-wise scaling was applied to TPM RNAseq data using the mean of five housekeeping genes (ISY1, R3HDM1, TRIM56, UBXN4, and WDR55). After sample-level scaling, each gene was standardized to the reference population by subtracting the reference mean and dividing by the reference standard deviation. Ultimately, the reference fixes all genes to have a mean of 0 and a variance of 1, while all other datasets were transformed to be a gene-wise Z-scoring with respect to the reference population. Because the REMoDL-B dataset was Illumina BeadArray and not RNAseq data, a self-standardization approach that used the housekeeping scaling step as described above was applied, followed by a gene-wise scaling that explicitly set each gene to have mean 0 and variance 1. This self-standardization approach is suitable for large, representative patient cohorts as applied herein, but could yield unexpected results for a small or non-representative cohort. The same self-standardization approach was applied to the NCI dataset for evaluation of the clusters with respect to the LymphGen calls.

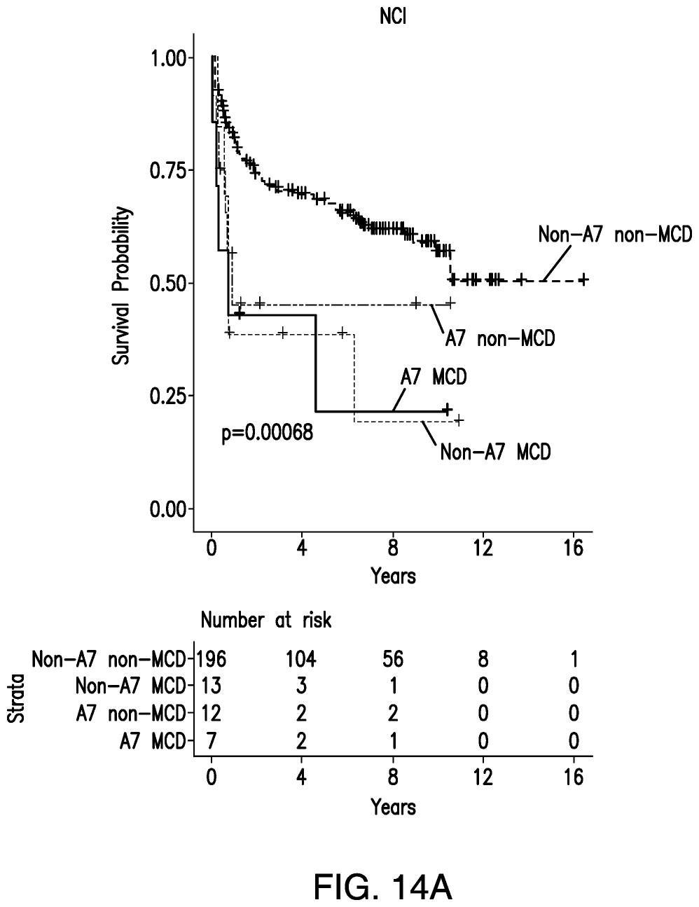

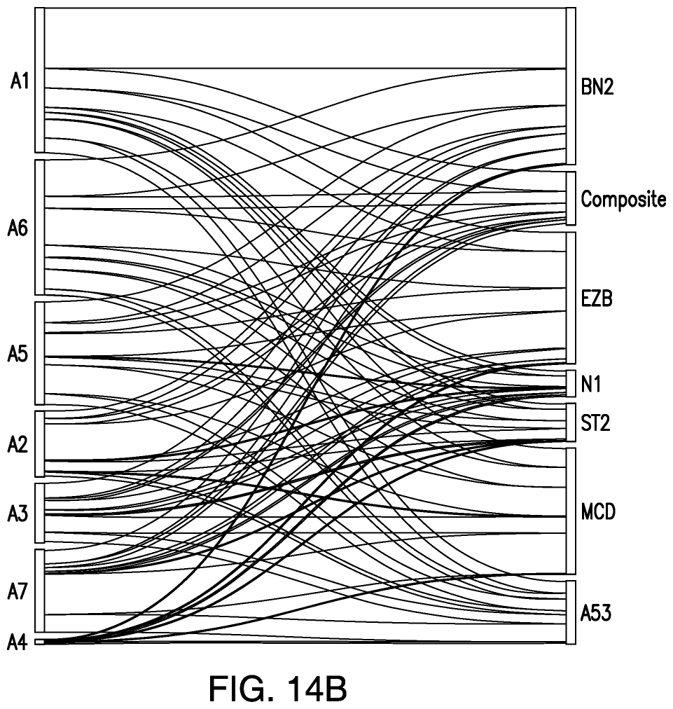

The reference normalization approach puts all of the data in a unified numerical space with comparable expression levels (FIGS. 14A-14B), and allows for portable models that can be trained in any dataset and applied directly to any other cohort without the need for re-parameterization. It also allows for the normalization of even a single sample, with no requirement for a representative batch, and furthermore, normalized data is never affected by the introduction of new samples. Existing classifiers such as the Reddy COO classifier (Reddy A, 2017, Genetic and Functional Drivers of Diffuse Large B Cell Lymphoma. Cell. 2017 Oct. 5; 171(2):481-494) and TME26 classifier were adapted to the normalized gene expression space by re-weighting decision thresholds.

In practice, no significant batch effects by dataset were observed in combined normalized cohort of all datasets. It was also validated that the normalization approach left relevant biological signals intact by comparing gene expression classifiers/signatures applied to the normalized data against orthogonal, non-RNAseq data. These included comparing the Reddy COO classification against the Hans IHC-based method, the double-hit signature classifier (Ennishi, et al., 2019, Double-hit gene expression signature defines a distinct subgroup of germinal center B-cell-like diffuse large B-cell lymphoma. Journal of Clinical Oncology, 37(3), 190) against FISH calls, and the cell type abundance GSVA scores against cell type marker density from IHC and MIBI. All features derived from the normalized RNAseq data were highly concordant with their corresponding non-RNAseq features.

### 7.1.5 Cell Type Signatures

Cell type specific signatures were generated from LM22 matrix which describes 22 functionally defined leukocyte types (Chen B, 2018, Profiling Tumor Infiltrating Immune Cells with CIBERSORT. Methods in molecular biology (Clifton, N.J.), 1711, 243-259). This signature matrix was augmented and tailored to DLBCL by adding an additional cell type representing malignant DLBCL B-cells, and was trained on purified cell populations. Benchmarking deconvolution results using the augmented signature matrix identified high correlation between abundance of the DLBCL-specific cell type and tumor purity, as well as the abundance of CD20+ cells as measured by IHC. The addition of the DLBCL-specific cell type also significantly reduced the estimated abundance of the unclassifiable “Other” cell type population, which previously accounted for up to 40% of the estimated abundance.

### 7.1.6 Sequencing

ROBUST, MER, and Commercial samples were sequenced at Expression Analysis, Inc (Durham, HC, USA) according to standard protocols. The Allprep DNA/RNA FFPE kit will be used to simultaneously purify genomic DNA and total RNA from formalin-fixed, paraffin embedded (FFPE) tissue sections. RNAseq library (75PE, 50M) was constructed using Illumina TruSeq RNA Access method.

The WES library (200× for tumor, 100× for germline control) was created using the Agilent SureSelectXT method with on-bead modifications of Fisher et al, 2011. The WGS library (60× for tumor, 30× for germline) was prepared using the Swift Accel-NGS 2S Plus DNA library kit (#21024 or 21096, Swift) with modifications to the Ampure Bead cleanup steps in the procedure.

### 7.1.7 Data Processing

Sequencing data were processed through an internal cloud-based platform. This runs the Sentieon implementation of the GATK best practices, which uses BWA-mem for alignment, and the Sentieon implementation of Mutect2 (tnhaplotyper). Variants were annotated with SnpEff using the dbnsfp database. For WGS, data copy number aberrations were called using Battenberg, and structural variants were called by Manta. For WES data, copy number aberrations were called using Sclust. Structural variants were found to be poorly represented in the WES data. RNA-seq data was aligned with STAR aligner and quantified with salmon.

7.1.8 shRNA Knockdown

Doxycycline (Dox)-inducible shRNA constructs were generated by Cellecta (Mountain View, CA, USA) using pRSITEP-U6Tet-(sh)-EF1-TetRep-2A-Puro plasmid. Briefly, 293FT cells were co-transfected with lentiviral packaging plasmid mix (Cellecta, Cat #CPCP-K2A) and pRSITEP-shRNA constructs. Viral particles were collected 48 and 72 hrs after transfection and then concentrated with Lenti-X Concentrator (Takara Bio USA). For infections, cells were incubated overnight with concentrated viral supernatants in the presence of 8 μg/ml polybrene. Cells were then washed to remove polybrene. At 48 hours post-infection, cells were selected with puromycin (2 μg/ml) for more 1 week before experiments. For knockdown experiments, cells were seeded at 1×10⁵ cells/ml and induced with 20 ng/ml of Dox or DMSO vehicle control. On day 3 of Dox induction, cells were counted and refresh with Dox or DMSO. For the proliferation assay, 15,000 cells were seeded in 96 well U-bottom plate followed by measuring cell viability with CellTiter-Glo (Promega) for 5 consecutive days. The rest of cells were seeded at 5×10⁵ cells/ml and incubated for additional 2 days. Cells were then harvested for Western blot and apoptosis assay. The shRNA target sequences were: shNT:

(SEQ ID NO: 1)
CAACAAGATGAAGAGCACCAA;
shTCF4-13:
(SEQ ID NO: 2)
GAGACTGAACGGCAATCTTTC;
shTCF4-14:
(SEQ ID NO: 3)
CACGAAATCTTCGGAGGACAA.

### 7.1.9 Western Blotting

Cells were lysed with cell lysis buffer (50 mM TrisHCl pH7.4, 250 mM NaCl, 0.5% Triton X100, 10% glycerol) supplemented with Halt protease/phosphatase inhibitors (Thermo scientific, 78443). Cell lysates were subjected to sonication to breakdown nuclei and reduce viscosity caused by released genomic DNA. The protein concentration was measured by a Bradford Protein Assay (Bio-Rad). Samples were diluted to equal concentration followed by with NuPAGE LDS sample buffer and 2-Mercaptoethanol (1.25% final concentration) before boiling at 95° C. for 5 min. Whole cell lysates were resolved on NuPAG 4-12% Bis-Tris Midi Protein Gels (Invitrogen) and transferred onto nitrocellulose membranes, which were then subjected to blocking in Intercept® (TBS) blocking buffer (LI-COR). Proteins of interest were detected by incubation with the primary antibodies listed below at 4° C. overnight. After washing with 1×TBST, the membrane was incubated with either IRDye 800CW goat anti-rabbit IgG or IRDye 680LT goat anti-mouse IgG secondary antibody (1:10,000) at RT for 1 hour. After washing with 1×TBST, bands were visualized by Odyssey Imaging System (LI-COR). Antibody information: TCF4 (Proteintech, 22337-1-AP), MYC (abcam, ab32072), GAPDH (Cell Signaling Technology, 2118L).

### 7.2 Example 2: Unsupervised Clustering of DLBCL Patients

Diffuse Large B-cell lymphoma (DLBCL) is a group of heterogeneous and aggressive germinal center B cell neoplasms and the most common form of non-Hodgkin's Lymphoma (NHL). The international prognostic index (IPI) for DLBCL predicts survival outcomes in newly diagnosed DLBCL patients based on clinical risk factors. Patients with IPI 3-5 are considered intermediate to high risk and are often used to select patients in clinical trials due to their unfavorable outcomes on of standard of care immunochemotherapy R-CHOP. IPI does not offer biological insights to elucidate therapeutic opportunities for high-risk patients, however.

Molecular classification using cell of origin (COO) has been well described with the activated B cell (ABC) subtype having heightened risk of relapse and shorter survival on R-CHOP over the germinal center B cell (GCB) subtype. Two Phase 3 randomized control trials, PHOENIX and ROBUST, did not demonstrate greater activities by ibrutinib (Younes, et al., 2019) or lenalidomide (Nowakowski, et al., 2021) combined with R-CHOP when compared to R-CHOP alone in the high-risk ABC population. Closer examination of the R-CHOP treated ABC arms indicated varied clinical outcomes, indicative of underlying disease heterogeneity in COO precluding it from being practice changing. Classification of chromosomal rearrangements involving MYC, BCL2 and/or BCL6, the so called double-hit and triple-hit patients, consistently identified a high-risk subset of GCB patients, although no therapeutics have been approved specifically for this population.

More recently, analysis of genetic features including mutations and copy number have identified novel patient clusters that build upon. COO (Chapuy, et al., 2018, Molecular subtypes of diffuse large B cell lymphoma are associated with distinct pathogenic mechanisms and outcomes. Nature medicine, 24(5), 679-690) (Schmitz et al., 2018, New England Journal of Medicine, 378(15), 1396-1407) (Wright, et al., 2020, A probabilistic classification tool for genetic subtypes of diffuse large B cell lymphoma with therapeutic implications. Cancer Cell, 37(4), 551-568) (Lacy, et al., 2020, targeted sequencing in DLBCL, molecular subtypes, and outcomes: a Haematological Malignancy Research Network report. Blood, 135(20), 1759-1771). Classifications have also incorporated the tumor microenvironment (TME26) (Risueño, et al., 2020, Leveraging gene expression subgroups to classify DLBCL patients and select for clinical benefit from a novel agent. Blood, 135(13), 1008-1018), (Kotlov, et al., 2021, Clinical and biological subtypes of B-cell lymphoma revealed by microenvironmental signatures. Cancer discovery, 11(6), 1468-1489), (Steen, et al., 2021, The landscape of tumor cell states and ecosystems in diffuse large B cell lymphoma. Cancer Cell). Despite these advances, the path to prospectively identifying high-risk newly diagnosed DLBCL patients in a practical manner that is clinically actionable with drug approvals has not been realized.

The present disclosure discovered that a robust clustering would allow for identification of biologically driven DLBCL patient subgroups and would predict patient outcome and inform treatment approaches. By defining the landscape of DLBCL subtypes in a robust and meaningful way, there could be future advances in treatment tailored to those classified subtypes. The identification of patients belonging to particular subtypes, in turn, could lead to the identification of high-risk patient groups who were underserved by current therapies (e.g., R-CHOP). Examination of the biological underpinnings of those groups could also help elucidate the mechanisms underlying the high-risk subtypes.

The present disclosure identified biologically homogeneous high-risk DLBCL patients through unsupervised clustering on transcriptomic features of both tumor and non-tumor cells. Several homogeneous clusters were identified, including one high-risk cluster described by an extreme ABC phenotype which was largely MYC pathway driven and had low immune infiltration. A gene expression classifier was developed that enabled replication of the clinical and biological characteristics in independent cohorts. Overall, the poor prognostic nature of cluster A7 and treatment-specific response profiles retrospectively in multiple randomized trials was shown suggesting that high-risk A7 had the potential to be used in clinical trials.

### 7.2.1 Discovery and Validation of Novel Clusters and Gene Expression Classifier Development

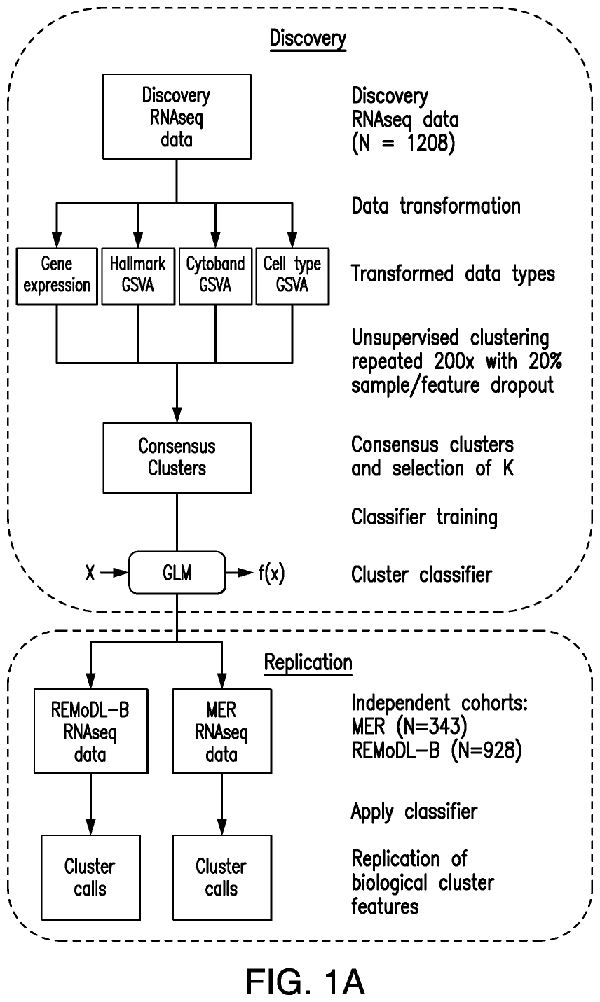

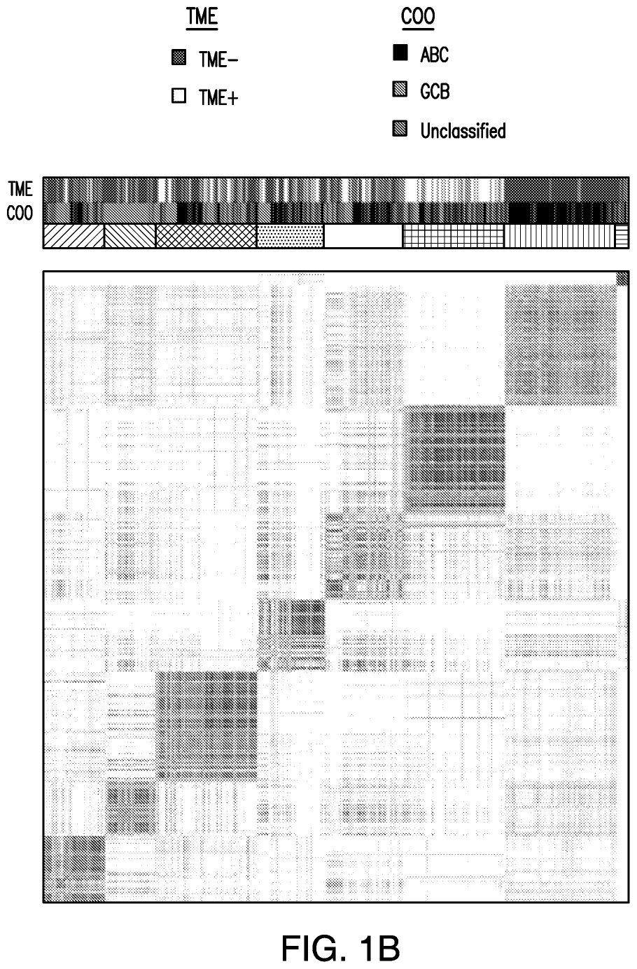

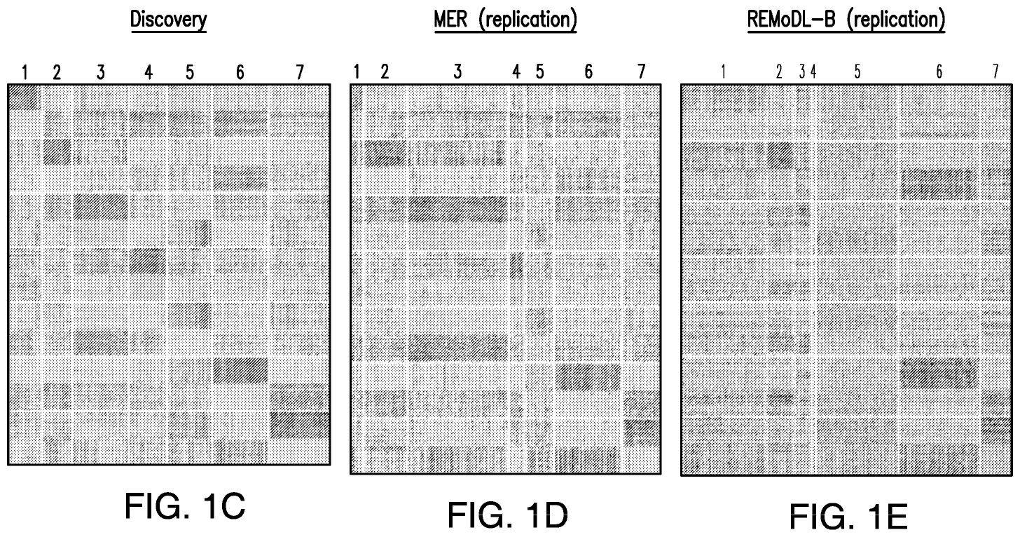

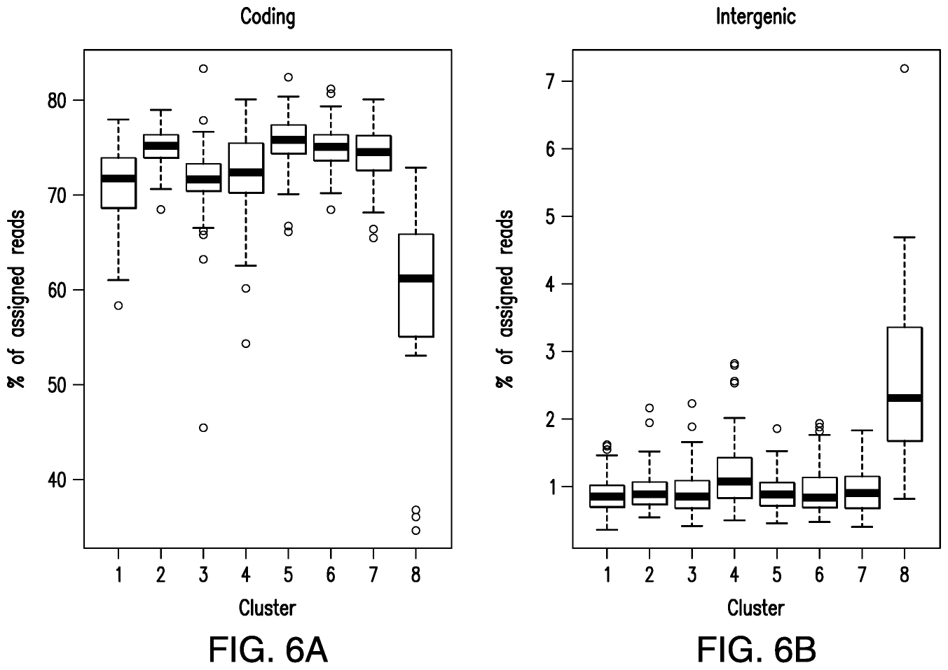

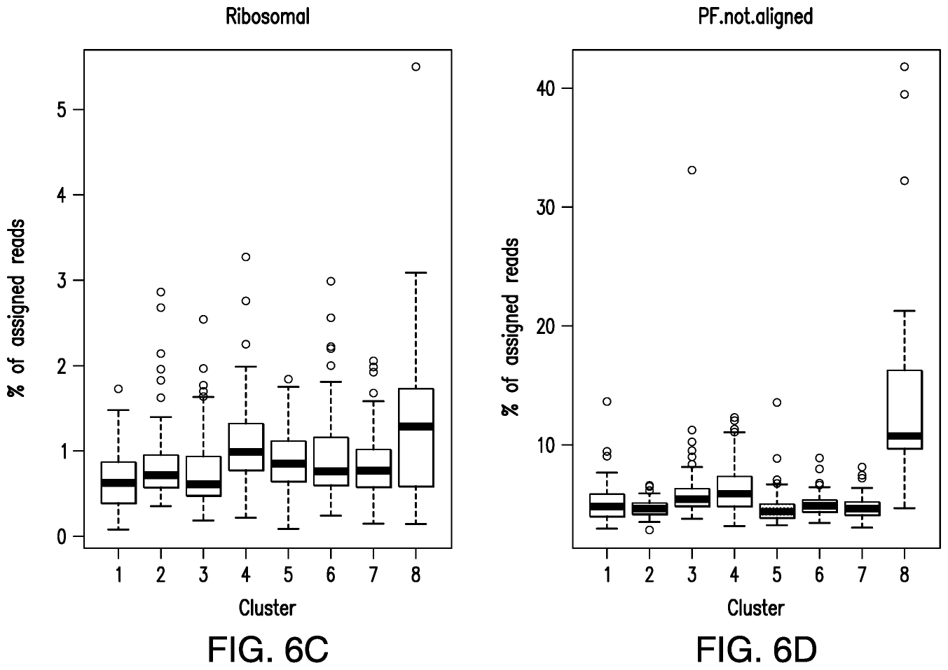

Unsupervised clustering was performed on a Discovery cohort of gene expression-derived data from newly diagnosed DLBCL patients (n=1208, Table 2), followed by supervised classifier training to identify the discovered clusters in independent datasets (FIG. 1A). The unsupervised clustering yielded 8 clusters with distinct molecular patterns (FIG. 1). These clusters had varying degrees of association with COO and TME26 classes, (Risueño, et al., 2020), but none could be uniquely determined by them. Cluster A8 was found to be a technical artifact cluster with poor alignment metrics (FIGS. 6A-6D) and was omitted from classifier training and further analysis.

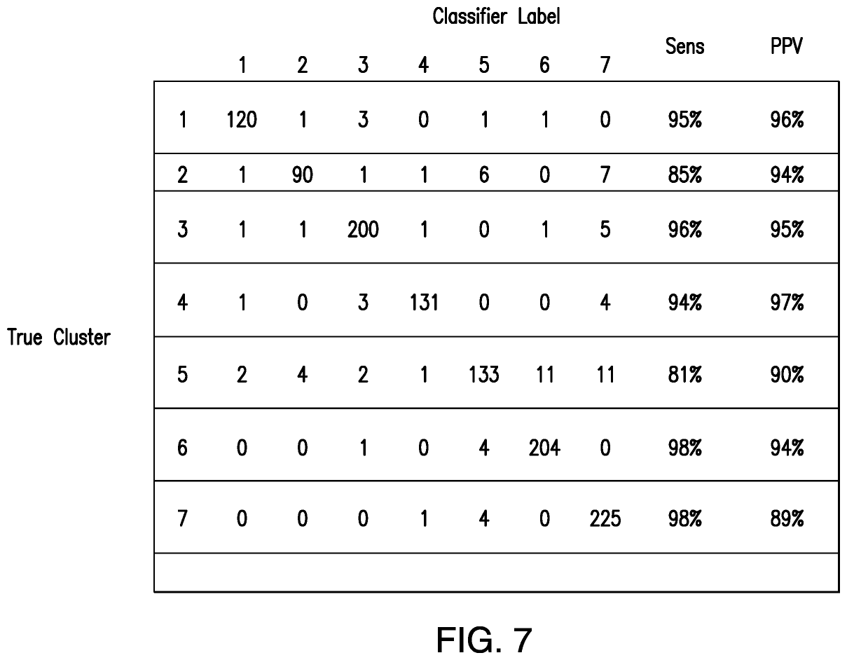

A multinomial classifier was trained on the discovery dataset to generate a model for identifying each cluster in independent validation cohorts. Cross-validation results indicated good performance of the classifier training methodology, with 93% accuracy on the training cohort, as well as 81-98% sensitivity/positive predictive value within each cluster individually (FIG. 7). Since the training data was normalized to a reference population, the classifier was directly applicable to other datasets normalized to this space, with no need to re-train parameters or thresholds. The classifier could be applied to any FFPE RNAseq sample normalized in the same way and would produce a class label for each case (i.e., no case will be unclassified).

Application of the classifier to the independent cohorts MER (validation cohort 1, n=343) (Cerhan, et al., 2017)) and REMoDL-B (validation cohort 2, n=928) (Davies, et al., 2019, Gene-expression profiling of bortezomib added to standard chemoimmunotherapy for diffuse large B-cell lymphoma (REMoDL-B): an open-label, randomised, phase 3 trial. The lancet oncology, 20(5), 649-662)) identified 7 clusters with reproducible biology, including the top 50 up/down differentially expressed genes in each cluster which show reproducible expression patterns across the clusters (FIG. 1C).

### 7.2.2 Clinical Outcome and Characteristics of High-Risk Cluster A7

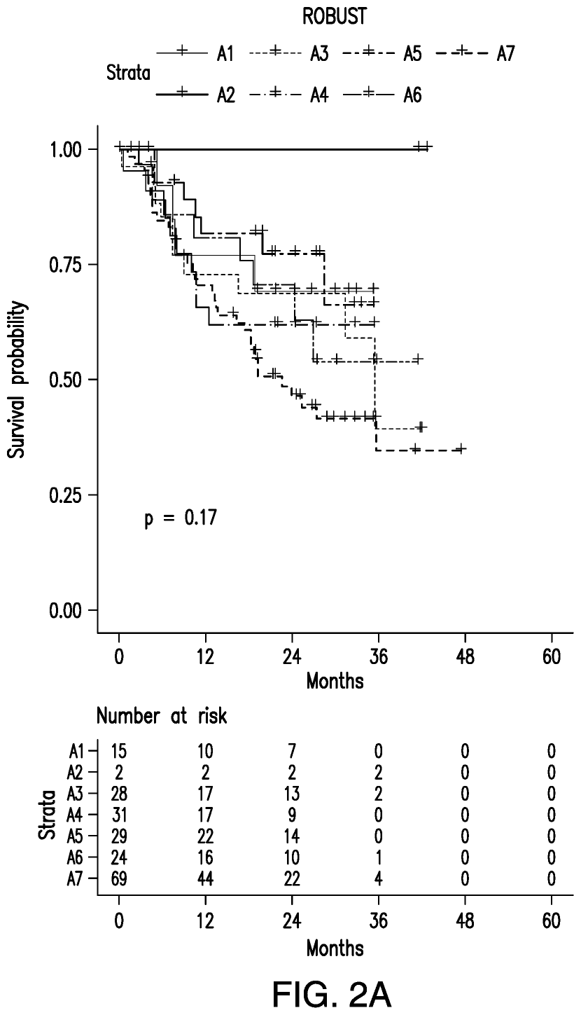

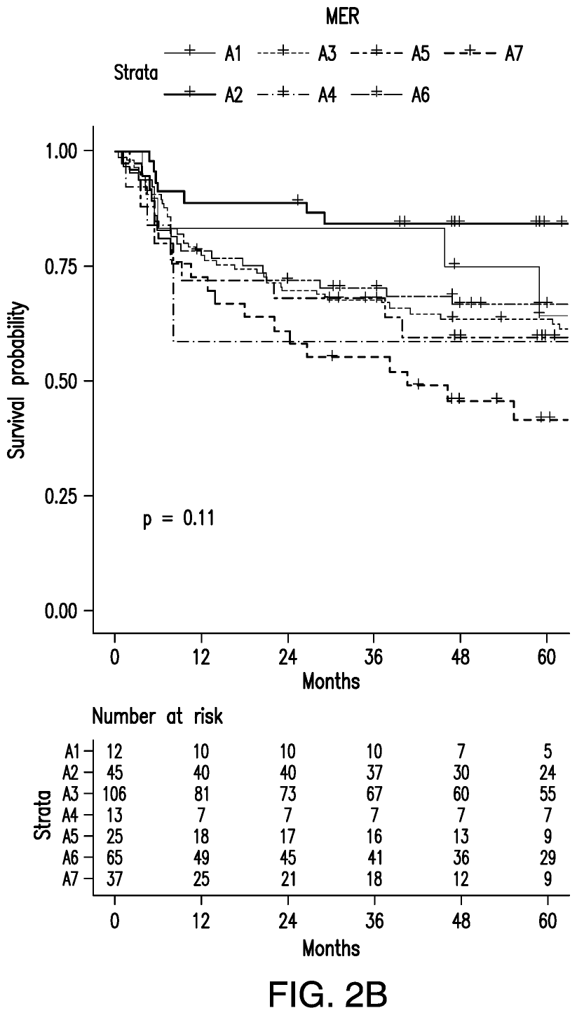

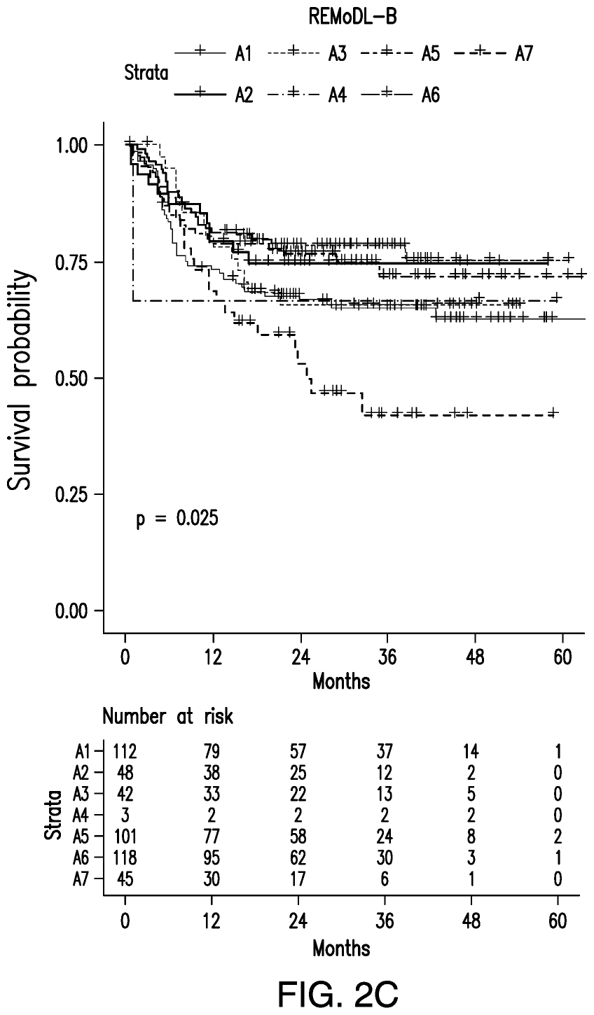

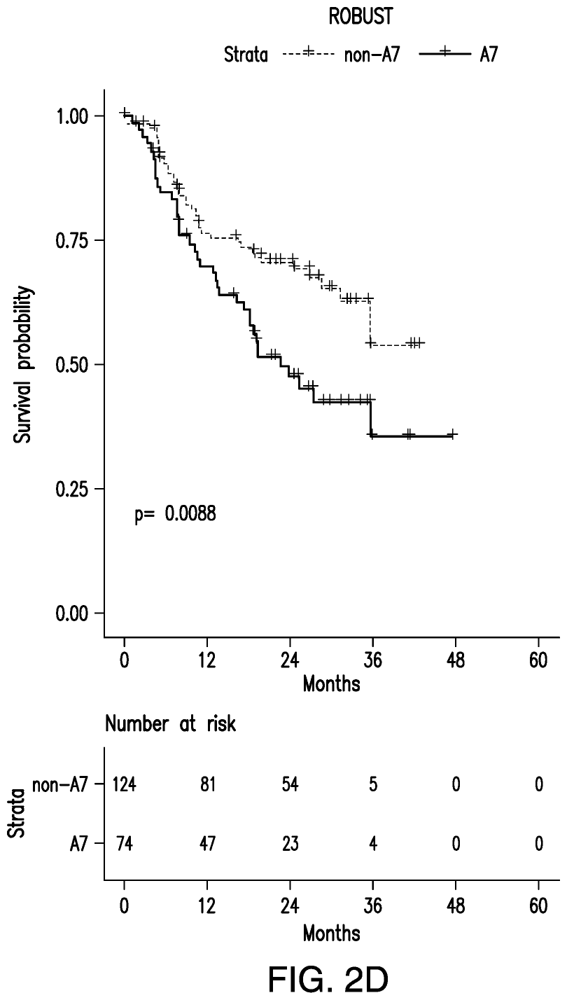

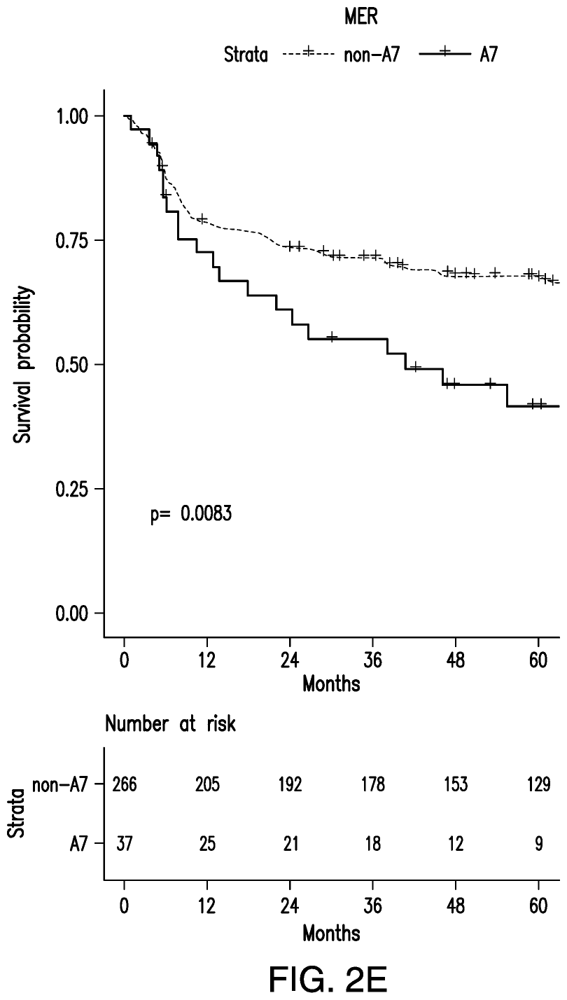

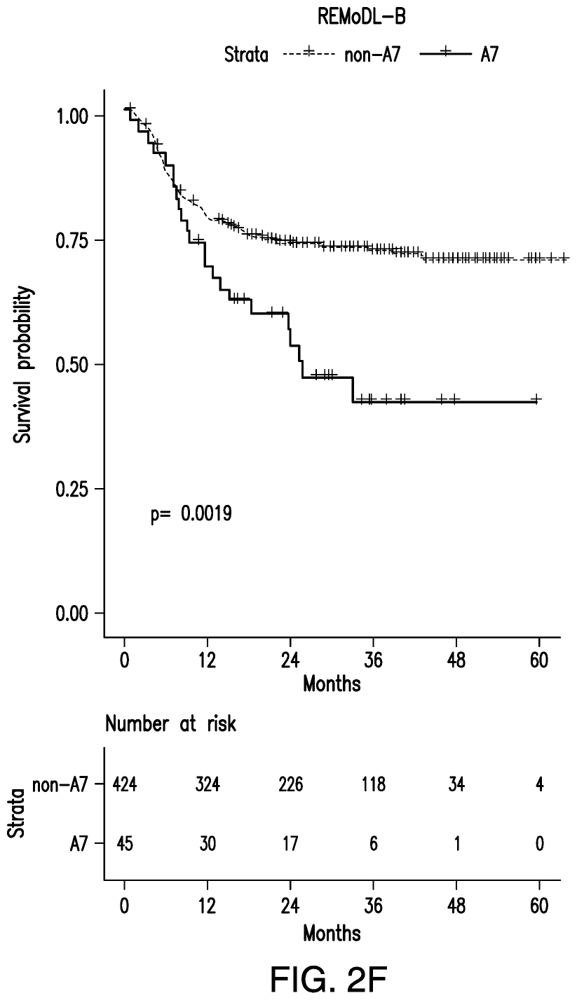

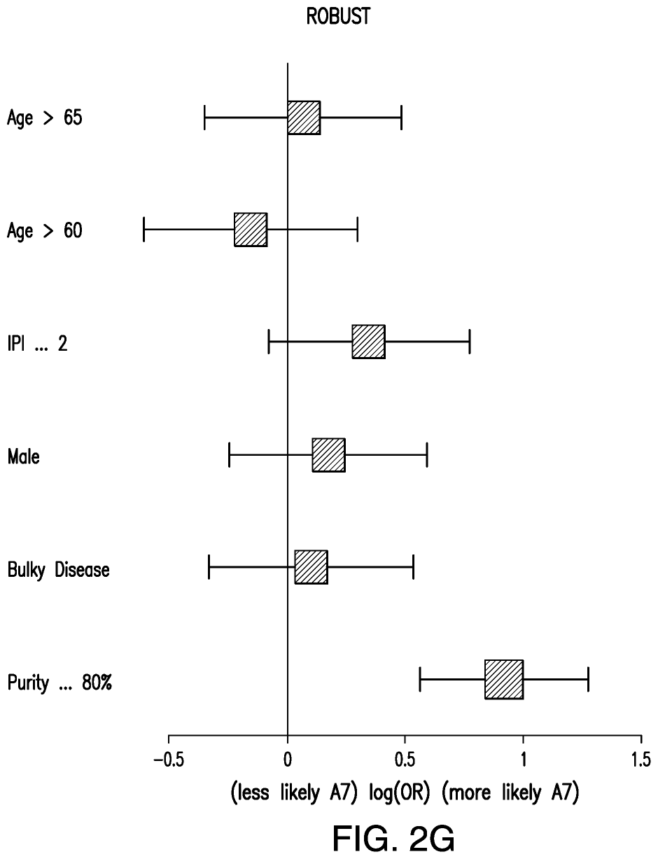

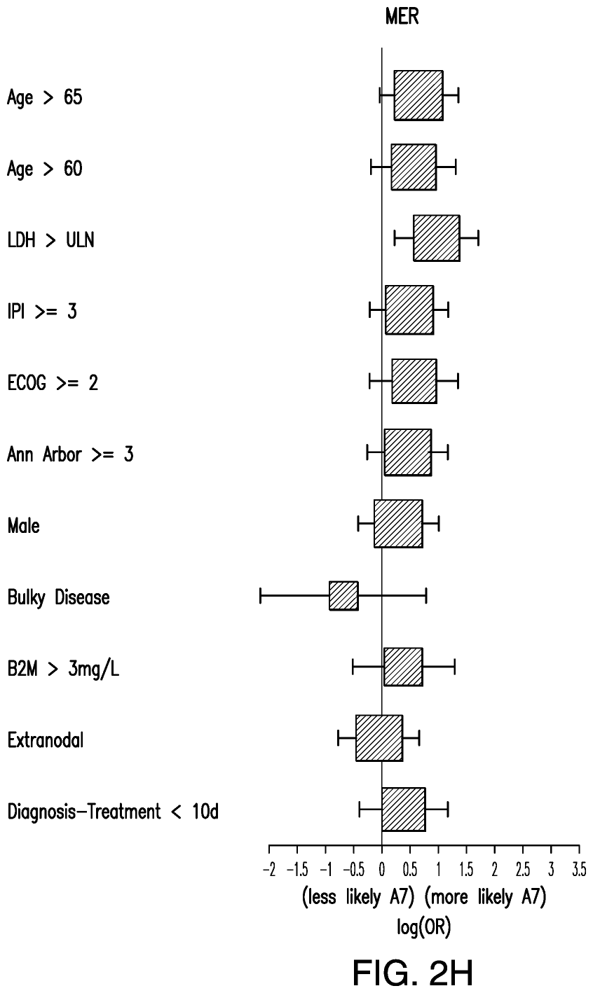

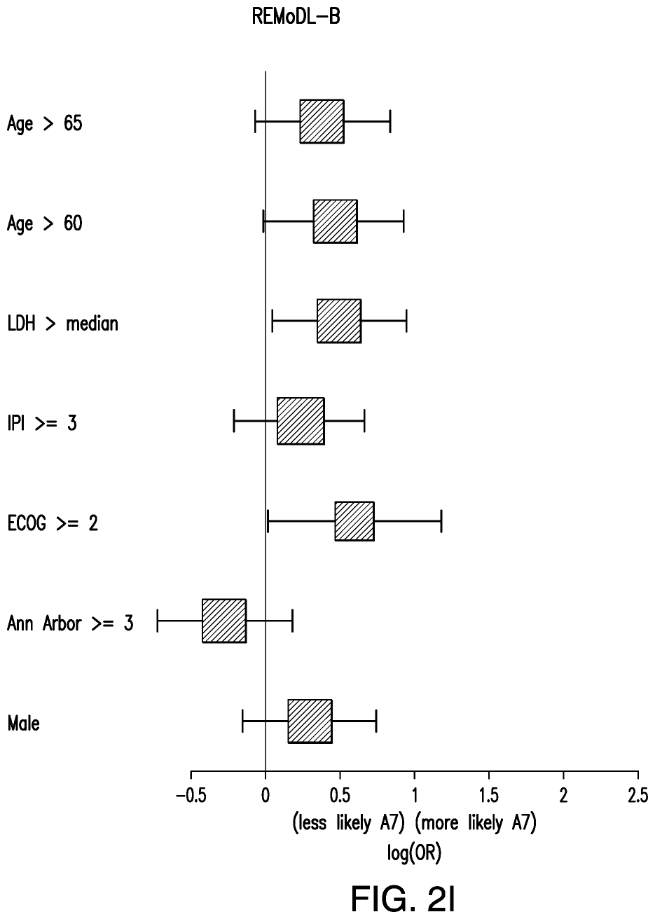

While the cluster discovery was performed in the absence of clinical outcome data, it was investigated if any cluster was associated with unfavorable prognosis. The clusters' association with survival outcome on R-CHOP and association with prognostic features was shown in FIGS. 2A-2I. Cluster A7 represented an ABC-enriched group of patients with the worst response to RCHOP among the 7 clusters, with a prevalence of 19%, 13%, and 11% in ROBUST, MER, and REMoDL-B respectively. A7 status was significantly prognostic, with A7 vs. non-A7 hazard ratios (95% confidence interval) of 1.65 (1.08-2.51), 1.87 (1.17-3.00), and 2.00 (1.23-3.20) in ROBUST (ABC only), MER, and REMoDL-B, respectively.

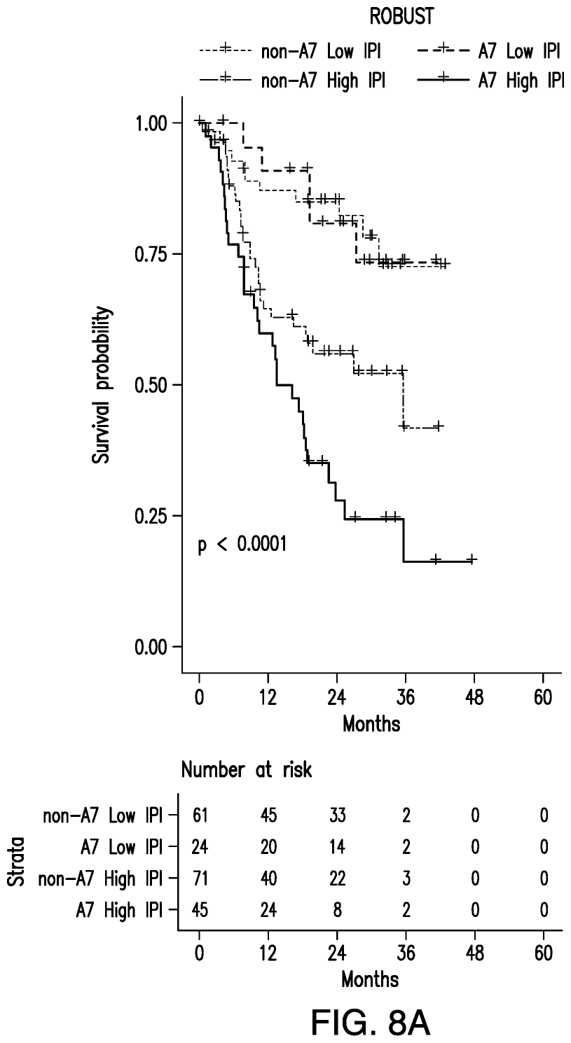

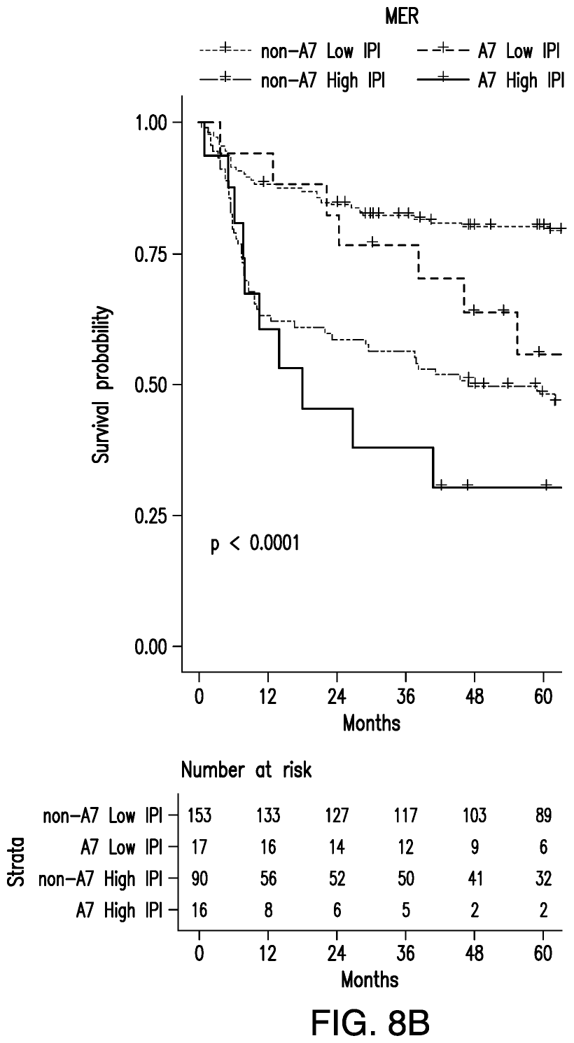

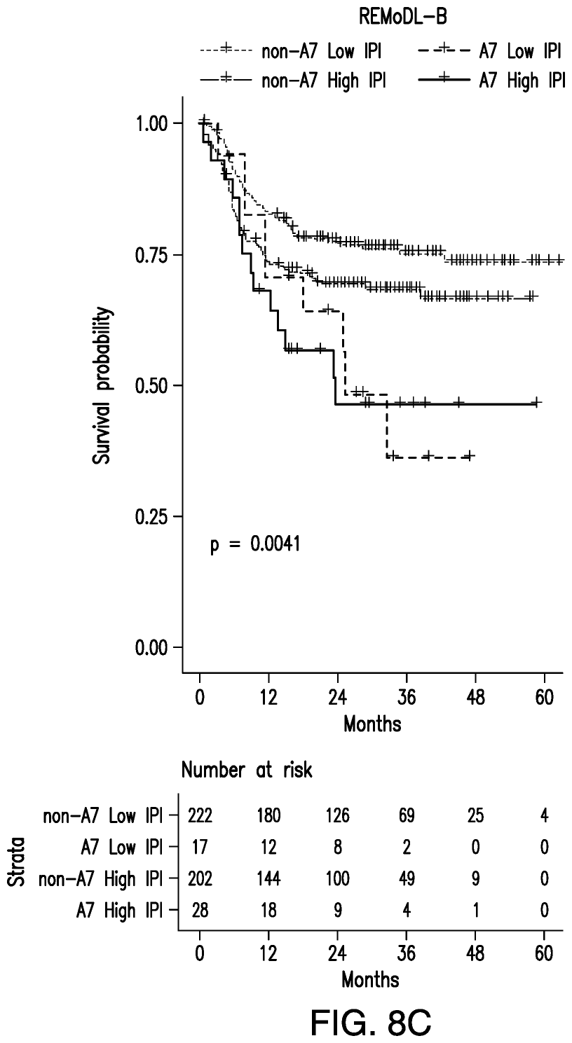

Although the ABC COO subtype was associated with elevated risk, the high-risk nature of A7 was not simply due to its ABC enrichment. Even within ABC-only populations, A7 patients were higher risk than non-A7 patients (FIGS. 2D-2F). The association test of A7 with known clinical prognostic factors indicated no strong influence by IPI or its components, indicating that A7 could not be defined using clinical features (FIGS. 2G-2I). Cox proportional hazard models showed that A7 status was a significantly prognostic factor both in a univariate model (p=0.027) as well as in a multivariate model combined with IPI (p=0.047), and that an A7+IPI model was marginally more prognostic than IPI alone (ANOVA p=0.06, FIGS. 8A-8C). Although A7 was strongly associated with both COO and TME26 (p<2.2e-16 for both), neither feature alone or combined were sufficient to uniquely identify A7. Using the COO or TME26 scores as univariate predictors of A7 membership yielded prediction AUCs between 0.82 and 0.86 in both ROBUST and MER, with optimized classifiers achieving roughly 80% sensitivity and 70% specificity in classifying A7.

### 7.2.3 Biological Interpretation of Novel Clusters

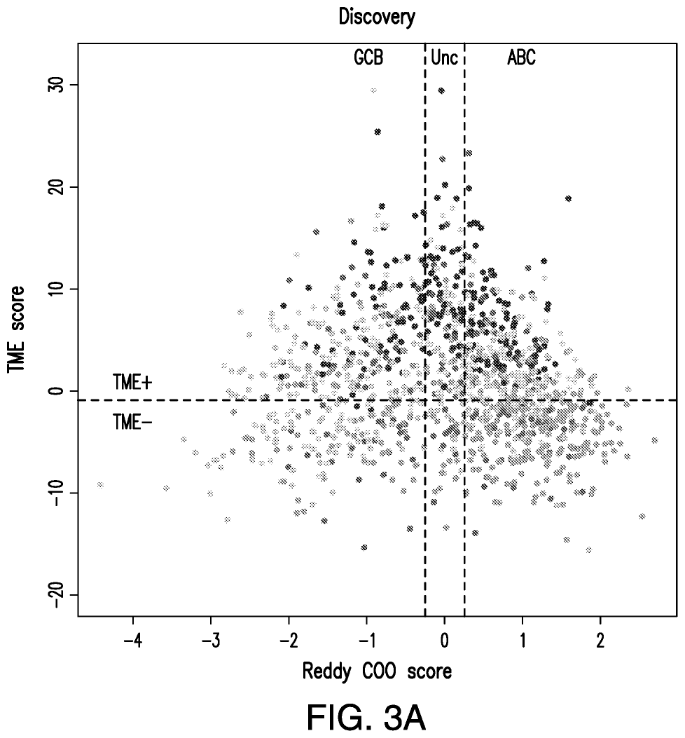

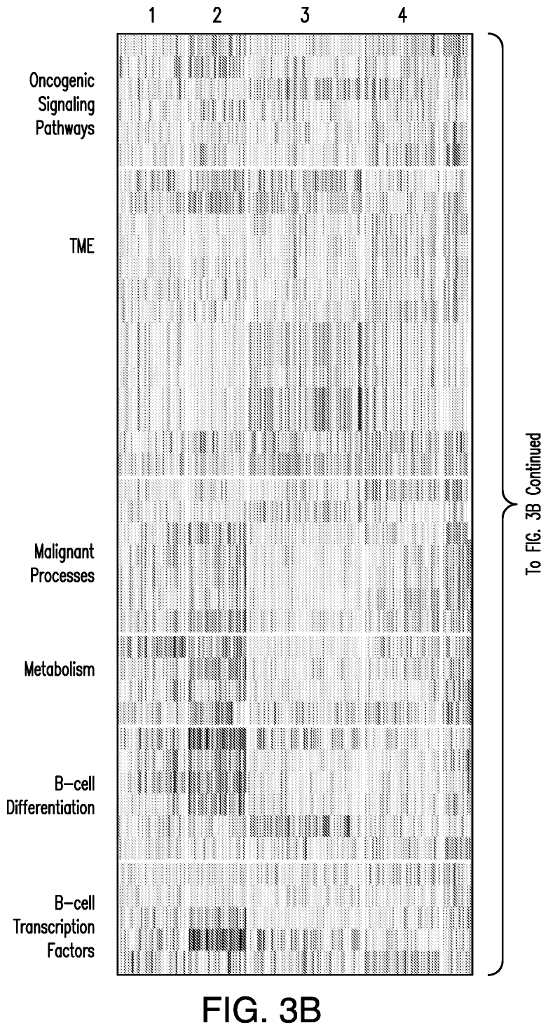

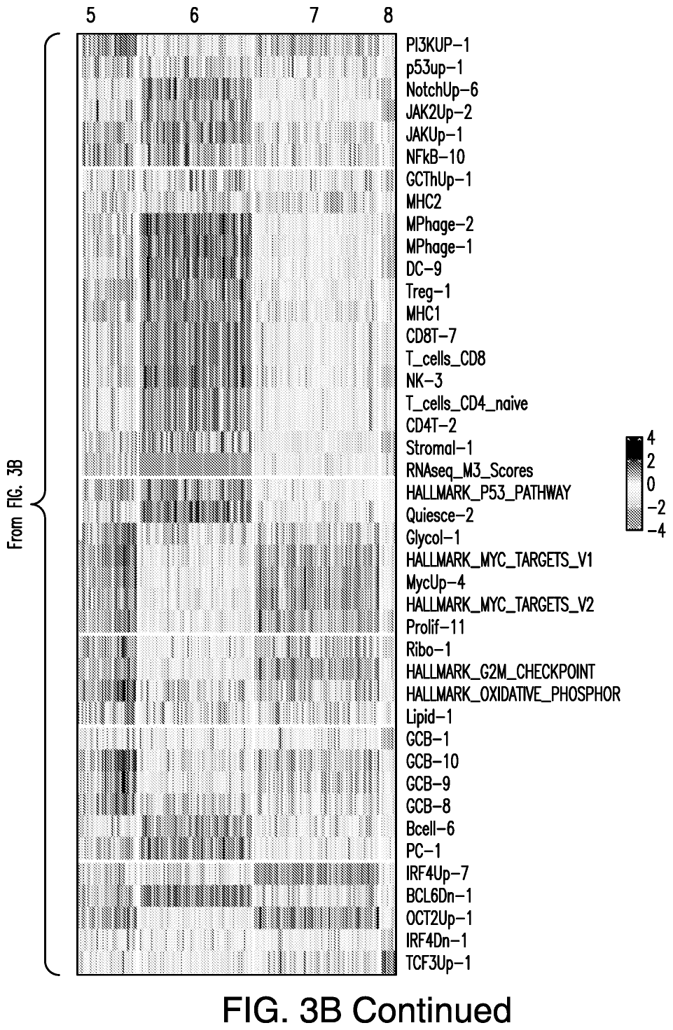

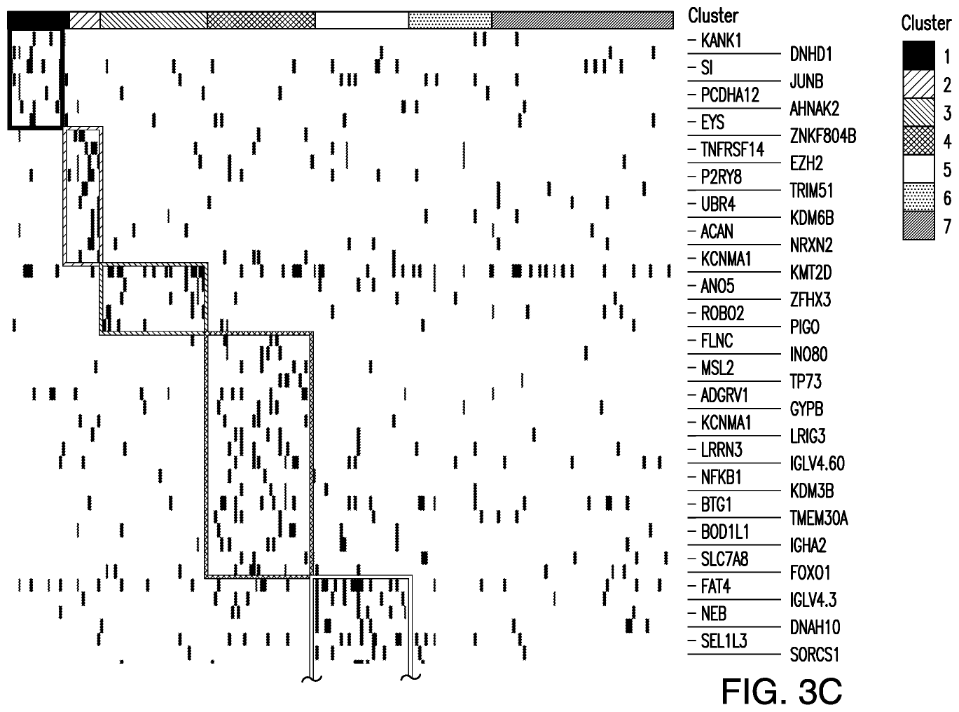

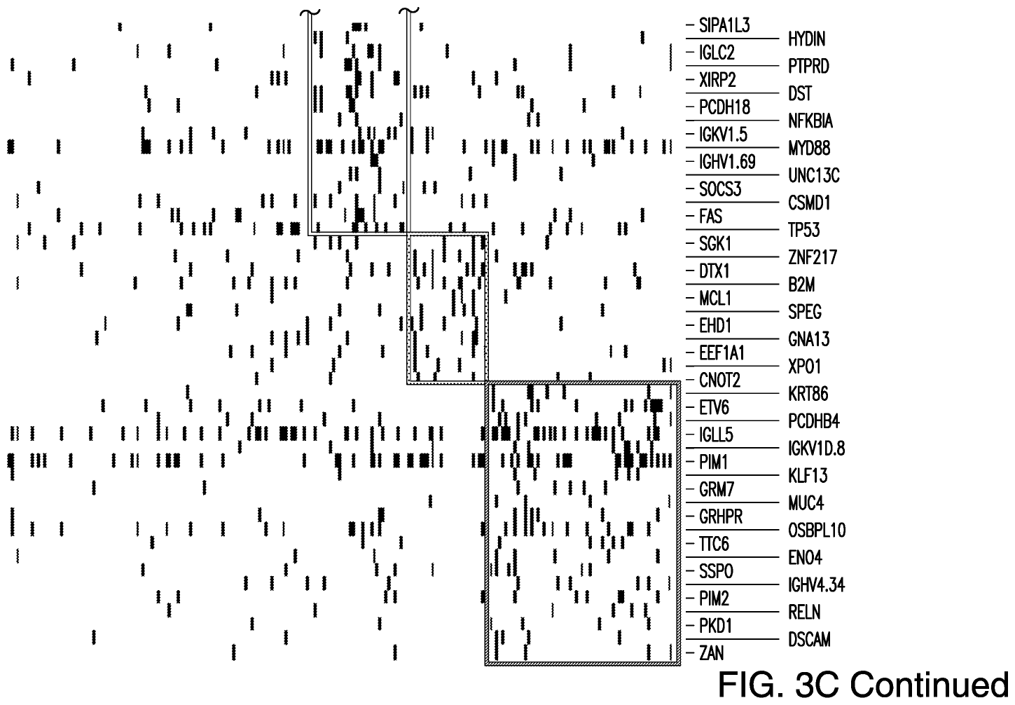

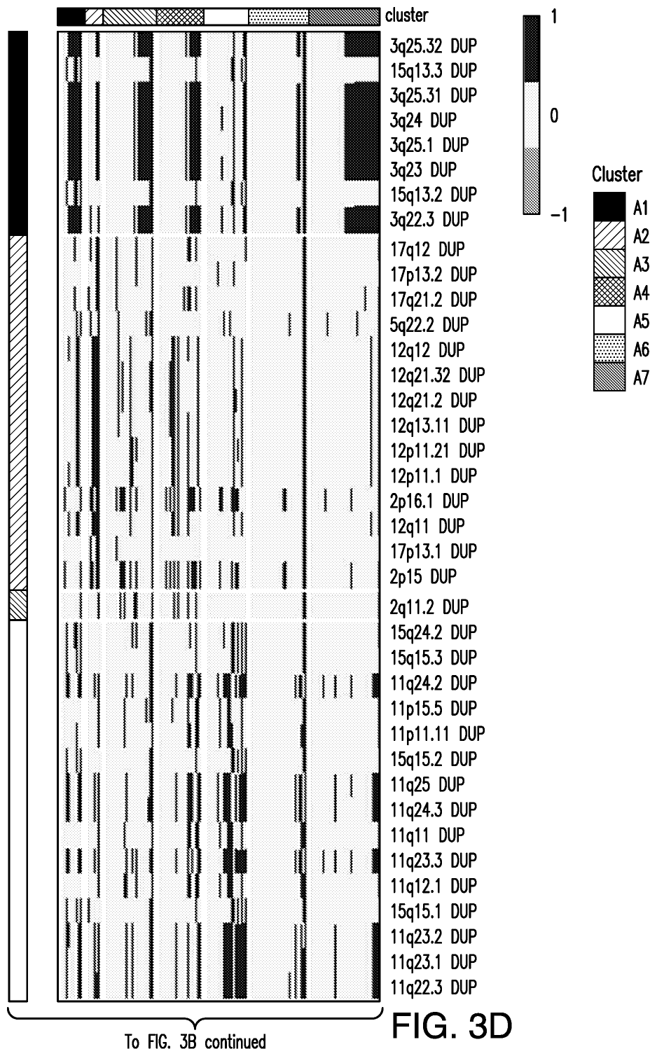

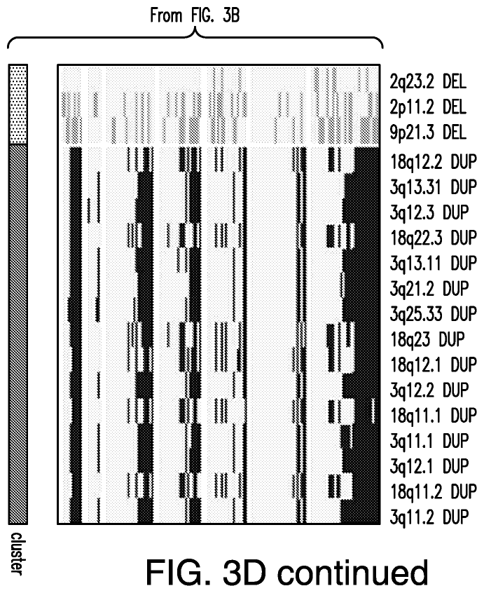

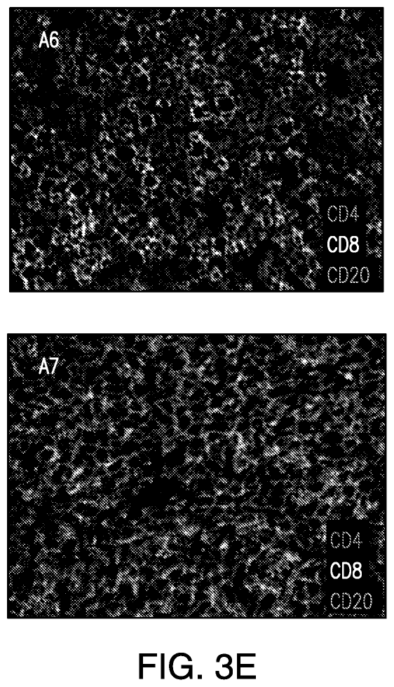

Each of the clusters was examined for differential biology in terms of single gene expression, DLBCL-specific pathways (Wright, et al., 2020), copy number aberrations, single nucleotide variants and tumor microenvironment. Distinctions among the clusters were identifiable through the lens of COO and TME26 (FIG. 3A), although significant heterogeneity remained via these dimensions. Three clusters were notably extreme in COO-TME26 space, which were the low-TME GCB-enriched cases found in A2, the low-TME ABC-enriched cases found in A7, and the high-TME Unclassified-enriched cases found in A6.

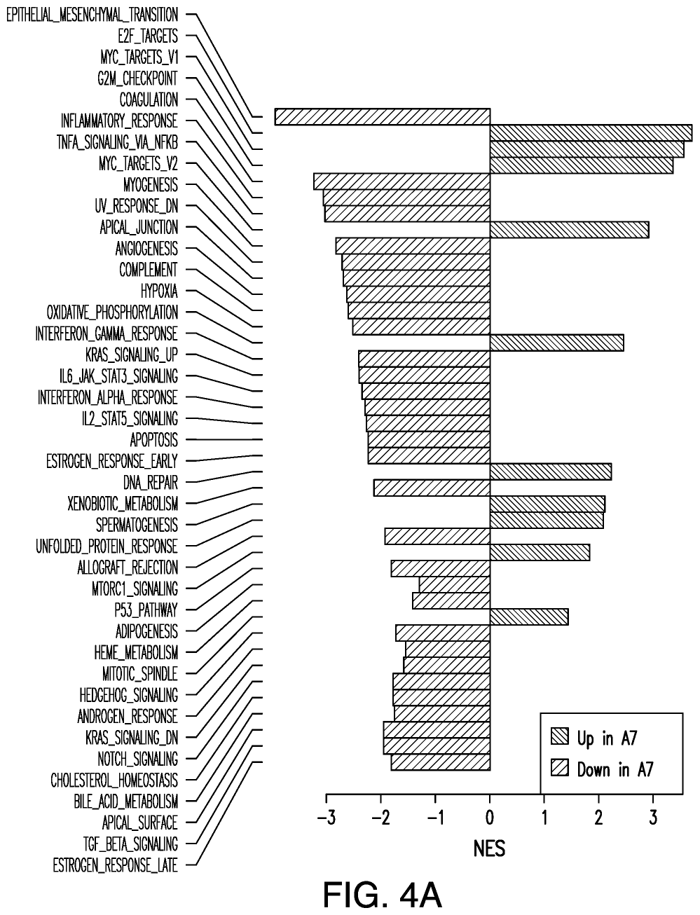

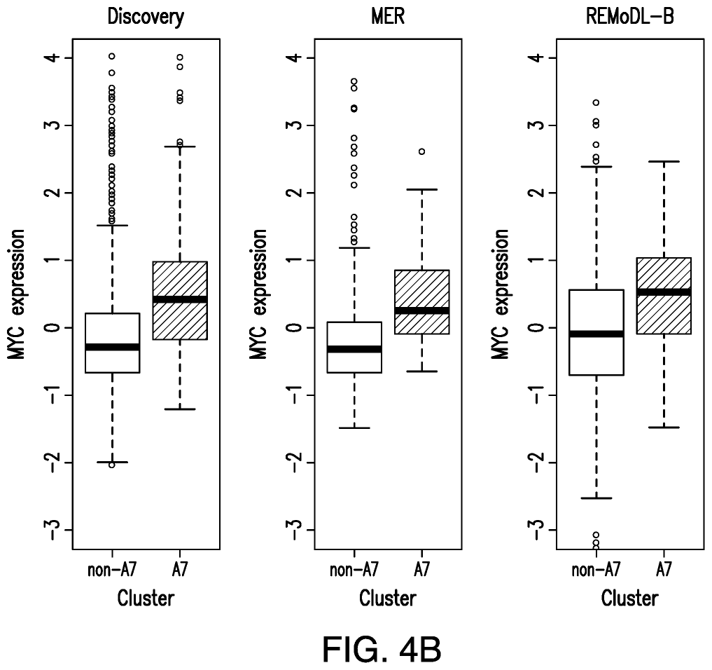

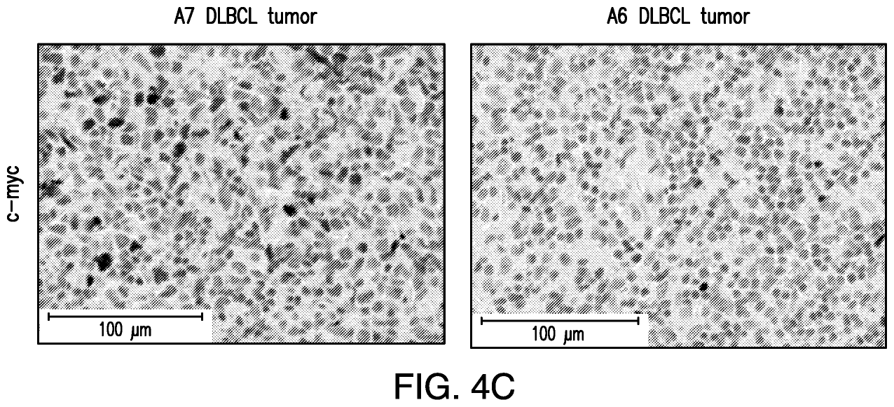

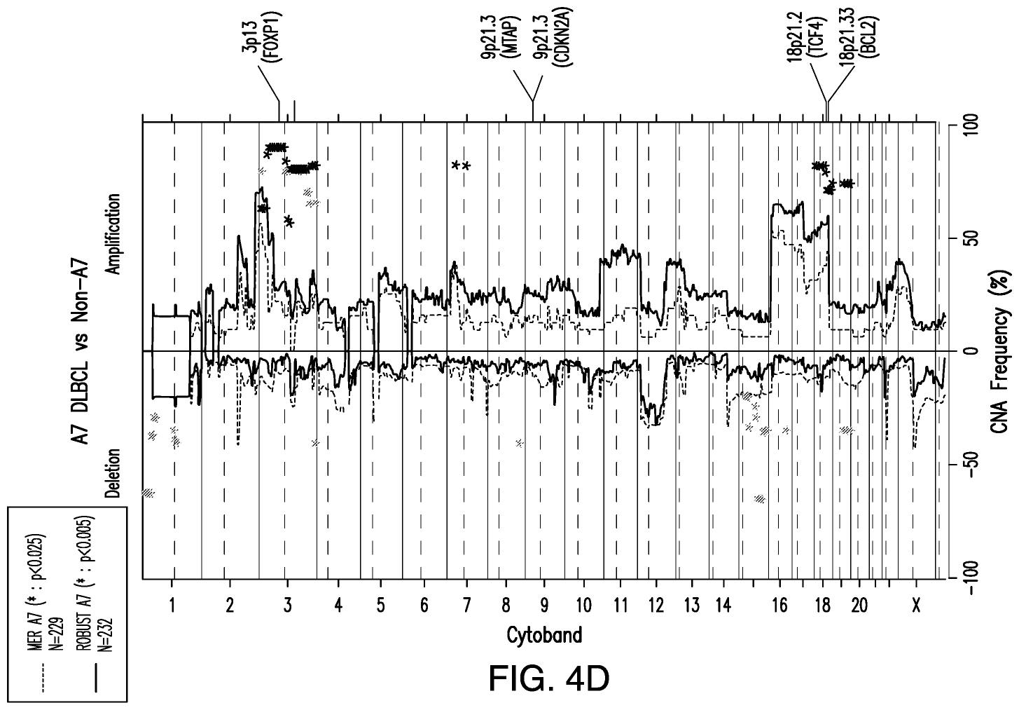

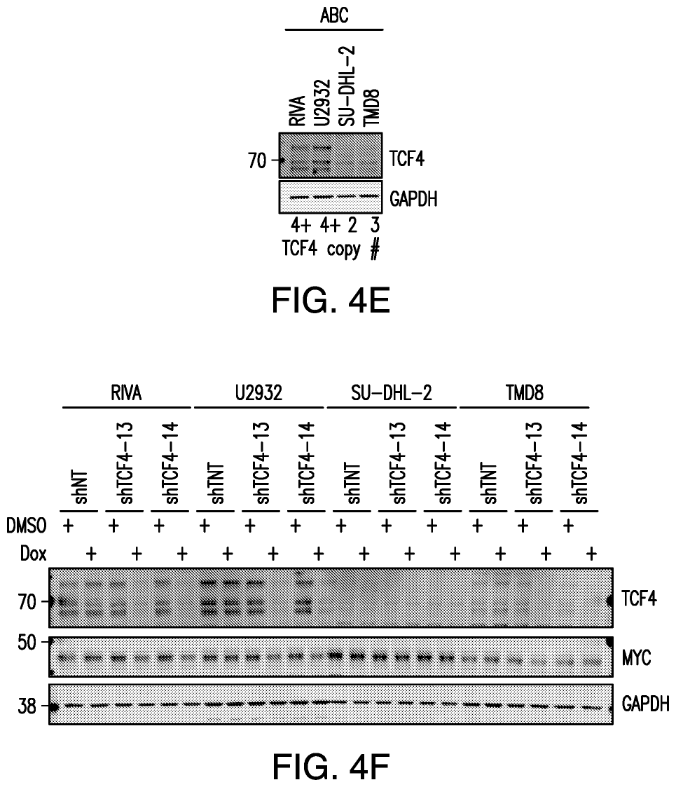

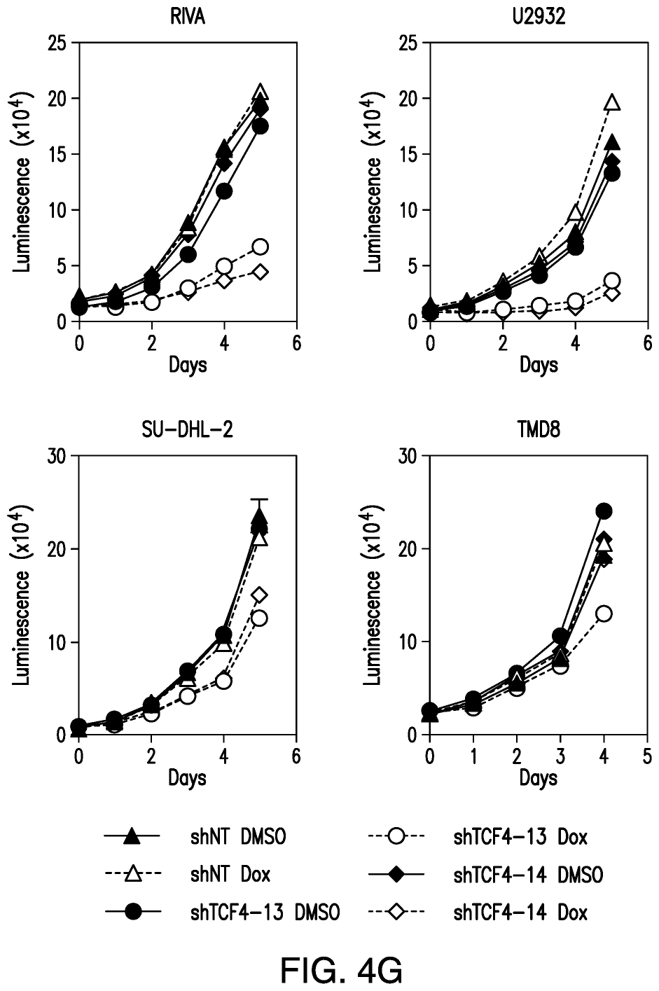

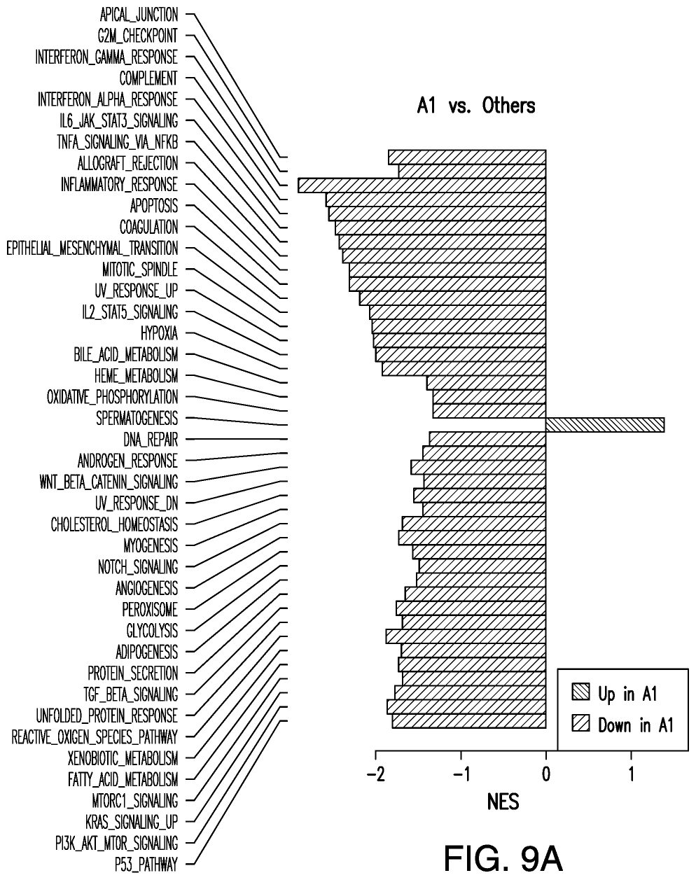

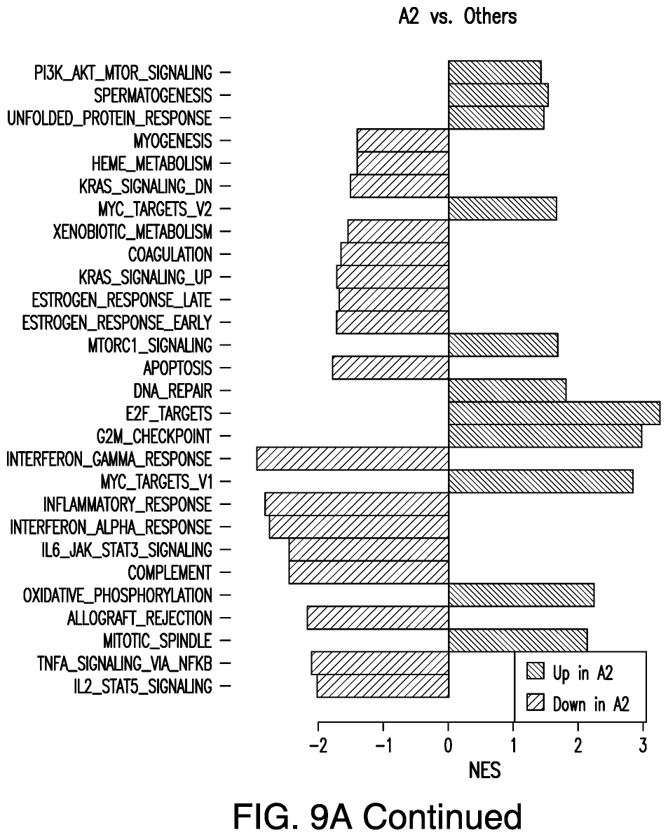

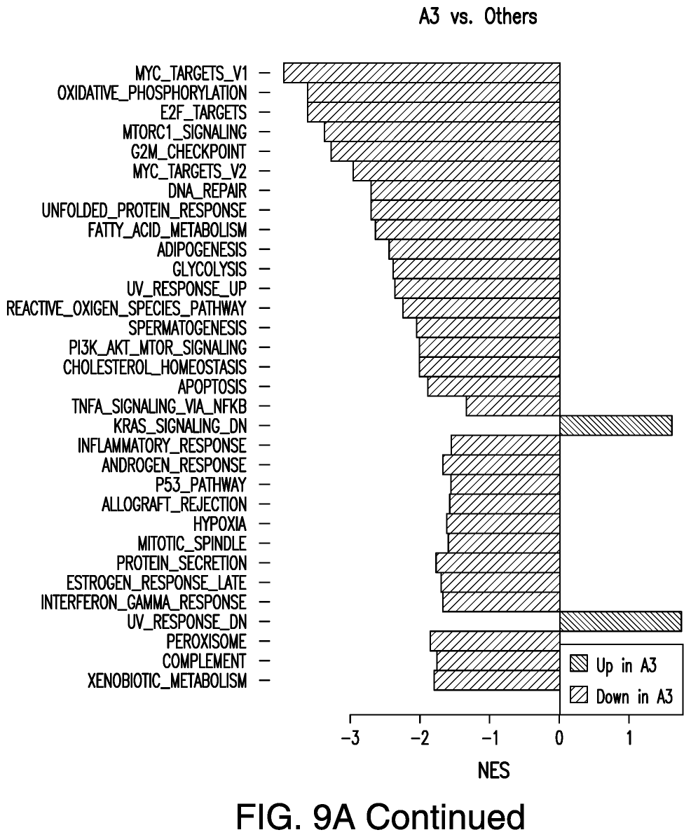

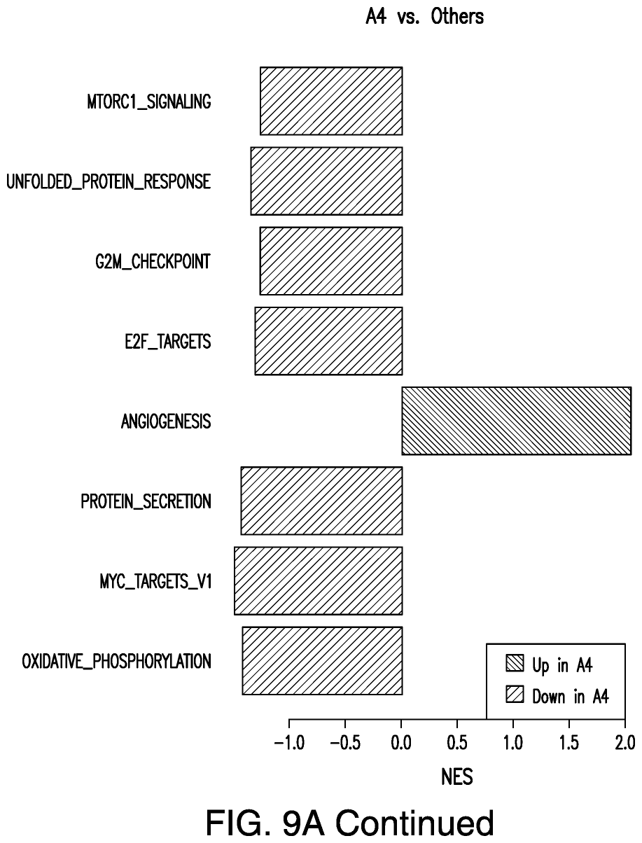

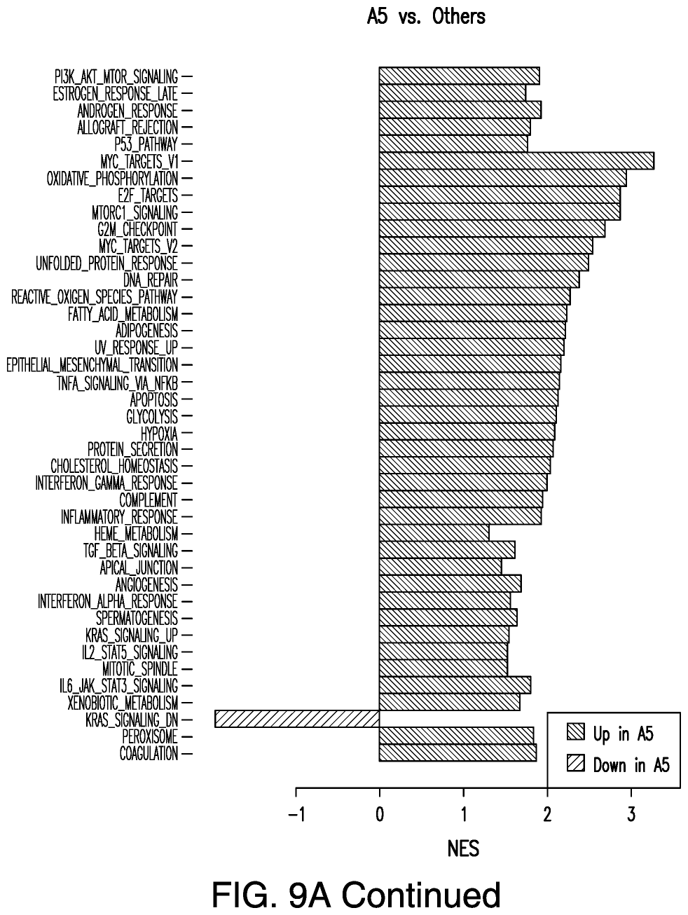

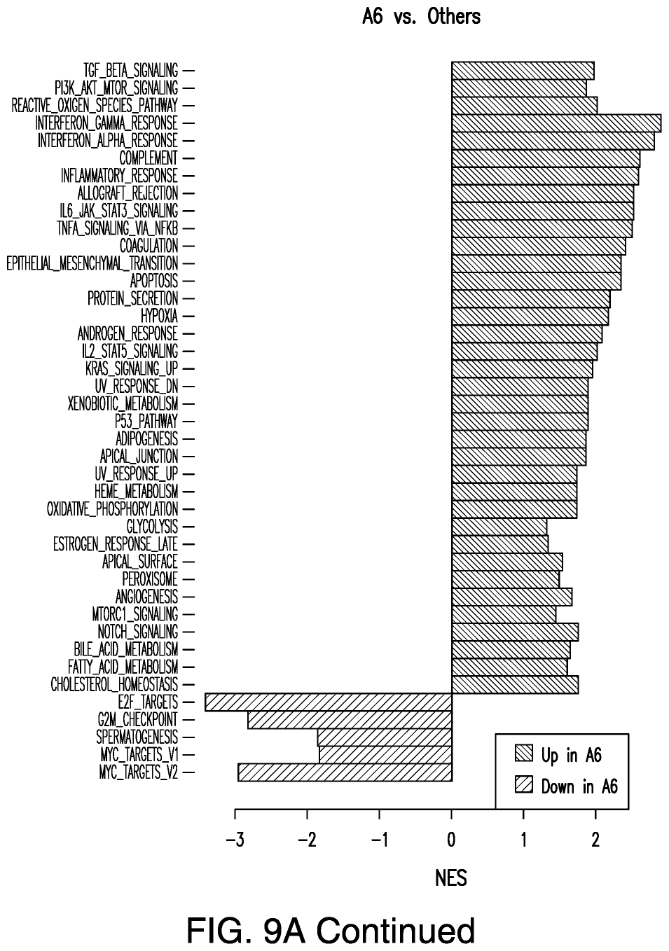

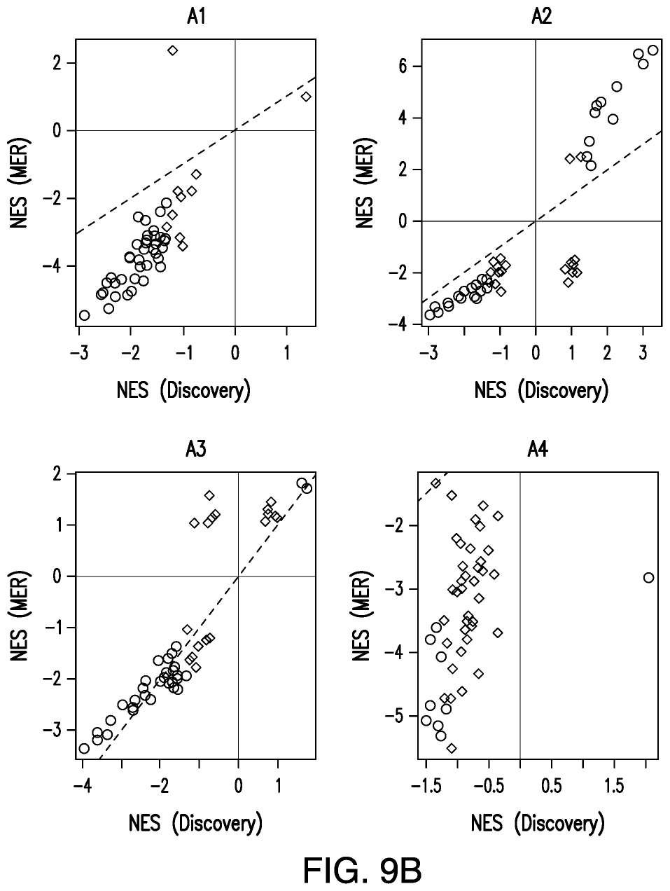

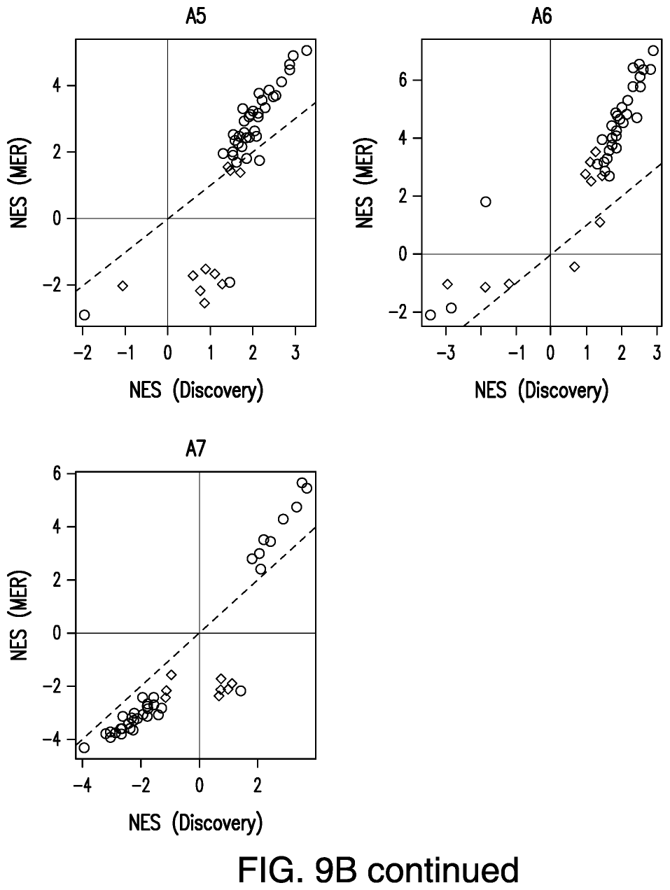

A variety of DLBCL-relevant pathways utilized in Wright et al. allowed deeper insight into pathways contributing to each cluster from the tumor microenvironment, COO, oncogenic pathways and metabolomic perspectives (FIGS. 3A-3E). Among the most distinct signals were the upregulation of a variety of immune-associated, JAK, and NFKB signatures in A6, the upregulation of GCB-associated signatures (IRF4Dn-1) in GCB-enriched A2, the relative balance of tumor microenvironment and malignant process signatures in A5, and the downregulation of PI3K, malignant process and metabolism signatures in A3. Clusters A1 and A4 exhibited less distinct gene expression signals, although both exhibited low expression of MYC and G2M checkpoint pathways. The high-risk cluster A7 had upregulation of ABC-associated signatures (IRF4Up-7) and low expression TME signatures. A7 was highly enriched for the ABC subtype (p<2.2×10¹⁶) and had the most extreme COO scores even among ABC patients according to the Reddy et al score (data not shown). It was also characterized by upregulation of signatures such as G2M checkpoint, oxidative phosphorylation, mitotic spindle, and DNA repair, as well as low expression of p53 and TME signatures (FIG. 4A). Further description of cluster-defining pathways were shown in FIGS. 9A-9B.

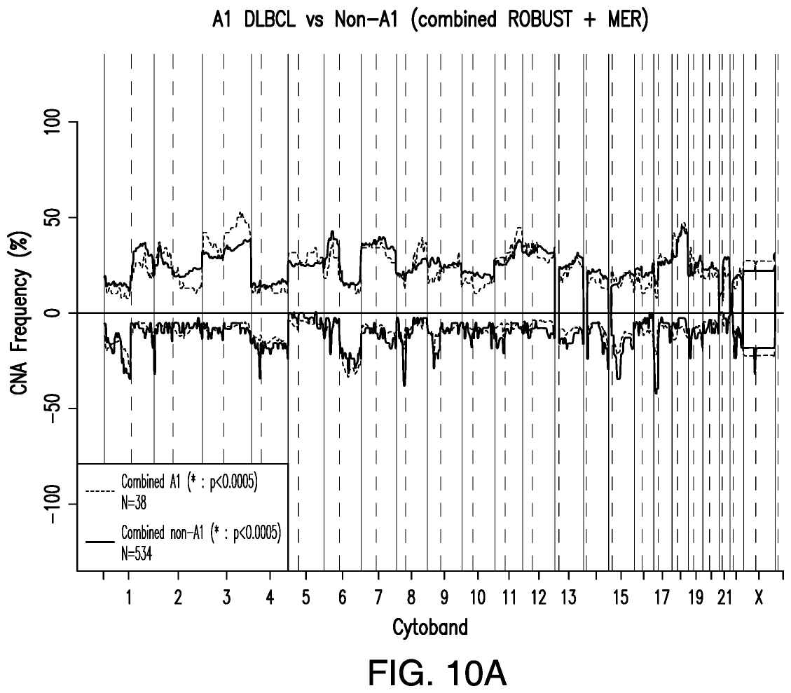

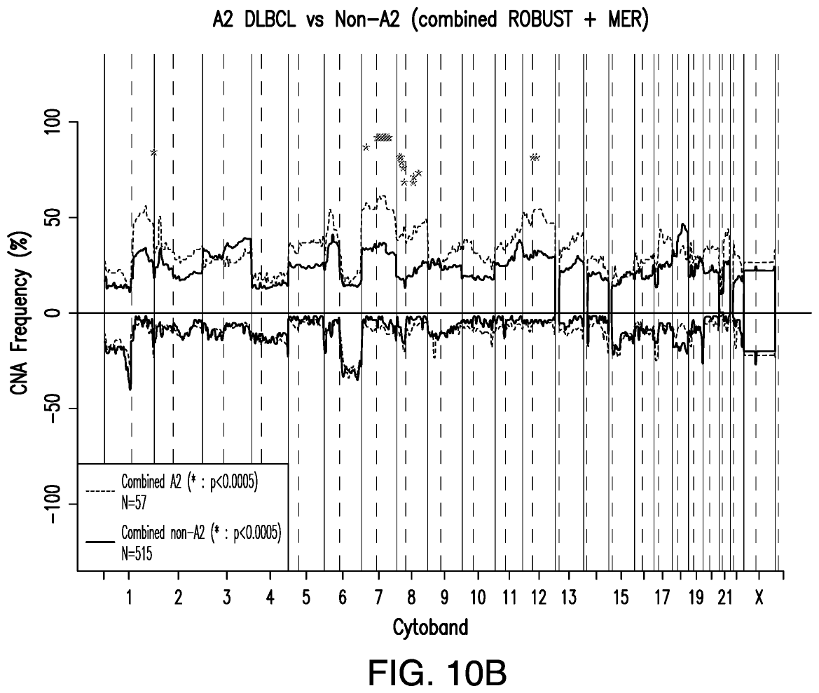

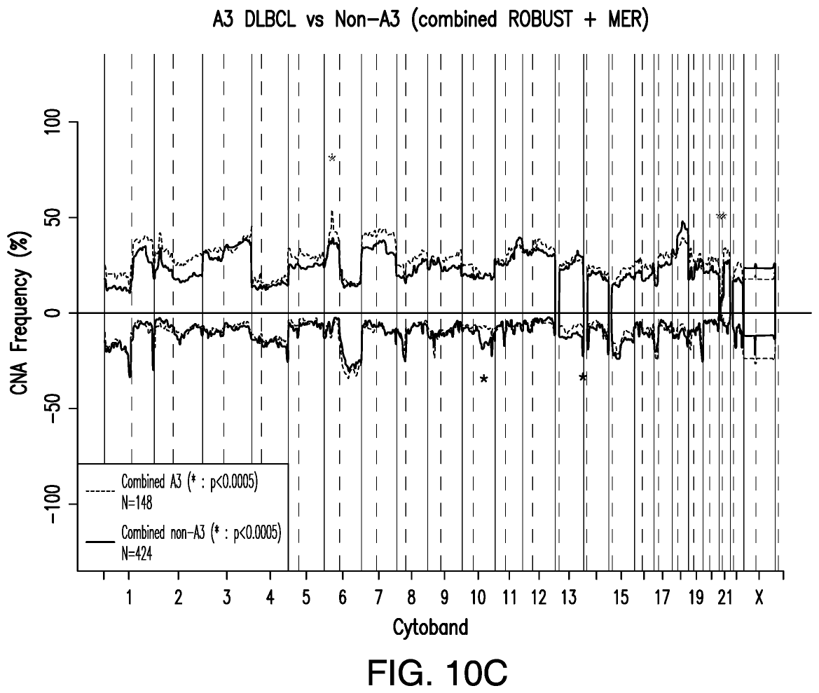

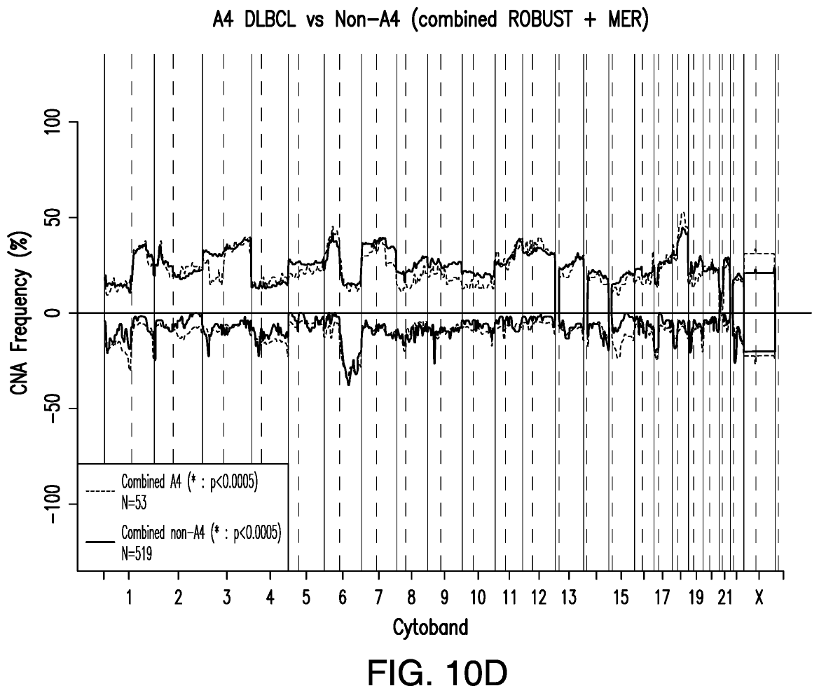

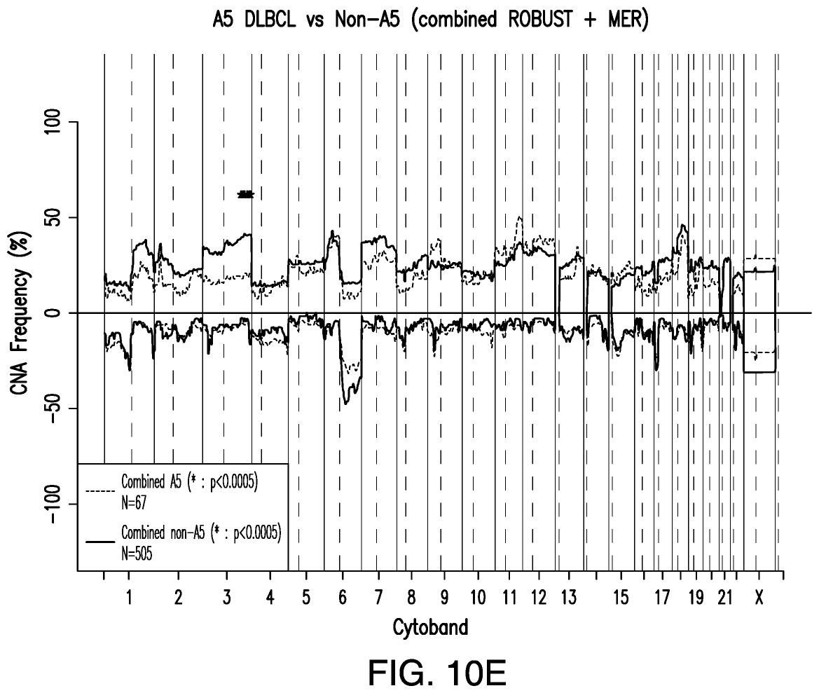

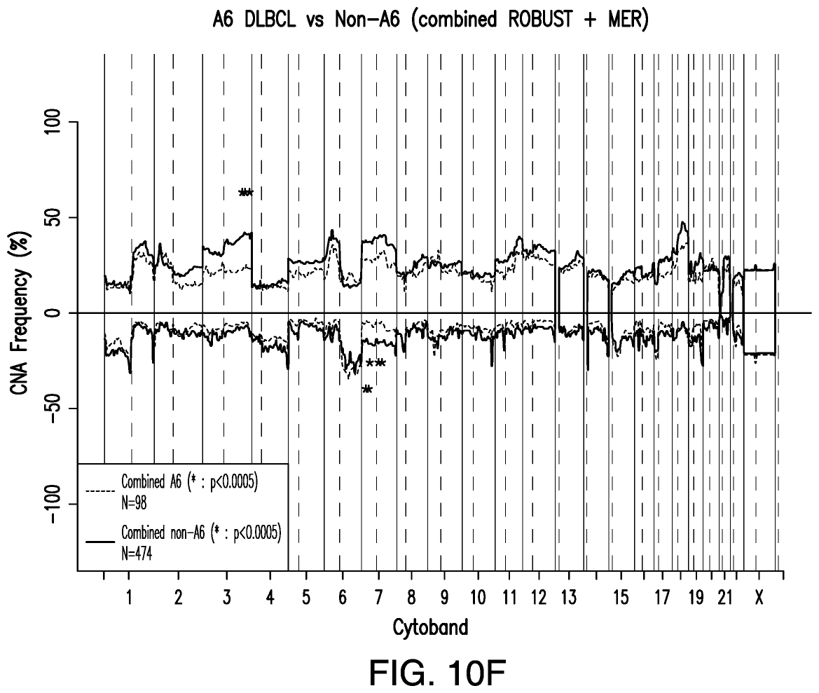

Genomic features enriched in A7 reflected the ABC-enriched nature of the cluster, with increased prevalence of mutations such as ETV6, PIM1, and OSBPL10 (FIG. 3C). In general, however, SNVs were not strongly associated with our clusters, which was not surprising as the clusters were derived from transcriptional features which could descend from sources other than SNVs such as copy number and epigenetic changes. Significantly enriched CNAs for each cluster were shown in FIG. 3D, with A7-associated features including arm-level copy number gains in chromosomes 3 and 18. CNA features of each cluster were shown in FIGS. 10A-10F.

Immunohistochemistry data validated the gene expression-derived patterns of immune infiltration across the clusters. Compared to non-A7, there was a consistent decrease of CD3, CD4 and CD8 T cells, and no strong trend for CD163 monocytes/macrophages, CD68 macrophage and CD11 dendritic cells (FIGS. 11A-11F). A representative MIBI image of the high-TME26 cluster A6, for example, indeed showed high abundance of CD4/CD8 T-cells, while the low-TME26 cluster A7 conversely showed a paucity of T-cells and an abundance of CD20 B-cells (FIG. 3E).

### 7.2.4 MYC Dysregulation was a Key Component to High-Risk Cluster A7 Biology and was Targetable Via TCF4

To further define biology specific to cluster A7, GSEA analysis was performed to identify pathways differentially expressed in A7. This cluster showed upregulation of MYC target signatures, E2F target signatures, and metabolism pathways such as G2M checkpoint and oxidative phosphorylation, and downregulation of immune and inflammatory signatures including TNFα, IL2, IL6, IFN-alpha, and IFN-gamma signaling pathways (FIG. 4A).

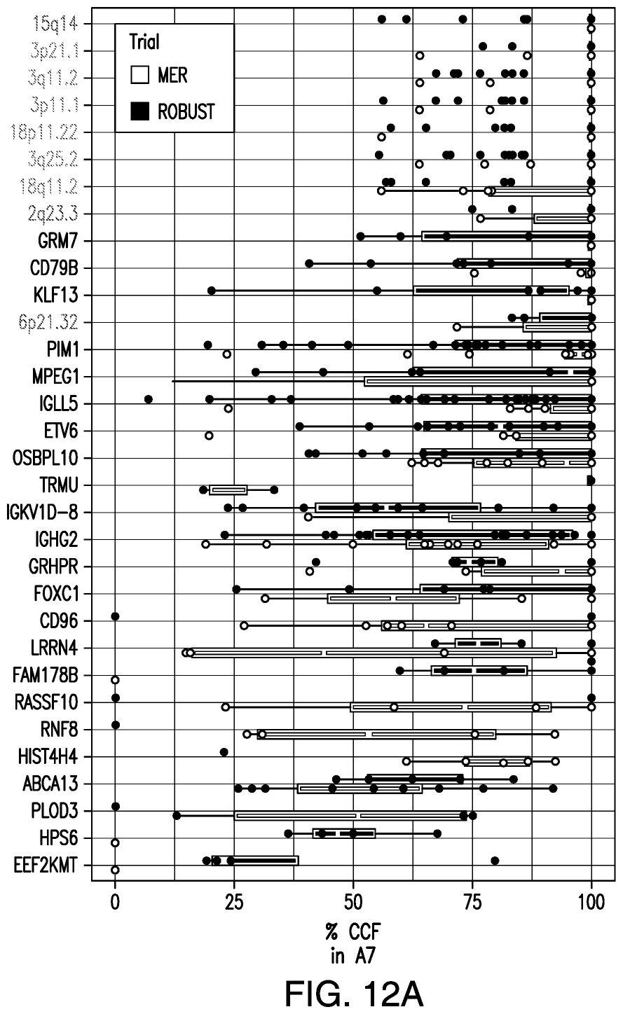

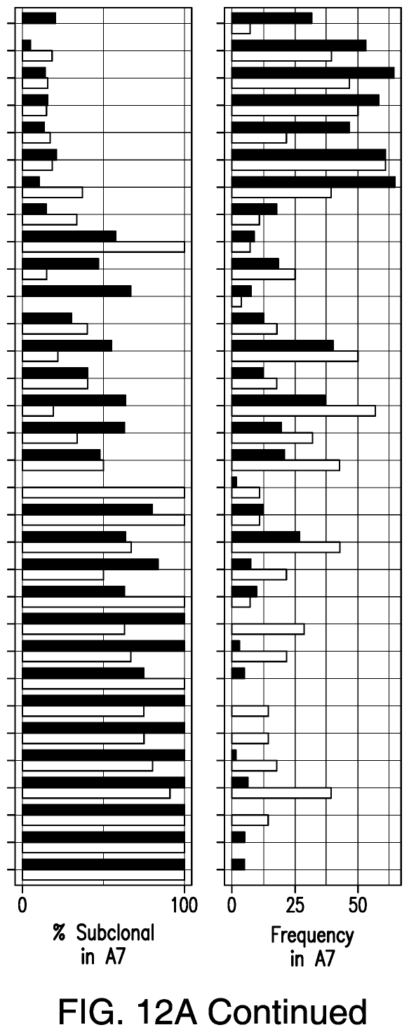

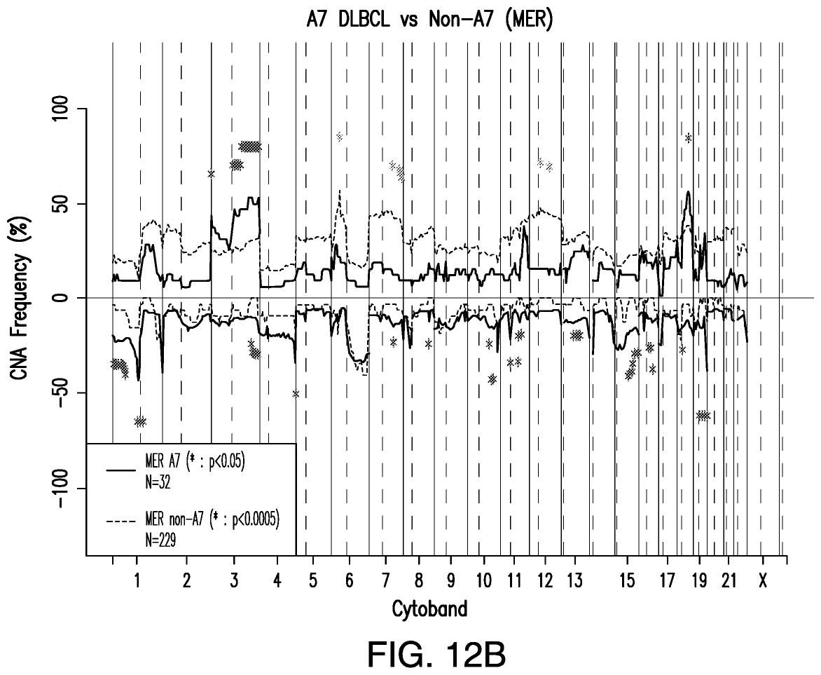

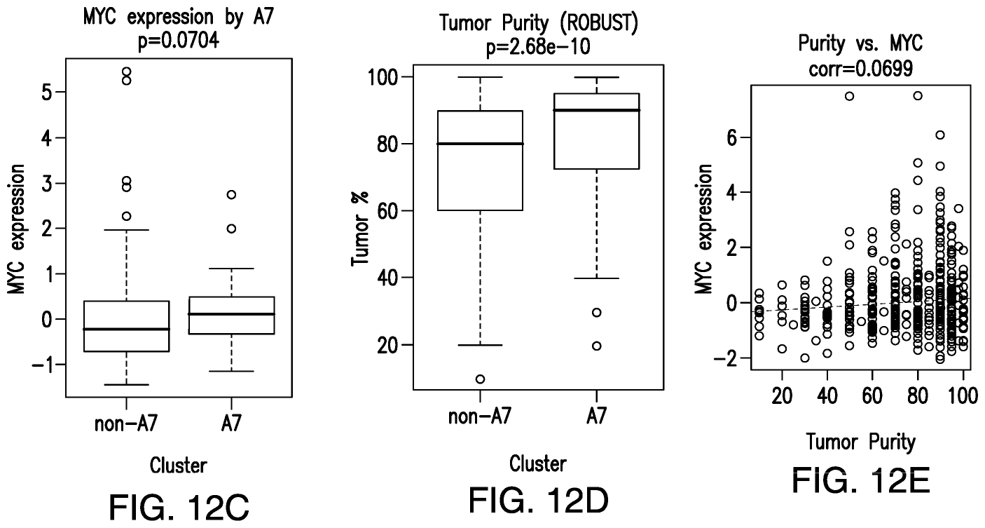

MYC gene expression was also upregulated in cluster A7 vs non-A7 (FIG. 4B), and was not driven by high tumor cellularity (FIG. 12E). Protein expression quantified by IHC also indicated that Myc protein levels were higher in A7 than non-A7 (FIG. 4C). Although MYC translocation and amplification well known to drive MYC signaling in B-cell lymphomas, neither MYC translocation nor MYC amplification was enriched in A7 (p=0.99), suggesting upregulated MYC activity was driven by other mechanisms.

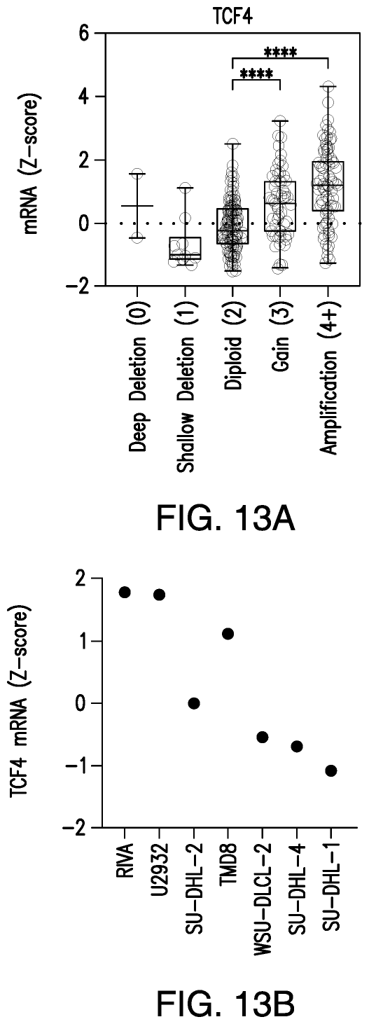

Significant enrichment of several arm-level amplifications in A7 was observed, with chromosome 18q and 3q amplification exhibiting the highest prevalence. Interestingly, 18q12.2 harbors the gene TCF4, which encoded a basic helix-loop-helix (bHLH) transcription factor reported to drive MYC gene expression by binding to its enhancer [PMID: 31217338]. A strong correlation was observed of TCF4 gene expression with TCF4 copy number in the Discovery and MER cohorts (FIGS. 13A-13B). It was then sought to characterize TCF4 functions using DLBCL cell line models. Knockdown of TCF4 dramatically reduced MYC protein expression in the TCF4 amplified cell lines (RIVA and U2932), but not those without TCF4 amplification (SU-DHL-2 and TMD8) (FIG. 4F), suggesting TCF4 amplification contributed to MYC over expression in ABC DLBCL. In line with these observations, knockdown of TCF4 strongly inhibited cell proliferation in TCF4-amplified cell lines (RIVA and U2932), whereas induction of the same shRNAs only modestly inhibited proliferation of cell lines without TCF4 amplification (SU-DHL-2 and TMD8) (FIG. 4G). Taken together, the amplification-dependent overexpression of TCF4 stimulated MYC expression and rendered ABC DLBCLs to be addictive to the overexpressed TCF4. TCF4 could be a potential therapeutic target for the A7 population.

### 7.2.5 Clinical Utility of Cluster A7

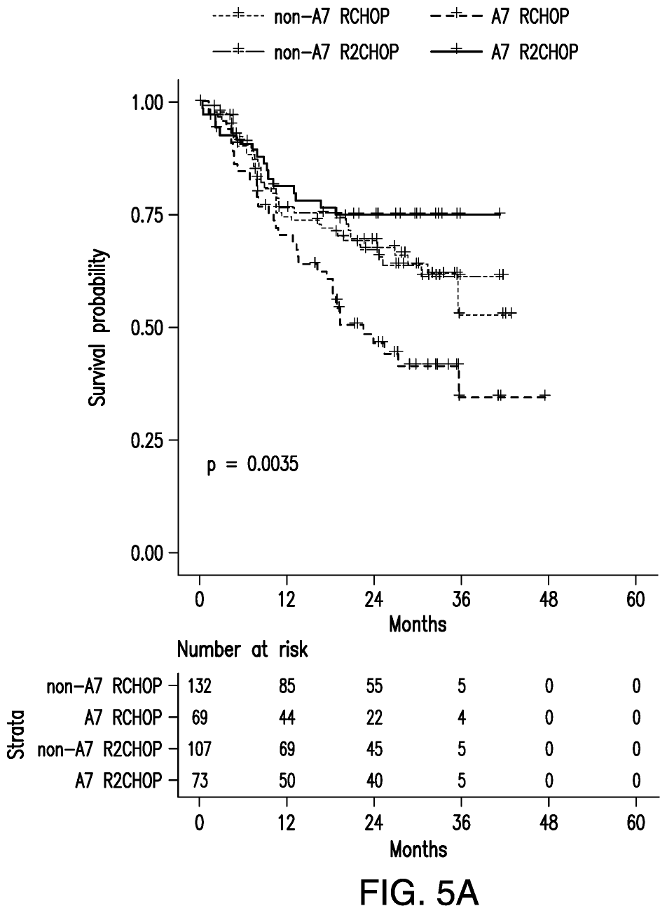

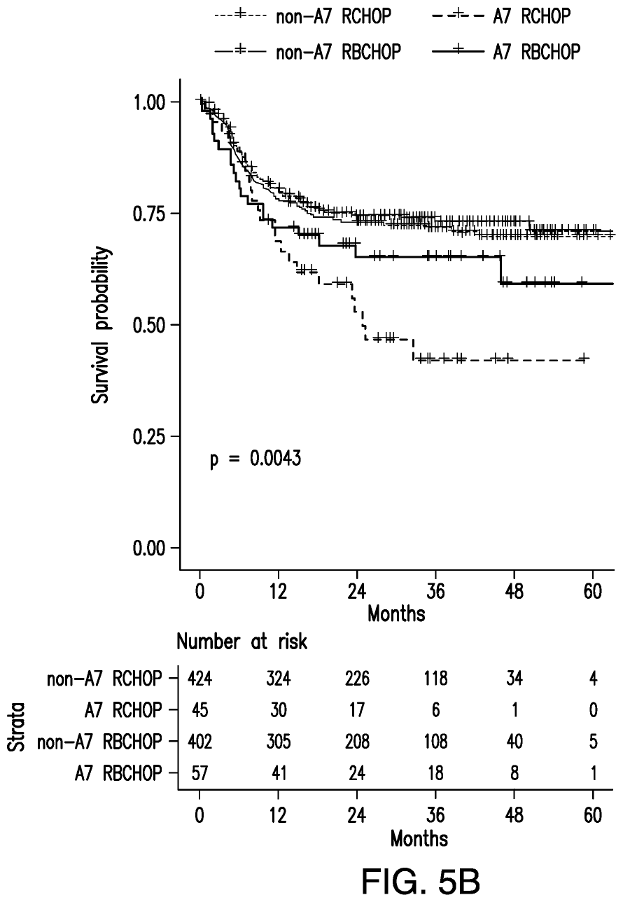

To assess the utility of A7 as a predictive patient population, ROBUST and REMoDL-B patients were retrospectively stratified by A7 versus non-A7 status in (FIGS. 5A-5B). The results indicated that both ROBUST and REMODL-B trials showed differences between the control arm and experimental arm in the A7 population (p=0.0088 and 0.16, respectively), indicating cluster A7 served as a more homogeneous and reliably high-risk patient population for drug development. With greater molecular and biological insights underpinning the tumor and TME of DLBCL beyond COO, the field was ripe for selection of high-risk patients, creative trial designs, and targeted therapies to shift clinical practice. Here, a high-risk patient segment in newly diagnosed DLBCL through unsupervised clustering of transcriptomic data was identified. Underneath the clinical high-risk behavior of A7 there were three biological features known to confer poor outcome: extreme ABC subtype, low immune infiltration (particularly low CD4 and CD8 T cells), and elevated MYC pathway.

Comparisons to recently published molecular classifications indicated cluster A7 had some unique features but was not mutually exclusive from others' clusters. The feature of depleted immune infiltration, a hallmark of A7, was shared with the “Depleted” segment (DP) (Kotlov, et al., 2021) and the Lymphoma Ecotype 1 (LE1) (Steen, et al., 2021), which exhibited similar unfavorable survival characteristics.

Cluster A7 also shared enrichment of previously defined features including amplifications on chromosomes 3p, 3q and 18q and mutations in PIM1, ETV6 and OSBPL10, with genetic subtypes C5 and MCD (based on the co-occurrence of MYD88^{L265P} and CD79B mutations as described in Schmitz et al., 2018, New England Journal of Medicine, 378(15), 1396-1407). The co-occurrence of the MCD and A7 clusters was investigated in the NCI dataset (Schmitz et al., 2018, New England Journal of Medicine, 378(15), 1396-1407), for which the LymphGen calls were publicly available. Roughly ⅓ of patients identified as either A7 or MCD were also identified as the other, a statistically significant overlap (p=0.038). Even though A7 and MCD both identified patient groups of similar size and risk, the majority of cases in each cluster represented a unique subset of high-risk patients not identified by the other method (FIGS. 14A-14B).

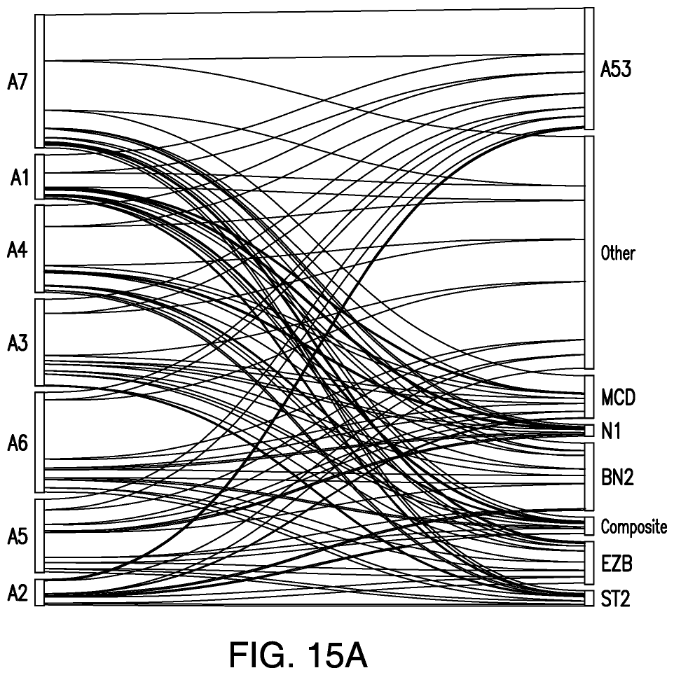

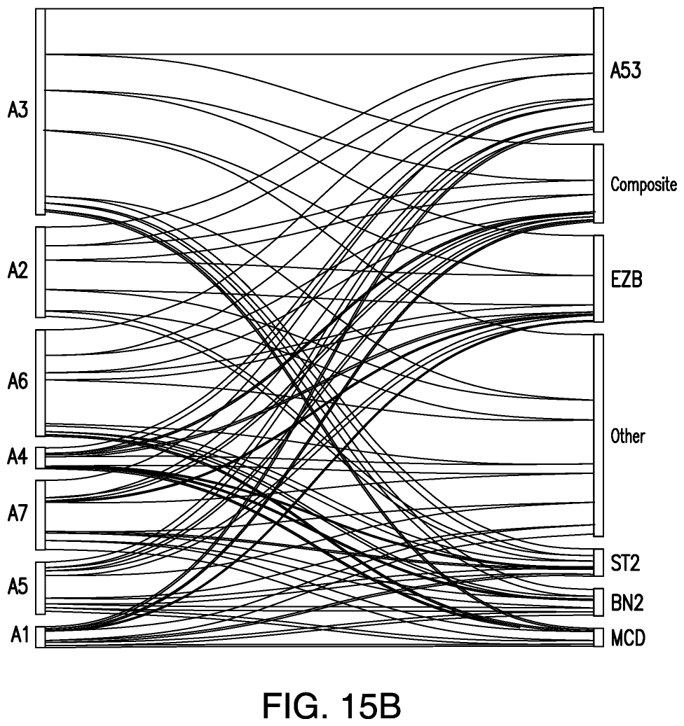

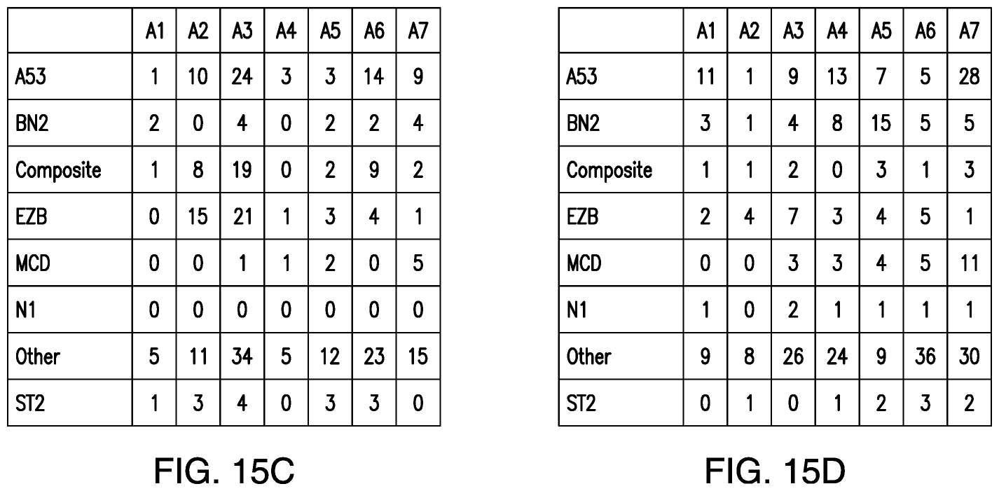

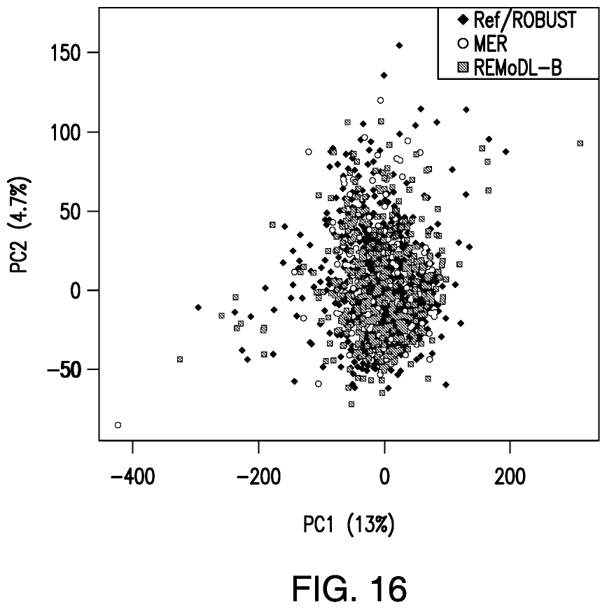

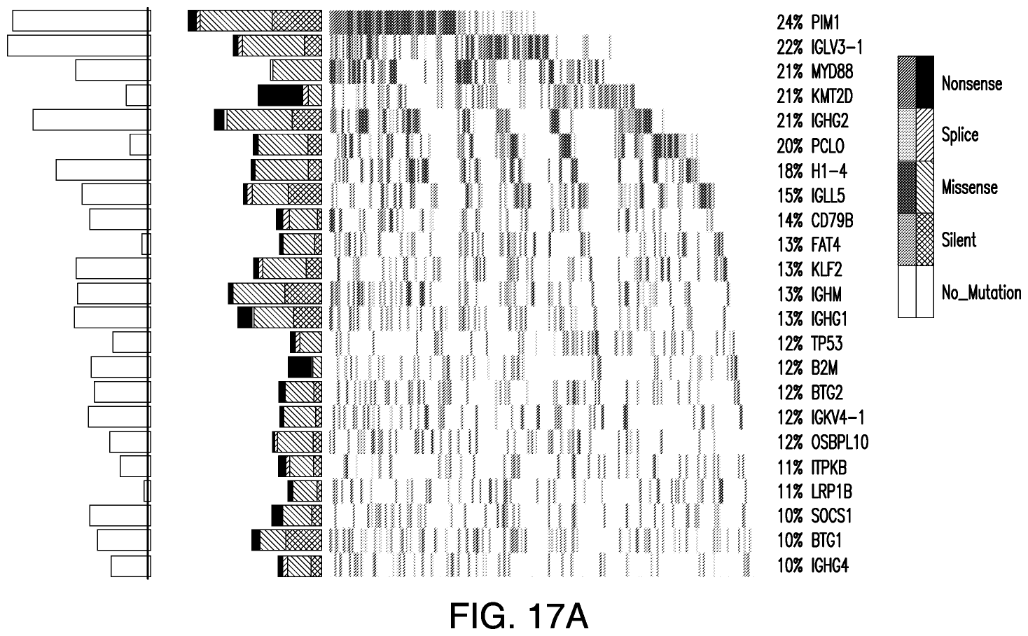

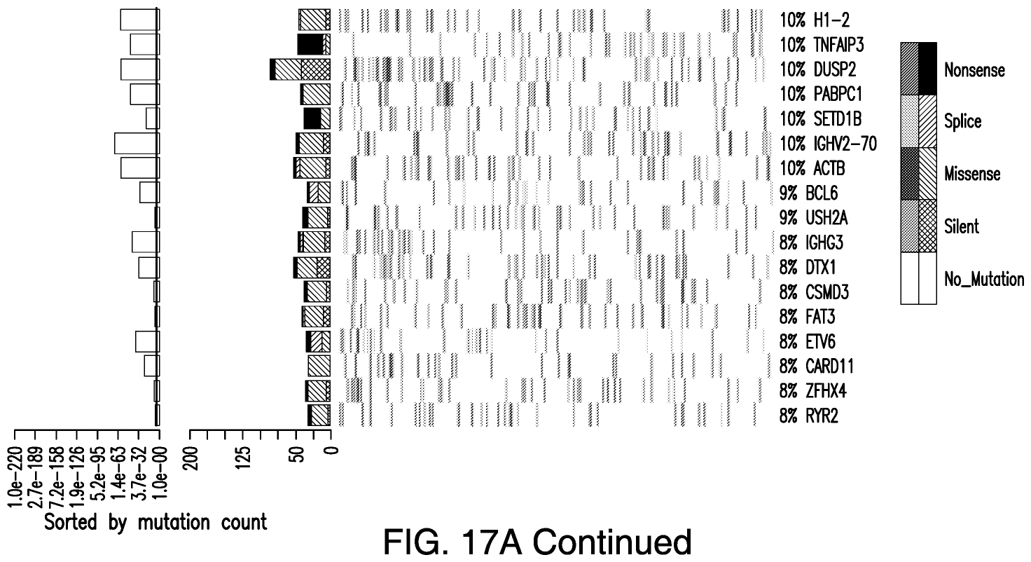

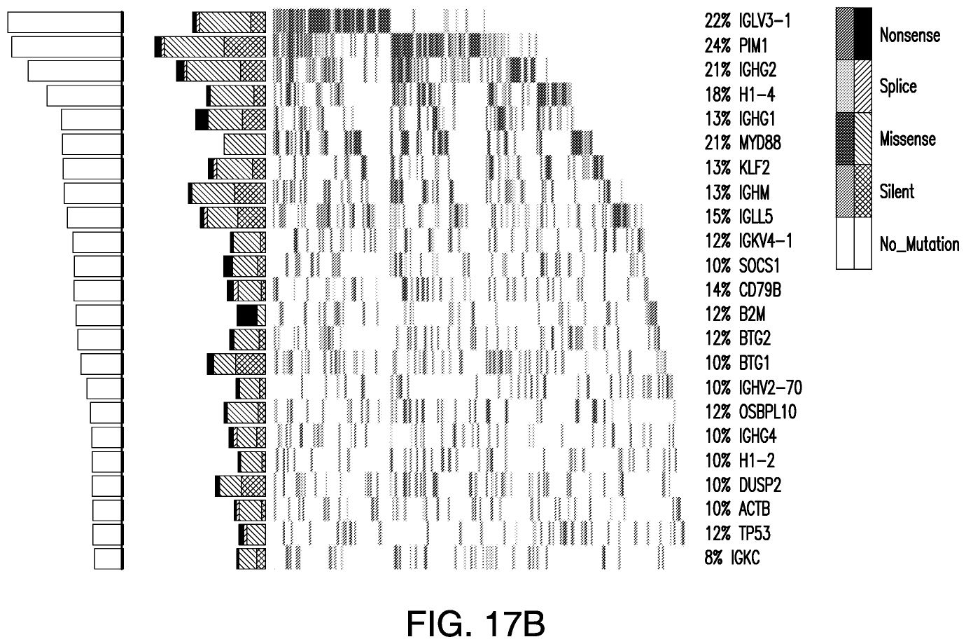

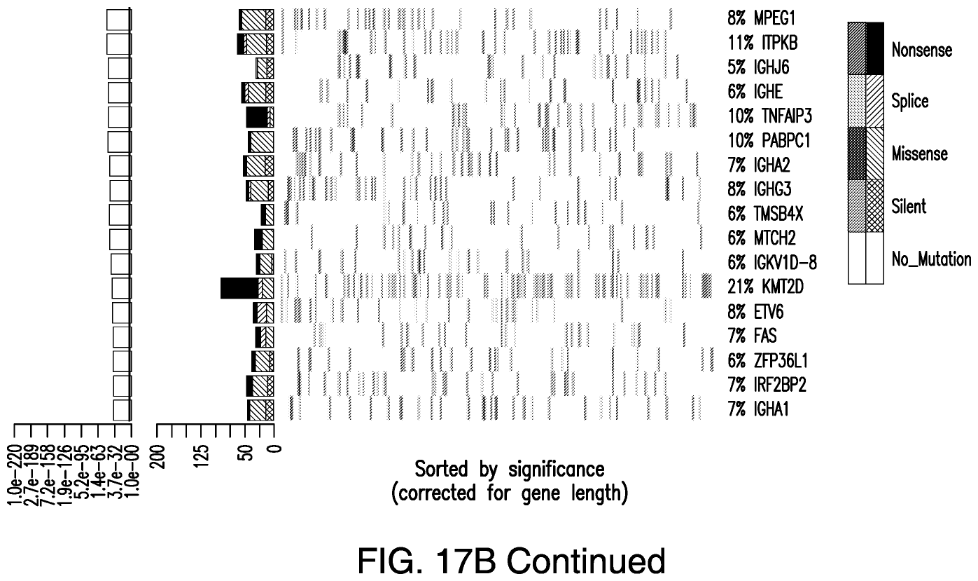

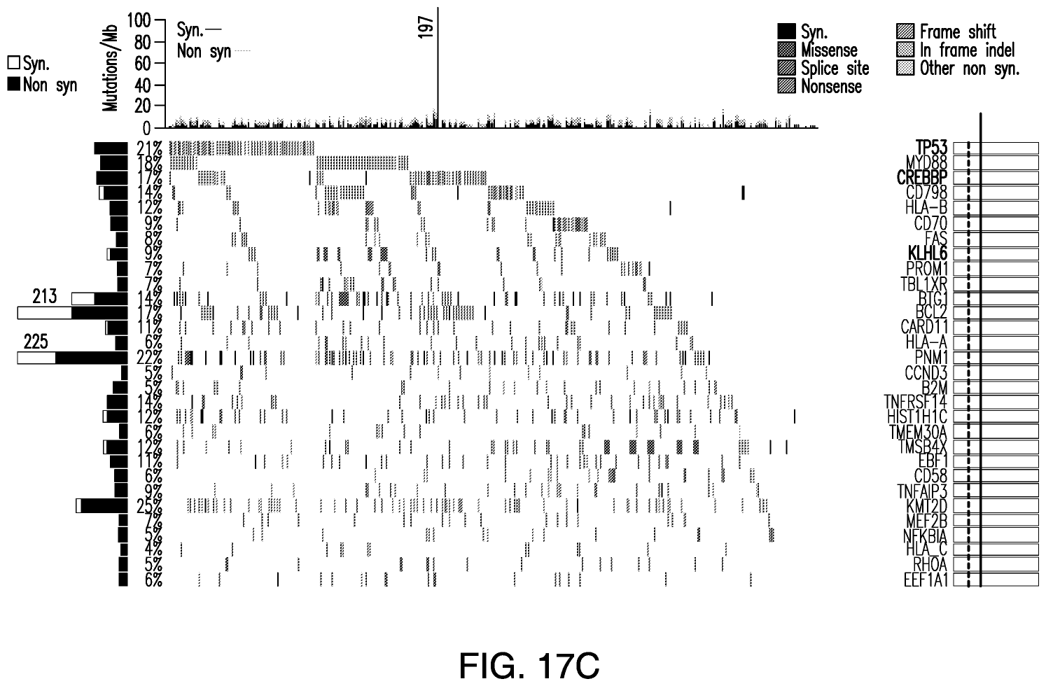

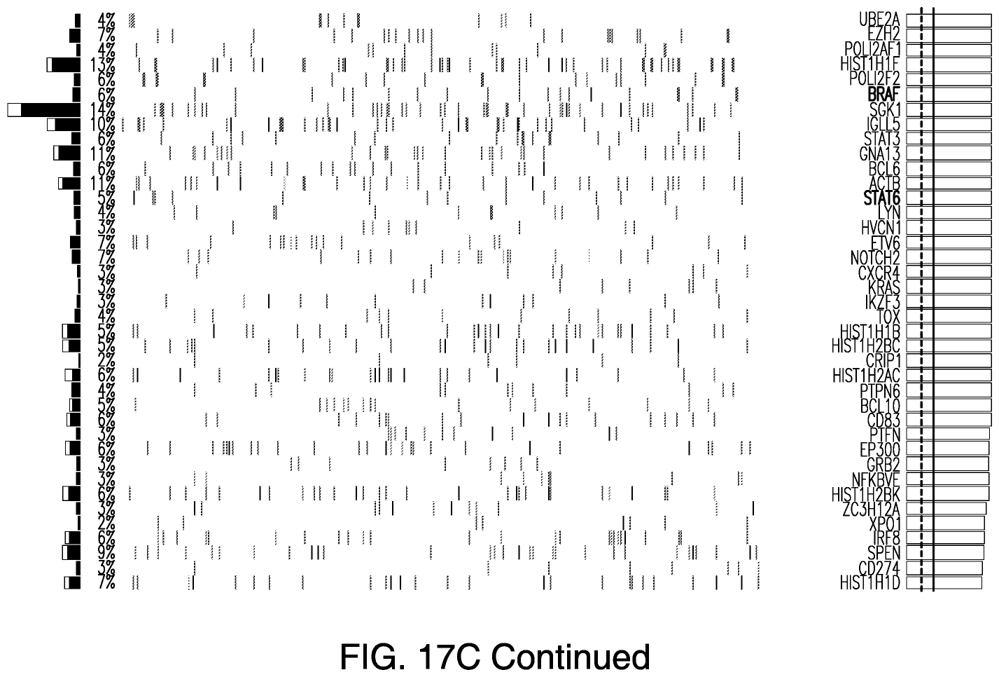

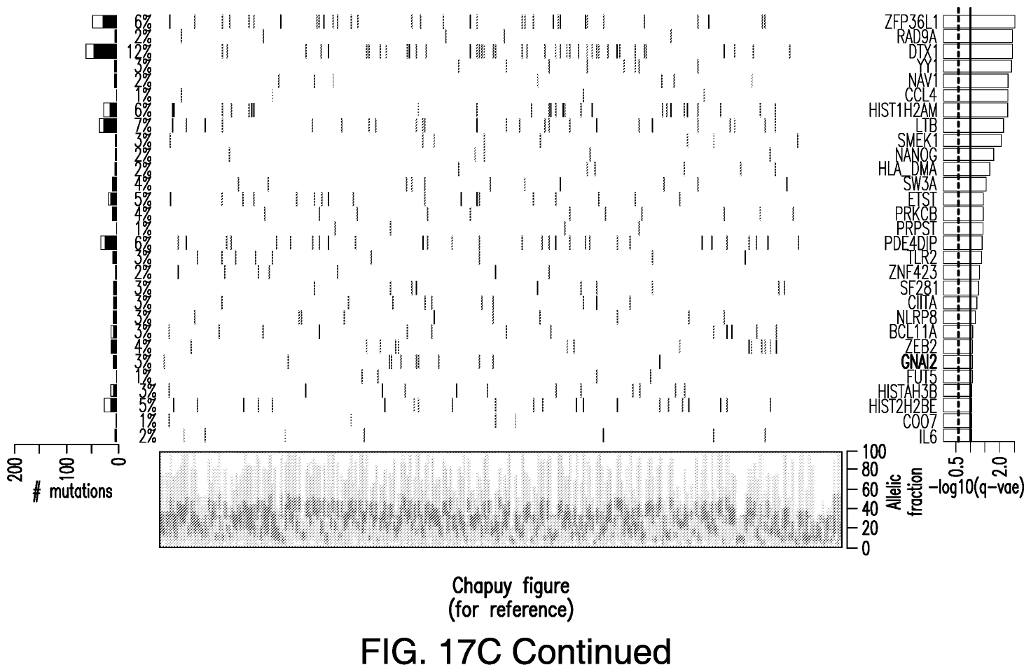

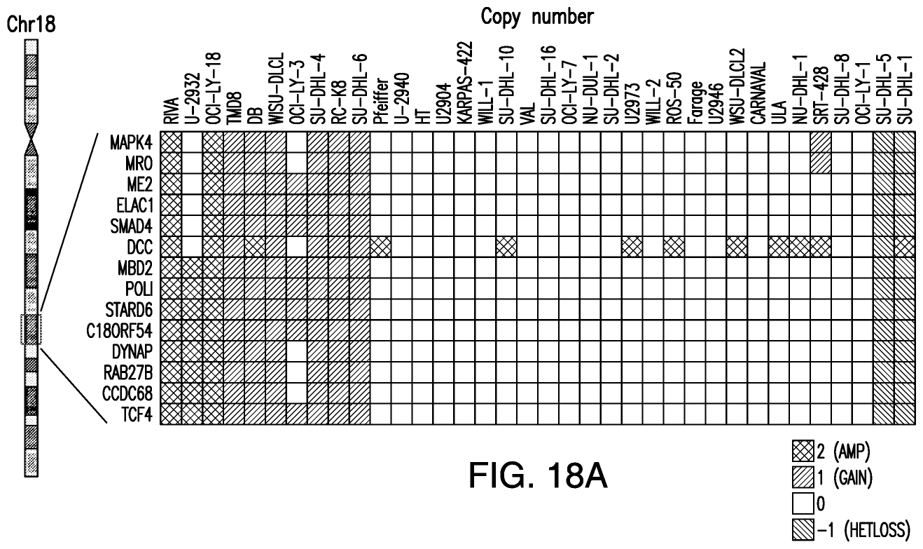

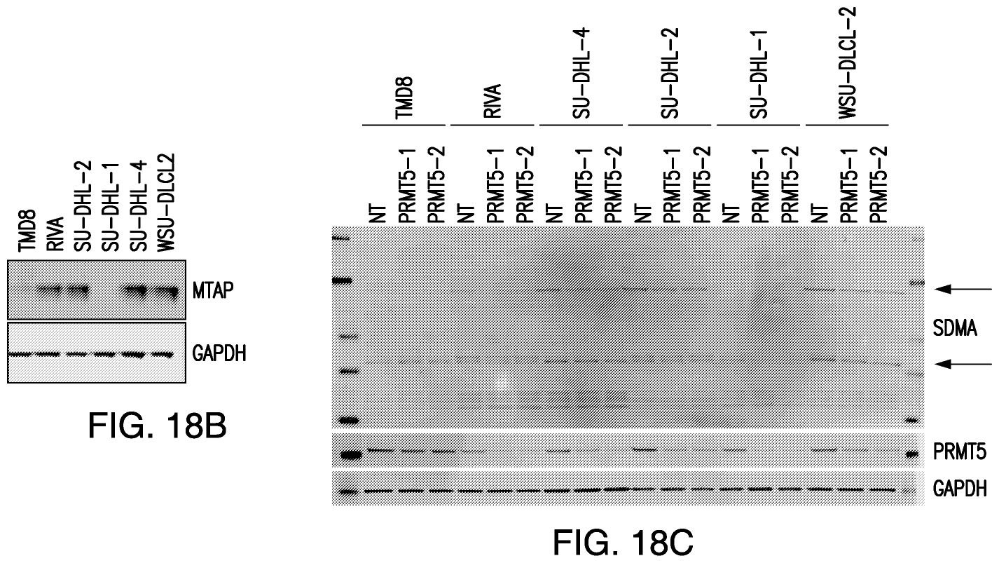

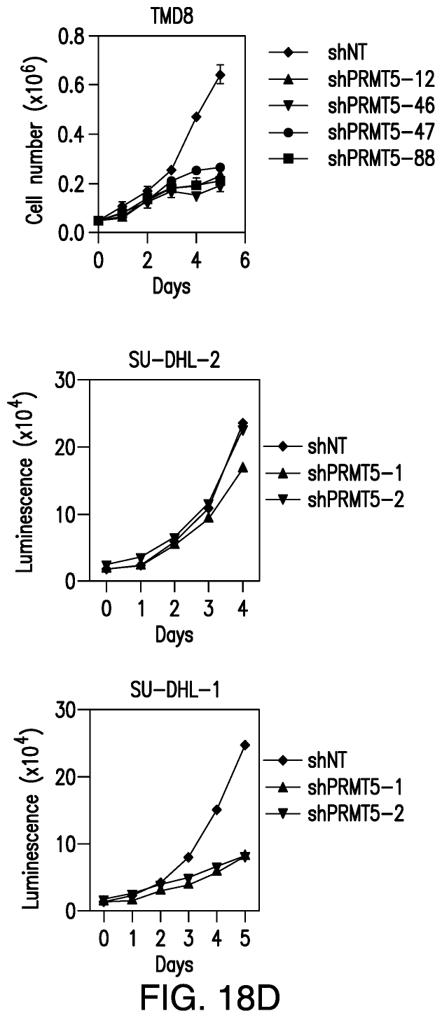

The MCD subtype (based on the co-occurrence of MYD88^{L265P} and CD79B mutations as described in Schmitz et al., 2018, New England Journal of Medicine, 378(15), 1396-1407) was enriched for A7 patients, while the EZB subtype (based on EZH2 mutations and BCL2 translocations as described in Schmitz et al., 2018, New England Journal of Medicine, 378(15), 1396-1407) was enriched for GCB-like clusters A2 and A3 (FIGS. 15A-15D). Although a statistically significant association existed between the classification methods (Fisher p=0.0005 in ROBUST, p=0.001 in MER), there was a great deal of heterogeneity and no clear one-to-one mapping between any of the subtypes. In addition, PCA plot of the Discovery, MER, and REMoDL-B datasets after normalization showed no dataset-specific differences (FIG. 16). Mutation landscape (Chapuy genes), which is sorted by: mutation count (FIG. 17A), by significance (corrected for gene length) (FIG. 17B), and Chapuy figure (for reference) (FIG. 17C) was also measured. The expression of proteins encoded by genes of chromosome 18 was also accessed (FIG. 18A-18D).

An elevated MYC pathway has been associated with poor survival in DLBCL (Savage, et al., 2009, MYC gene rearrangements are associated with a poor prognosis in diffuse large B-cell lymphoma patients treated with R-CHOP chemotherapy. Blood, 114(17), 3533-3537) (Barrans, et al., 2010, Rearrangement of MYC is associated with poor prognosis in patients with diffuse large B-cell lymphoma treated in the era of rituximab. Journal of clinical oncology, 28(20), 3360-3365), though the mechanisms are different for GCB and ABC subtypes. In GCB, chromosomal rearrangement of MYC and BCL2 to the IG locus is the main driver behind MYC and BCL2 overexpression. In ABC tumors, MYC translocations are relatively rare, and MYC over-expression is not associated with translocation events (Xu-Monette, et al., 2015, Clinical features, tumor biology, and prognosis associated with MYC rearrangement and Myc overexpression in diffuse large B-cell lymphoma patients treated with rituximab-CHOP. Modern Pathology, 28(12), 1555-1573). It has also been shown that MYC expression is not affected by its copy number gain (Collinge, et al., 2021, The impact of MYC and BCL2 structural variants in tumors of DLBCL morphology and mechanisms of false-negative MYC IHC. Blood, 137(16), 2196-2208). Similar patterns in A7 were found, with no difference in MYC translocation or copy number gain between A7 and non-A7 cases, yet both gene and protein expression of MYC were elevated in A7. The notion that certain MYC regulators such as TCF4, which was amplified as part of 18q gain, were responsible for this increase was tested. The data in ABC cell lines demonstrated this linkage and indicated that TCF4 served as a therapeutic target for A7 (FIGS. 4E-4G). Other MYC regulators could share similar functional impact.

Additional pathway changes unique to A7 included upregulation of G2M checkpoint, mitotic spindle checkpoint and DNA repair pathways (FIG. 4A), indicating cell cycle deregulation and stress of DNA replication. Coupled with down-regulation of the p53 pathway these changes were likely to result in rapid proliferation and genomic instability which were supported by uncontrolled growth and many copy number alterations (FIG. 3D). Another important feature of A7 was upregulation of the oxidative phosphorylation pathway, indicating altered energy metabolism by the tumor through utilizing oxidizable substrates such as fatty acid in low oxygen microenvironments. Both phenomena had been reported as a molecule hallmarks for subsets of DLBCL (Monti, et al., 2012, Molecular profiling of diffuse large B-cell lymphoma identified robust subtypes including one characterized by host inflammatory response. Blood, 105(5), 1851-1861), (Caro, et al., 2012, Metabolic signatures uncover distinct targets in molecular subsets of diffuse large B cell lymphoma. Cancer Cell, 22(4), 547-560). These observations led to novel treatment strategies and targets for this high-risk segment.

Indeed, the poor prognosis of A7 demanded a different approach than R-CHOP. Here, an example was shown of the R2-CHOP regimen that significantly improved the outcome of A7 patients, even though R2-CHOP was not specifically designed for this population (FIG. 5A). Lenalidomide is a cereblon-modulating agent that has dual effects—an autonomous antiproliferative on tumor B-cell and an immune-mediated cytotoxicity (Garciaz, et al., 2016, Lenalidomide for the treatment of B-cell lymphoma. Expert opinion on investigational drugs, 25(9), 1103-1116). The immunomodulation activity was particularly beneficial to “cold tumors” such as those typified by A7. Ibrutinib also performed well in the A7 cluster suggesting the extreme ABC biology of A7 interacted with BCR modulating agents. These examples demonstrated the utility of the A7 gene classifier tool for use in patient selection and potential clinical value of this high-risk patient segment.

Finally, the biomarkers used to identify A7 patients had several attributes which were attractive from a practical implementation perspective. First, gene expression tests were easy to administer with proven feasibility in the clinical trial setting, as seen in ROBUST with a 2.4 day turn-around time. Second, it used diagnostic FFPE tissue and did not require an additional biopsy. Third, it classified all patients either A7 or not without the ambiguity of an unclassifiable population. The future of DLBCL, similar to the paradigm shift which occurred in AML with FLT3 and IDH2 inhibitors, was targeted treatment of molecularly defined patient segments using practical assays for decisions of risk and treatment. This work along with the recent wave of DLBCL classification tools was a major advance in that direction.

TABLE 2
Demographics and clinical characteristics of the discovery and validation cohorts.

| | ndMER- | | REMoDL-B | | (R-ROBUST- | | ROBUST- | |
|---|---|---|---|---|---|---|---|---|
| | RNAseq | | CHOP) | | RNAseq ITT | | RNAseq | |
| | (N = 343) | | (N = 469) | | (n = 392) | | (n = 1016) | |
| Sex | | | | | | | | |
| Female | 144 | (0.42) | 205 | (0.44) | 180 | (0.46) | 484 | (0.48) |
| Male | 199 | (0.58) | 264 | (0.56) | 212 | (0.54) | 532 | (0.52) |
| Age (year) | | | | | | | | |
| Median | 65 | (18-90) | 66 | (24-86) | 66 | (21-83) | 65 | (18-86) |
| <60 | 124 | (0.36) | 150 | (0.32) | 117 | (0.3) | 378 | (0.37) |
| ≥60 | 219 | (0.64) | 319 | (0.68) | 275 | (0.7) | 638 | (0.63) |
| Ann Arbor | | | | | | | | |
| Stage | | | | | | | | |
| I-II | 146 | (0.43) | 146 | (0.31) | 41 | (0.1) | 217 | (0.21) |
| III-IV | 196 | (0.57) | 321 | (0.68) | 351 | (0.9) | 776 | (0.76) |
| ECOG PS | | | | | | | | |
| 0-1 | 281 | (0.82) | 417 | (0.89) | 329 | (0.84) | 329 | (0.32) |
| 2-4 | 61 | (0.18) | 52 | (0.11) | 63 | (0.16) | 63 | (0.06) |
| Elevated | 164 | (0.48) | 363 | (0.77) | 230 | (0.59) | 230 | (0.23) |
| LDH | | | | | | | | |
| Extranidal | | | na | | | | | |
| site | | | | | | | | |
| 0-1 | 278 | (0.81) | | | 224 | (0.57) | 224 | (0.22) |
| >1 | 65 | (0.19) | | | 168 | (0.43) | 168 | (0.17) |
| IPI Group | | | | | | | | |
| Low (0-2) | 215 | (0.63) | 239 | (0.51) | 182 | (0.46) | 182 | (0.18) |
| High (3-5) | 128 | (0.37) | 230 | (0.49) | 208 | (0.53) | 258 | (0.25) |
| COO | | | | | | | | |
| ABC | 155 | (0.45) | 192 | (0.41) | 352 | (0.9) | 505 | (0.5) |
| GCB | 152 | (0.44) | 218 | (0.46) | 5 | (0.01) | 352 | (0.35) |
| UNC | 36 | (0.1) | 59 | (0.13) | 35 | (0.09) | 159 | (0.16) |
| TME | | | | | | | | |
| Positive | 145 | (0.42) | | | 98 | (0.25) | 360 | (0.35) |
| Negative | 198 | (0.58) | | | 294 | (0.75) | 656 | (0.65) |
| DHITSig | | | | | | | | |
| Positive | 54 | (0.16) | | | 16 | (0.04) | 142 | (0.14) |
| Negative | 289 | (0.84) | | | 376 | (0.96) | 871 | (0.86) |
| LGP cluster | | | | | | | | |
| A7 | 39 | (0.11) | 42 | (0.09) | 144 | (0.37) | 203 | (0.2) |
| A7 in ABC | 34 | (0.21) | 36 | (0.19) | 139 | (0.39) | 174 | (0.34) |
| Med PFS A7 | NA | | 25.3 | | 29.7 | | — | |
| (months) | | | | | | | | |
| Med PFS non- | NA | | Not achieved (NA) | | 27.0 | | — | |
| A7 (months) | | | | | | | | |
| Med OS A7 | NA (doesn't | | Not achieved (NA) | | 30.4 | | — | |
| (months) | reach 50% | | | | | | | |
| | survival) | | | | | | | |
| Med OS non- | 165.6 | | Not achieved (NA) | | 27.3 | | — | |
| A7 (months) | | | | | | | | |
| % CR in A7 | 27 | (0.69) | 26 | (0.62) | 100 | (0.69) | — | |
| % EFS24 in A7 | 19 | (0.49) | | | | | | |

### 7.3 Example 3: Classified Subgroups A1 Through A8

Integrative clustering identified eight subgroups of ndDLBCL patients (named A1-A8). The resulting clusters were analyzed in the lens of different biological features including gene signatures such as Double HIT gene (DHIT+) signature and TMD gen signature (TME+). The prevalence and biological features (such as COO type, DHITsig positivity, TME gene signature positivity, BCL2/BCL6 translocation, and MYC translocation) of the replication dataset (MER dataset) were summarized in Table 3. The resulting cluster identification were predictive of the likelihood of response to standard treatment (e.g., R-CHOP combination treatment) and suggested rational targeted therapies based on cluster-specific biological features.

TABLE 3
Prevalence and biological features of replication dataset (MER dataset)

| | | | | % | % | | % | |
|---|---|---|---|---|---|---|---|---|
| | | | | BCL2 | BCL6 | % MYC | MYC | % |
| Cluster/ | Prevalence | % | % | FISH | FISH | FISH | FISH | DHIT |
| Subgroup | in nd | GCB | TME+ | transloc. | transloc. | transloc. | DHIT | sig+ |
| 1 | 4% | 60% | 13% | 27% | 20% | 20% | 9% | 33% |
| 2 | 17% | 87% | 17% | 35% | 17% | 8% | 7% | 30% |
| 3 | 31% | 53% | 48% | 21% | 7% | 14% | 14% | 24% |
| 4 | 3% | 0% | 36% | 0% | 18% | 9% | 0% | 18% |
| 5 | 9% | 25% | 34% | 9% | 34% | 9% | 7% | 3% |
| 6 | 23% | 33% | 94% | 16% | 15% | 5% | 3% | 4% |
| 7 | 12% | 3% | 5% | 5% | 30% | 8% | 3% | 10% |
| 8 | 0% | — | — | — | — | — | — | — |

This clustering method allowed for the transcriptomic identification of eight patients subgroups (e.g., subgroups A1 through A8). Among the identified patient groups, for example, subgroup A7 was a high-risk subgroup, which was underserved by the standard R-CHOP therapy.

Patients in the high-risk subgroup A7 showed i) low expression of TME signatures, including low level of infiltrating immune cells; ii) high expression of malignant processes such as MYC targets and proliferation; iii) high expression of tumor metabolism signatures (e.g., ribosome process and oxidative phosphorylation); iv) a mixture of B cell linage signatures; and v) upregulation of B cell transcription factors such as IRF4 and OCT-2.

### 7.4 Example 4: Cell Proportions in Subgroups

The total cell counts of different T-cells in the DLBCL patients in a subset of the discovery (Discovery-2) dataset (n=46) were measured using Multiplexed ion beam imaging (MIBI). The patients were from the identified subgroups A7 and non-A7. FIGS. 11A-11F illustrate the total cell counts in patients from different subgroups.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

What is claimed is:

1. A method for predicting the responsiveness of a lymphoma patient to a cancer treatment comprising:
(a) clustering reference lymphoma patients in a reference patient group into subgroups using the expression level of at least one gene in reference biological samples of the reference lymphoma patients;
(b) determining a subgroup to which the lymphoma patient belongs based on the expression level of the at least one gene in a biological sample from the lymphoma patient; and
(c) predicting the responsiveness of the lymphoma patient to a first cancer treatment based on the subgroup of the lymphoma patient.

2. The method of claim 1, further comprising administering to the lymphoma patient a second cancer treatment.

3. The method of claim 1 or 2, wherein step (a) comprises generating clustering information defining relationships between the expression level of the at least one gene in the reference biological samples, and rearranging a heat map representation based on the clustering information.

4. The method of any one of claims 1-3, wherein step (a) uses a hierarchical method or a non-hierarchical method.

5. The method of any one of claims 1-3, wherein step (a) uses iClusterPlus method.

6. The method of any one of claims 1-5, wherein the reference lymphoma patients are clustered into 2-12 subgroups.

7. The method of claim 6, wherein the reference lymphoma patients are clustered into 7 subgroups.

8. The method of any one of claims 1-7, wherein the method further comprises training a classifier model using the expression level of the at least one gene in the reference biological samples.

9. The method of anyone of claims 1-8, wherein the at least one gene is selected from the genes of Table 1, optionally wherein the at least one gene comprises five or more genes of Table 1.

10. The method of claim 9, wherein the at least one gene comprises all genes of Table 1.

11. The method of any one of claims 8-10, wherein the classifier model is a grouped multinomial generalized linear model (GLM).

12. The method of any one of claims 8-11, wherein the classifier model is a binary model.

13. The method of any one of claims 8-12, wherein the method further comprises setting a threshold confidence level for at least one of the subgroups of step (a) to exclude patients that give lower confidence level clustering data from the at least one subgroup.

14. The method of any one of claims 1-13, wherein the lymphoma is selected from the group consisting of diffuse large B-cell lymphoma (DLBCL), indolent B cell lymphoma, follicular lymphoma, small lymphocytic lymphoma, nodal marginal zone B-cell lymphoma, lymphoplasmacytic lymphoma, anaplastic large cell lymphoma, primary cutaneous type lymphoma, mycosis fungoides, chronic lymphocytic leukemia, and mantle cell Lymphoma.

15. The method of 14, wherein the lymphoma is DLBCL.

16. The method of claim 14, wherein the lymphoma is indolent B cell lymphoma, nodal marginal zone B-cell lymphoma, mantle cell lymphoma, or chronic lymphocytic leukemia.

17. The method of any one of claims 1-16, wherein the reference patients in the reference patient group are clustered into subgroups A1-A7, and wherein:
(i) subgroup A1 comprises about 50% to about 60% patients having germinal center B-cell-like (GCB) DLBCL, about 30% to about 40% patients having activated B-cell like (ABC) DLBCL, about 10% to about 20% patients who are TME+ DLBCL patients, and about 30% to about 40% patients who are DHITsig+ DLBCL patients;
(ii) subgroup A2 comprises about 80% to about 90% patients having GCB DLBCL, about 0% to about 5% patients having ABC DLBCL, about 15% to about 25% patients who are TME+ DLBCL patients, and about 25% to about 35% patients who are DHITsig+ DLBCL patients;
(iii) subgroup A3 comprises about 40% to about 55% patients having GCB DLBCL, about 30% to about 45% patients having ABC DLBCL, about 40% to about 50% patients who are TME+ DLBCL patients, and about 20% to about 30% patients who are DHITsig+ DLBCL patients;
(iv) subgroup A4 comprises about 25% to about 35% patients having GCB DLBCL, about 40% to about 50% patients having ABC DLBCL, about 30% to about 40% patients who are TME+ DLBCL patients, and about 10% to about 20% patients who are DHITsig+ DLBCL patients;
(v) subgroup A5 comprises about 20% to about 40% patients having GCB DLBCL, about 45% to about 65% patients having ABC DLBCL, about 30% to about 40% patients who are TME+ DLBCL patients, and about 0% to about 10% patients who are DHITsig+ DLBCL patients;
(vi) subgroup A6 comprises about 30% to about 40% patients having GCB DLBCL, about 40% to about 50% patients having ABC DLBCL, about 75% to about 95% patients who are TME+ DLBCL patients, and about 0% to about 10% patients who are DHITsig+ DLBCL patients; and
(vii) subgroup A7 comprises about 0% to about 10% patients having GCB DLBCL, about 80% to about 90% patients having ABC DLBCL, about 0% to about 10% patients who are TME+ DLBCL patients, and about 0% to about 15% patients who are DHITsig+ DLBCL patients.

18. The method of any one of claims 1-17, wherein the first cancer treatment is a combination treatment with rituximab, cyclophosphamide, doxorubicin, vincristine, and prednisone (R-CHOP).

19. The method of any one of claims 1-18, wherein when the lymphoma patient is determined to belong to subgroup A1, the method comprises predicting that the patient is not likely to be responsive to the first cancer treatment.

20. The method of any one of claims 1-18, wherein when the lymphoma patient is determined to belong to subgroup A2, the method comprises predicting that the patient is not likely to be responsive to the first cancer treatment.

21. The method of any one of claims 1-18, wherein when the lymphoma patient is determined to belong to subgroup A3, the method comprises predicting that the patient is not likely to be responsive to the first cancer treatment.

22. The method of any one of claims 1-18, wherein when the lymphoma patient is determined to belong to subgroup A4, the method comprises predicting that the patient is not likely to be responsive to the first cancer treatment.

23. The method of any one of claims 1-18, wherein when the lymphoma patient is determined to belong to subgroup A5, the method comprises predicting that the patient is not likely to be responsive to the first cancer treatment.

24. The method of any one of claims 1-18, wherein when the lymphoma patient is determined to belong to subgroup A6, the method comprises predicting that the patient is not likely to be responsive to the first cancer treatment.

25. The method of any one of claims 1-18, wherein when the lymphoma patient is determined to belong to subgroup A7, the method comprises predicting that the patient is not likely to be responsive to the first cancer treatment.

26. The method of any one of claims 2-25, wherein the second cancer treatment is R-CHOP.

27. The method of any one of claims 2-25, wherein the second cancer treatment is not R-CHOP.

28. The method of claim 27, wherein the second cancer treatment is a bromodomain and extra-terminal (BET) inhibitor, or a cyclin dependent kinase (CDK) inhibitor.

29. A method for predicting the responsiveness of a lymphoma patient to a cancer treatment comprising:
(a) determining the expression level of at least one gene of Table 1 in a biological sample from a lymphoma patient, optionally wherein the at least one gene comprises five or more genes of Table 1;
(b) comparing the expression level of the at least one gene of step (a) with the expression level of the at least one gene in a reference biological sample from a reference lymphoma patient, wherein the reference lymphoma patient is responsive to the cancer treatment, and
wherein if the expression level of the at least one gene in the biological sample is similar to the expression level of the at least one gene in the reference biological sample, it indicates that the lymphoma patient is not likely to be responsive to the cancer treatment.

30. A method for predicting the responsiveness of a lymphoma patient to a cancer treatment comprising:
(a) determining the expression level of at least one gene of Table 1 in a biological sample of a lymphoma patient; and
(b) comparing the expression level of the at least one gene in the biological sample to: (i) the expression level of the at least one gene in biological samples from lymphoma patients who are responsive to the cancer treatment, and (ii) the expression level of the at least one gene in biological samples from lymphoma patients who are not responsive to the cancer treatment,
wherein if the expression level of (a) is similar to the expression level of (i), it indicates that the first lymphoma patient is likely to be responsive to the cancer treatment; and if the expression level of (a) is similar to the expression level of (ii), it indicates that the first lymphoma patient is not likely to be responsive to the cancer treatment.

31. A method of treating a lymphoma patient, comprising:
(i) identifying a lymphoma patient who is likely to be responsive to the cancer treatment according to the method of claim 30; and
(ii) administering to the lymphoma patient the cancer treatment.

32. A method of treating a lymphoma patient, comprising:
(i) identifying a lymphoma patient who is not likely to be responsive to the cancer treatment according to the method of claim 29 or 30; and
(ii) administering to the lymphoma patient an alternative cancer treatment.

33. The method of any one of claims 30-32, wherein the cancer treatment is R-CHOP.

34. The method of claim 32, wherein the alternative cancer treatment is a BET inhibitor, or a CDK inhibitor.

35. The method of any one of claims 28-34, wherein the lymphoma is selected from the group consisting of DLBCL, indolent B cell lymphoma, follicular lymphoma, small lymphocytic lymphoma, nodal marginal zone B-cell lymphoma, lymphoplasmacytic lymphoma, anaplastic large cell lymphoma, primary cutaneous type lymphoma, mycosis fungoides, chronic lymphocytic leukemia, and mantle cell Lymphoma.

36. The method of claim 35, wherein the lymphoma is DLBCL.

37. The method of claim 35, wherein the lymphoma is DLBCL, indolent B cell lymphoma, follicular lymphoma, nodal marginal zone B-cell lymphoma, mantle cell lymphoma, or chronic lymphocytic leukemia.

38. The method of any one of claims 29-37, wherein the expression levels of all genes of Table 1 are determined in (a) and compared in (b).

39. The method of any one of claims 1-38, wherein the biological samples are tumor biopsy samples.

40. The method of any one of claims 1-39, wherein determining the expression level of the at least one gene comprises detecting the presence or amount of at least one complex in the biological samples, wherein the presence or amount of the at least one complexe indicates the expression level of the at least one gene.

41. The method of claim 40, wherein the at least one complex is a hybridization complex.

42. The method of claim 40, wherein the at least one complex is detectably labeled.

43. The method of any one of claims 1-39, wherein determining the expression level of the at least one gene comprises detecting the presence or the amount of at least one reaction product in the biological samples, wherein the presence or amount of the at least one reaction product indicates the expression level of the at least one gene.

44. The method of claim 43, wherein the at least one reaction product is detectably labeled.

45. The method of any one of claims 1-44, wherein the reference lymphoma patient is a refractory DLBCL patient, a relapsed DLBCL patient, or a newly diagnosed DLBCL patient.

46. The method of any one of claims 1-45, wherein the lymphoma patient is a refractory DLBCL patient, a relapsed DLBCL patient, or a newly diagnosed DLBCL patient.

47. The method of any one of claims 1-46, wherein the lymphoma patient is a GCB DLBCL patient or an ABC DLBCL patient.

48. The method of any one of claims 1-47, wherein the lymphoma patient is a DHITsig+DLBCL patient or a DHITsig− DLBCL patient.
